(19) United States
(12) Patent Application Publication
(10) Pub. No.: US 2008/0003200 A1
(43) Pub. Date: Jan. 3, 2008

(54) GRP78 targeting peptides and methods employing same

(76) Inventors: Wadih Arap, Houston, TX (US); Mikhail G. Kolonin, Houston, TX (US); Renata Pasqualini, Houston, TX (US); Amado J. Zurita, Houston, TX (US)

Correspondence Address: FULBRIGHT & JAWORSKI L.L.P., 600 CONGRESS AVE., SUITE 2400, AUSTIN, TX, 78701, US

(21) Appl. No.: 11/835,340

(22) Filed: Aug. 7, 2007

Related U.S. Application Data
(62) Division of application No. 11/026,999, filed on Dec. 30, 2004, now abandoned.
(60) Provisional application No. 60/533,650, filed on Dec. 31, 2003.

(51) Int. Cl. A61K 38/16; A61K 31/16; A61K 31/165; A61K 31/335; A61K 31/35; A61K 31/445; A61K 38/20; A61K 38/43; A61P 35/00; C12N 7/00; C12N 5/00; C12N 15/00; C07K 7/06; C07K 2/00; A61K 38/48; A61K 38/21; A61K 38/19; A61K 31/505; A61K 31/56; A61K 38/00; A61K 38/04

(52) U.S. Cl. 424/85.2; 424/130.1; 424/184.1; 424/85.4; 424/85.6; 424/85.7; 424/94.1; 424/94.63; 435/235.1; 435/320.1; 435/325; 514/12; 514/14; 514/182; 514/2; 514/256; 514/274; 514/323; 514/44; 514/449; 514/456; 514/616; 514/619; 514/625; 514/626; 530/300; 530/328

(57) ABSTRACT

The compositions and methods include targeting peptides selective for tissue selective binding, particularly prostate and/or bone cancer, or adipose tissue. The methods may comprise targeting peptides that bind, for example, cell surface GRP78, IL-11Rα in blood vessels of bone, or prohibitin of adipose vascular tissue. These peptides may be used to induce targeted apoptosis in the presence or absence of at least one pro-apoptotic peptide. Antibodies against such targeting peptides, the targeting peptides, or their mimeotopes may be used for detection, diagnosis and/or staging of a condition, such as prostate cancer or metastatic prostate cancer.

This application is a divisional of U.S. patent application Ser. No. 11/026,999 filed Dec. 30, 2004, and claims priority to U.S. Provisional Patent application Ser. No. 60/533,650, filed on Dec. 31, 2003 entitled “Compositions and Methods of Use of Targeting Peptides for Diagnosis and Therapy,” all of which are incorporated herein by reference in their entirety.

The United States Government owns rights in this invention pursuant to NIH grants CA90270 and CA9081001.

### BACKGROUND OF THE INVENTION

I. Field of the Invention

The present invention concerns the fields of medical diagnostics, targeted delivery of therapeutic agents to cells and/or tissues. More specifically, the present invention relates to compositions and methods for identification and use of peptides that selectively target cancer cell receptors, such as the Interleukin 11 (IL-11) receptor alpha and/or the glucose regulated protein 78 (GRP78) receptor.

II. Background of the Invention

Therapeutic treatment of many conditions is limited by the systemic toxicity of the therapeutic agents used. For example, cancer therapeutic agents in particular exhibit a very low therapeutic index, with rapidly growing normal tissues such as skin and bone marrow affected at concentrations of agent that are not much higher than the concentrations used to kill tumor cells. Treatment of conditions such as cancer and other organ, tissue, or cell type confined disease states would be greatly facilitated by the development of compositions and methods for targeted delivery to a desired organ, tissue or cell type of a therapeutic agent.

Recently, an in vivo selection system was developed using phage display libraries to identify targeting peptides for various organs, tissues, or cell types in a mouse model system. Phage display libraries expressing transgenic peptides on the surface of bacteriophage were initially developed to map epitope binding sites of immunoglobulins (Smith and Scott, 1986, 1993). Such libraries can be generated by inserting random oligonucleotides into cDNAs encoding a phage surface protein, generating collections of phage particles displaying unique peptides in as many as 10⁹ permutations (Pasqualini and Ruoslahti, 1996, Arap et al., 1998a; 1998b).

Intravenous administration of phage display libraries to mice was followed by the recovery of phage from individual organs (Pasqualini and Ruoslahti, 1996). Phage were recovered that were capable of selective homing to the vascular beds of different mouse organs, tissues, or cell types, based on the specific targeting peptide sequences expressed on the outer surface of the phage (Pasqualini and Ruoslahti, 1996). A variety of organ and tumor-homing peptides have been identified by this method (Rajotte et al., 1998, 1999; Koivunen et al., 1999a; Burg et al., 1999; Pasqualini, 1999). Each of those targeting peptides bound to different receptors that were selectively expressed on the vasculature of the mouse target tissue (Pasqualini, 1999; Pasqualini et al., 2000; Folkman, 1995; Folkman 1997). Tumor-homing peptides bound to receptors that were upregulated in the tumor angiogenic vasculature of mice (Brooks et al., 1994; Pasqualini et al., 2000). In addition to identifying individual targeting peptides selective for an organ, tissue, or cell type (Pasqualini and Ruoslahti, 1996; Arap et al., 1998a; Koivunen et al., 1999b), this system has been used to identify endothelial cell surface markers that are expressed in mice in vivo (Rajotte and Ruoslahti, 1999).

This relative success notwithstanding, cell surface selection of phage libraries has been plagued by technical difficulties. A high number of non-binder and non-specific binder clones are recovered using previous in vivo methods, particularly with components of the reticuloendothelial system such as spleen and liver. Removal of this background phage binding by repeated washes is both labor-intensive and inefficient. Cells and potential ligands are frequently lost during the many washing steps required. Methods that have been successful with animal model systems are unsatisfactory for identifying human targeting peptides, which may differ from those obtained in mouse or other animal model systems.

Attachment of therapeutic agents to targeting peptides has resulted in the selective delivery of the agent to a desired organ, tissue, or cell type in the mouse model system. Targeted delivery of chemotherapeutic agents and proapoptotic peptides to receptors located in tumor angiogenic vasculature resulted in a marked increase in therapeutic efficacy and a decrease in systemic toxicity in tumor-bearing mouse models (Arap et al., 1998a, 1998b; Ellerby et al., 1999). A need exists for targeting peptides that are selective against conditions such as human tumors or that can distinguish between metastatic and non-metastatic human tumors.

Adenovirus type 5 (Ad5)-based vectors have been commonly used for gene transfer studies (Weitzman et al., 1997; Zhang, 1999). These techniques are well-known in the art. The problem with this technology is that it is not always targeted to the site of interest and unwanted side effects may occur. A need exists to develop novel gene therapy vectors to allow more selective delivery of gene therapy agents.

The need exists to identify receptor-ligand pairs in organs, tissues, or cell types. Previous attempts to identify targeted receptors and ligands binding to receptors have largely targeted a single ligand at a time for investigation. Such novel receptors and ligands may provide the basis for new therapies for a variety of disease states, such as cancer and/or metastatic prostate cancer.

### SUMMARY OF THE INVENTION

Embodiments of the invention include an isolated peptide that selectively binds IL-11 receptor-alpha (IL11Rα). The isolated peptide may comprise all or part of SEQ ID NO:1, SEQ ID NO:2, SEQ ID NO:3, SEQ ID NO:4, or SEQ ID NO:5. In certain aspects, the isolated peptide is therapeutic for the treatment of cancer or is operatively coupled to a therapeutic agent. In other aspects, the cancer is cancer, prostate cancer, or metastatic prostate cancer expressing IL11Rα. The isolated peptide may be covalently coupled to a therapeutic agent. Therapeutic agent include a drug, a chemotherapeutic agent, a radioisotope, a pro-apoptosis agent, an anti-angiogenic agent, a hormone, a cytokine, a cytotoxic agent, a cytocidal agent, a cytostatic agent, a peptide, a protein, an antibiotic, an antibody, a Fab fragment of an antibody, a hormone antagonist, a nucleic acid or an antigen. An anti-angiogenic agent may include thrombospondin, angiostatin5, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, Docetaxel, polyamines, a proteasome inhibitor, a kinase inhibitor, a signaling peptide, accutin, cidofovir, vincristine, bleomycin, AGM-1470, platelet factor 4 and minocycline. In still a further aspect, a pro-apoptosis agent may include etoposide, ceramide sphingomyelin, Bax, Bid, Bik, Bad, caspase-3, caspase-8, caspase-9, fas, fas ligand, fadd, fap-1, tradd, faf, rip, reaper, apoptin, interleukin-2 converting enzyme or annexin V. In yet still a further aspect, a cytokine may include interleukin 1 (IL-1), IL-2, IL-5, IL-10, IL-12, IL-18, interferon-γ (IF-γ), IF-α, IF-β, tumor necrosis factor-α (TNF-α), or GM-CSF (granulocyte macrophage colony stimulating factor).

The isolated peptide of the invention can be attached to a molecular complex. A complex may include a virus, a bacteriophage, a bacterium, a liposome, a microparticle, a magnetic bead, a yeast cell, a mammalian cell or a cell. In particular embodiments, the complex is a virus or a bacteriophage. A virus includes, but is not limited to adenovirus, retrovirus, or adeno-associated virus (AAV). A virus may be a gene therapy vector containing a therapeutic nucliec acid or a gene therapy. In certain aspects the peptide is attached to a eukaryotic expression vector, preferably a gene therapy vector. Compositions comprising the isolated peptide will typically be comprised in a pharmaceutically acceptable composition.

In further embodiments the invention includes a nucleic acid that encodes a protein or peptide comprising all or part of SEQ ID NO:1, SEQ ID NO:2, SEQ ID NO:3, SEQ ID NO:4, or SEQ ID NO:5. In certain aspects, the nucleic acid is operably linked to a heterologous promoter.

In still further embodiments, the invention includes methods of treating cancer comprising administering a peptide that selectively binds a IL-11Rα to a subject. In other aspects the peptide(s) inhibit growth of a cancer cell. In certain embodiments the cancer is prostate cancer. In still further embodiments the prostate cancer is metastatic prostate cancer. In certain aspects the subject is a mammal, preferably a human. The peptide may be administered in a pharmaceutically acceptable carrier. The methods of the invention may include administering a second therapeutic agent to the subject.

In still further embodiments of the invention include methods for imaging cells expressing IL-11Rα comprising exposing cells to an isolated peptide that selectively binds IL-11Rα, wherein the peptide is coupled to a second agent. The second agent may include a radioisotope or an imaging agent. Furthermore, the cells to be imaged may be prostate cells, preferably metastatic prostate cells.

Embodiments of the invention include an isolated peptide that selectively binds IL-11Rα, identified by a process comprising: a) contacting a cell or tissue expressing IL-11Rα with a plurality of phage, wherein each phage comprises heterologous peptide sequences incorporated into a fiber protein, b) removing the phage that do not bind to the cell or tissue expressing IL-11Rα, and c) isolating the phage that bind the cell or tissue expressing IL-11Rα. In certain aspects the method is repeated at least twice. The peptide may further comprise isolating and sequencing the isolated phage nucleic acid. In other aspects the IL-11Rα expression is endogenous to the cell or tissue utilized or exogenous to the cell or tissue utilized, e.g., expressed from an expression construct.

As used herein in the specification, “a” or “an” may mean one or more. As used herein in the claim(s), in conjunction with the word “comprising,” the words “a” or “an” may mean one or more than one. As used herein “another” may mean at least a second or more of an item.

A “targeting peptide” is a peptide comprising a contiguous sequence of amino acids, which is characterized by selective localization to an organ, tissue, or cell type. Selective localization may be determined, for example, by methods disclosed below, wherein the putative targeting peptide sequence is incorporated into a protein that is displayed on the outer surface of a phage. Administration to a subject of a library of such phage that have been genetically engineered to express a multitude of such targeting peptides of different amino acid sequence is followed by collection of one or more organs, tissues, or cell types from the subject and identification of phage found in that organ, tissue, or cell type. A phage expressing a targeting peptide sequence is considered to be selectively localized to a tissue or organ if it exhibits greater binding in that tissue or organ compared to a control tissue or organ. Preferably, selective localization of a targeting peptide should result in a two-fold or higher enrichment of the phage in the target organ, tissue, or cell type, compared to a control organ, tissue, or cell type. Selective localization resulting in at least a three-fold, four-fold, five-fold, six-fold, seven-fold, eight-fold, nine-fold, ten-fold or higher enrichment in the target organ compared to a control organ, tissue or cell type is more preferred. Alternatively, a phage expressing a targeting peptide sequence that exhibits selective localization preferably shows an increased enrichment in the target organ compared to a control organ when phage recovered from the target organ are reinjected into a second host for another round of screening. Further enrichment may be exhibited following a third round of screening. Another alternative means to determine selective localization is that phage expressing the putative target peptide preferably exhibit a two-fold, more preferably a three-fold or higher enrichment in the target organ or tissue compared to control phage that express a non-specific peptide or that have not been genetically engineered to express any putative target peptides. Another means to determine selective localization is that localization to the target organ or tissue of phage expressing the target peptide is at least partially blocked by the co-administration of a synthetic peptide containing the target peptide sequence. “Targeting peptide” and “homing peptide” are used synonymously herein.

A “phage display library” means a collection of phage that have been genetically engineered to express a set of putative targeting peptides on their outer surface. In preferred embodiments, DNA sequences encoding the putative targeting peptides are inserted in frame into a gene encoding a phage capsule protein. In other preferred embodiments, the putative targeting peptide sequences are in part random mixtures of all twenty amino acids and in part non-random. In certain preferred embodiments, the putative targeting peptides of the phage display library exhibit one or more cysteine residues at fixed locations within the targeting peptide sequence. Cysteines may be used, for example, to create a cyclic peptide.

A “macromolecular complex” refers to a collection of molecules that may be random, ordered or partially ordered in their arrangement. The term encompasses biological organisms such as bacteriophage, viruses, bacteria, unicellular pathogenic organisms, multicellular pathogenic organisms and prokaryotic or eukaryotic cells. The term also encompasses non-living assemblages of molecules, such as liposomes, microcapsules, microparticles, magnetic beads and microdevices. The only requirement is that the complex contains more than one molecule. The molecules may be identical, or may differ from each other.

A “receptor” for a targeting peptide includes but is not limited to any molecule or macromolecular complex that binds to a targeting peptide. Non-limiting examples of receptors include peptides, proteins, glycoproteins, lipoproteins, epitopes, antibodies, lipids, carbohydrates, multi-molecular structures, a specific conformation of one or more molecules and a morphoanatomic entity. In preferred embodiments, a “receptor” is a naturally occurring molecule or complex of molecules that is present on the cell or the lumenal surface of cells forming blood vessels within or supplying nutrients to a target organ, tissue, or cell type.

A “subject” refers generally to a mammal. In certain preferred embodiments, the subject is a mouse or rabbit. In even more preferred embodiments, the subject is a human.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1I illustrate an example of IL-11Rα expression in normal prostate and primary and metastatic prostate cancer. FIG. 1A shows normal glands from the peripheral zone showing predominant staining in the basal cell compartment and area of transitional metaplasia (arrow), and no staining in the luminal cell layers. FIG. 1B shows strong (3+) positive staining in high-grade primary androgen-dependent prostatic adenocarcinoma. FIG. 1C shows homogeneous (3+) expression in prostate cancer metastatic to bone. FIG. 1D is a negative control (normal Ig). FIG. 1E is a positive staining in small blood vessels around malignant tumor tissue in bone matrix, confirmed by CD31 immunostaining on serial tissue sections (see inset for a representative section). FIGS. 1F and 1G are IL-11-mimic phage overlays. FIG. 1F is a high-grade, androgen-independent primary tumor showing strong (3+) and homogeneous staining in malignant epithelium and associated vessels (arrows). FIG. 1G is a strong homogeneous expression in prostate cancer metastatic to bone. FIGS. 1H and 1I are IL-11-mimic phage-staining inhibition. Phage localization to primary prostate cancer glands (FIG. 1H) was abolished (serial tissue sections) by co-incubation with soluble CGRRAGGSC (SEQ ID NO:1)—GG-_{D}(KLAKLAK)₂ (SEQ ID NO:11) peptide (FIG. 1I). Bar, 50 μm in all panels.

FIGS. 2A-2D represent an example of a control and experimental peptide, CGRRAGGSC (SEQ ID NO:1)-GG-_{D}(KLAKLAK)₂ (SEQ ID NO:11) that binds specifically to IL-11Rα and induces apoptosis in IL-11Rα-positive prostate cancer cell lines.

FIGS. 3A-3F These FIGs. represent an example of a phage carrying peptide that (IL-11-mimic phage) internalizes and induces programmed cell death (CGRRAGGSC (SEQ ID NO:1)-GG-_{D}(KLAKLAK)₂ (SEQ ID NO:11) synthetic peptide). FIG. 3A shows a IL-11-mimic phage internalization on LNCaP cells. Note distribution in cell projections and around the nucleus (inset). FIG. 3B shows an insertless fd phage was used as negative control for internalization (phase-contrast in inset). FIGS. 3C, 3D, 3E and 3F, induction of programmed cell death with CGRRAGGSC (SEQ ID NO:1)-GG-_{D}(KLAKLAK)₂ (SEQ ID NO:11) synthetic peptide. LNCaP (FIGS. 3C and 3D) or MDA-PCa-2b (FIGS. 3E and 3F) cells were incubated with 50 μM CGRRAGGSC (SEQ ID NO:1)-GG-_{D}(KLAKLAK)₂ (SEQ ID NO:11) (FIGS. 3C and 3E) or an equimolar mixture of unconjugated CGRRAGGSC (SEQ ID NO:1)- and _{D}(KLAKLAK)₂ (SEQ ID NO:11) (FIGS. 3D and 3F.)

FIGS. 4A-4E. FIG. 4A is a schematic representation of phage displaying peptides binding to a target on the cell surface. This figure represents any ligand-receptor pair. FIGS. 4B, 4C, 4D and 4E represent an example of the binding and specificity of WIFPWIQL (SEQ ID NO:6)-phage (FIG. 4A) and of WDLAWMFRLPVG (SEQ ID NO:7)-phage (FIG. 4B) to recombinant GRP78 in microtiter wells. FIGS. 4C and 4D represent a dose-dependent inhibition of WIFPWIQL (SEQ ID NO:6)-phage (FIG. 4C) and WDLAWMFRLPVG (SEQ ID NO:7)-phage (FIG. 4D).

FIGS. 5A and 5B represents an example of the binding of filamentous phage clones displaying WIFPWIQL (SEQ ID NO:6) (FIG. 5A) and WDLAWMFRLPVG (SEQ ID NO:7) (FIG. 5B) to intact DU145 human prostate cancer cells by using an aqueous-organic phase separation.

FIG. 6 represents an example of the ability of GRP78-binding phage clones to home to tumors in vivo, the selected phage or control phage were intravenously injected into nude mice bearing DU145-derived xenografts.

FIGS. 7A and 7B represents the binding of the GRP78-binding phage to human prostate cancer bone metastases by phage overlay assays was tested, an anti-GRP78 antibody was added to a slide (FIG. 7A), and a control antibody was added to a slide (FIG. 7B).

FIG. 8 represents an example testing whether the GRP78-binding phage could inhibit the anti-GRP78 antibody staining, both GRP78-binding phage were incubated prior to the antibody and a control phage was also used.

FIG. 9 represents a test of the efficacy of the WIFPWIQL (SEQ ID NO:6)-GG-_{D}(KLAKLAK)₂ (SEQ ID NO:11) and WDLAWMFRLPVG (SEQ ID NO:7)-GG-_{D}(KLAKLAK)₂ (SEQ ID NO:11) peptides in different GRP78-expressing prostate cancer cell lines, as verified by Annexin-V staining.

FIG. 10 represents peptides tested to see whether they have anti-cancer activity in vivo, using human prostate cancer xenografts. Individual tumor volumes before and after treatment are represented.

FIGS. 11A-11F represent an example of in vivo fat homing of the CKGGRAKDC motif in genetically obese mice. FIGS. 11A, 11C, 11E, and 11F represent Anti-phage immunohistochemistry; or FIGS. 11B and 11D represent immunostaining control (no primary anti-phage antibody) in paraffin sections of formalin-fixed tissues from ob/ob mice intravenously injected 12 hr prior to tissue processing with CKGGRAKDC (SEQ ID NO:4)-phage (FIGS. 11A, 11 B and 11E), or control insertless phage (FIGS. 11C, 11D, and 11F). Homing of the CKGGRAKDC (SEQ ID NO:4) peptide to fat blood vessels (arrows) is indicated. Hematoxylin counter-staining is grey. Scale bar, 50 μm.

FIGS. 12A-12F represent In vivo fat homing of the CKGGRAKDC (SEQ ID NO:4) motif in wild-type mice. FIGS. 12A, 12C, 12D, 12E and 12F green immunofluorescence; or FIG. 12B, red immunofluorescence in formalin-fixed paraffin sections of white fat (FIGS. 12A, 12B, and 12C), brown fat (FIGS. 12D and 12F), or liver (FIG. 12E) from C57BL/6 mice intravenously injected 5 min prior to tissue processing with CKGGRAKDC (SEQ ID NO:4)-fitc peptide and lectin-rhodamine (FIGS. 12A, 12B, 12D, and 12E), or control scrambled CGDKAKGRC (SEQ ID NO:10)-fitc peptide and lectin-rhodamine (FIGS. 12C and 12F). Homing of the CKGGRAKDC (SEQ ID NO:4) peptide to white fat vasculature (arrows) and endothelium marked with lectin-rhodamine (arrows) is indicated. Only green autofluorescence is detected for CGDKAKGRC (SEQ ID NO:10) in control organs or for the control peptide in all organs. Scale bar, 50 μm.

FIGS. 13A-13G illustrate the physiological effects of treatment with CKGGRAKDC (SEQ ID NO:4)-GG-_{D}(KLAKLAK)₂ (SEQ ID NO:11). Cohorts (n=2×8) of diet-induced obese C57BL/6 mice were subcutaneously injected with 150 μg CKGGRAKDC-GG-_{D}(KLAKLAK)₂ (▪ treated) or an equimolar mixture of CKGGRAKDC (SEQ ID NO:4) and _{D}(KLAKLAK)₂ (SEQ ID NO:11) (□ control) peptides daily. (FIG. 13A) Weight loss in response to treatment (average from two independent experiments). (FIG. 13B) The appearance of representative treated and control mice and their epididymal fat depots at the end of the treatment course. (FIG. 13C) Serum concentration of non-essential fatty acids (NEFA), glycerol, triacylglycerol (TAG), and cholesterol at the end of the treatment course. (FIG. 13D) Paraffin sections of livers and soleus skeletal muscle from mice shown in (FIG. 13B) stained with hematoxylin/eosin showing resorption of fat in livers of mice treated for 4 weeks (scale bar, 50 μm). (FIG. 13E) Total lipid content in liver and soleus+gastrocnemius skeletal muscle of treated and control mice at the end of the treatment course. (FIG. 13F) Serum leptin level in treated and control mice after 4 weeks of treatment. (FIG. 13F) Mean daily food consumption per kg of body weight by treated and control mice averaged for the first and second bi-weekly treatment intervals. Error bars are s.d. for 16 mice (FIG. 13A) or s.e.m. for 8 mice (FIGS. 13C, 13D, 13E, 13F and 13G).

FIGS. 14A-14D represents the destruction of fat blood vessels as a result of targeted apoptosis. TUNEL immunohistochemistry (FIGS. 14A, 14B, and 14D), or secondary antibody only negative TUNEL staining control (FIG. 14C) of white fat (FIGS. 14A, 14B, and 14C) or a control organ (liver, FIG. 14D) of mice treated with CKGGRAKDC (SEQ ID NO:4)-GG-_{D}(KLAKLAK)₂ (SEQ ID NO:11) (a, c, d) or CARAC (SEQ ID NO:9)-GG-_{D}(KLAKLAK)₂ (SEQ ID NO:11) control (FIG. 14B) for 4 weeks. Apoptosis (HRP staining; arrows) induced by CKGGRAKDC (SEQ ID NO:4)-GG-_{D}(KLAKLAK)₂ (SEQ ID NO:11) treatment is indicated. Hematoxylin counter-staining is blue. Scale bar, 25 μm.

Deleted

FIGS. 15A-15H illustrate that Prohibitin is the target of CKGGRAKDC (SEQ ID NO:4) in white fat. (FIG. 15A) Sepharose4B (Column) unloaded (Mock) or loaded with CKGGRAKDC (SEQ ID NO:4)-gst (Targ.) or a control white fat-homing peptide, CVMGSVTGC (SEQ ID NO:12)-gst (Ctrl.), was incubated with in vivo-biotinylated membrane extract from mouse white fat. Bound proteins were eluted with CKKRAKDC (SEQ ID NO:4)-fitc (Targ.) or CVMGSVTGC (SEQ ID NO:12)-fitc control peptide (Ctrl.), resolved by 4-20% SDS-PAGE and detected by immunoblotting with streptavidin-conjugated antibodies; (FIGS. 15C and 15E) EAH Sepharose loaded with CKGGRAKDC (SEQ ID NO:4)-GG-_{D}(KLAKLAK)₂ (SEQ ID NO:11) (Targ.) or CARAC (SEQ ID NO:9)-GG-_{D}(KLAKLAK)₂ (SEQ ID NO:11) control peptide (Ctrl.) was incubated with membrane extract from mouse white fat. Bound proteins were eluted with low pH and resolved by 4-20% (FIG. 15B) or by 12% (c) SDS-PAGE and stained with Coomassie blue (FIG. 15B); or immunoblotted with anti-prohibitin antibody (FIG. 15C). M=molecular weight marker. Arrowheads: migration of the 35 kDa prohibitin. (FIG. 15D) Recombinant gst-fused prohibitin, unrelated gst fusion (control-gst), or BSA immobilized on a microtiter plate were incubated with the CKGGRAKDC (SEQ ID NO:4)-displaying phage with □ or without ▪ blocking with anti-prohibitin antibody, or the control insertless phage (fd-tet) with □ or without □ blocking with anti-prohibitin antibody. Binding (mean ±s.e.m.; n=3 experiments) was evaluated by quantification of bound phage transforming units (TU). (FIGS. 15E, 15F, 15G and 15H) Immunohistochemistry (polyclonal anti-prohibitin antibody) on formalin-fixed paraffin sections of mouse adipose tissue (FIG. 15E), and pancreas (FIG. 15F), or human white adipose tissue (FIG. 15G) and dedifferentiated liposarcoma (FIG. 15H) demonstrates selective prohibitin expression in white adipose blood vessels (arrows). Asterisk (*): prohibitin in mitochondria of bordering mouse brown fat. Hematoxylin counter-staining in (e-h) is grey. Scale bar, 25 μm.

### DESCRIPTION OF ILLUSTRATIVE EMBODIMENTS

The present invention provides additional compositions and methods for cell and/or tissue targeting, as well as compositions and methods for the use of targeted peptides that bind particular proteins or circulating antibodies. In certain embodiments, targeting peptides are selective and/or specific for human cancer tissues, such as metastatic prostate cancer. In other embodiments, targeting peptides are selective for adipose tissue and may be used to treat the condition of obesity.

Certain aspects of the invention are directed to isolated peptides of 100 amino acids or less in size, comprising at least 3 contiguous amino acids of a targeting peptide sequence that selective binds a cancer cell, a prostate cancer cell, a metastatic cancer cell, a metastatic prostate cancer cell, or adipose tissue/cells, preferably expressing, aberrantly expressing or over expressing an IL11Rα, GRP78 polypeptide or other tissue or cell selective receptor(s). Targeting peptides include but are not limited to those of SEQ ID NO:1-10. An isolated peptide may be 50 amino acids or less, more preferably 30 amino acids or less, more preferably 20 amino acids or less, more preferably 10 amino acids or less, or even more preferably 5 amino acids or less in size. In other aspects of the invention, an isolated peptide may comprise at least 4, 5, 6, 7, 8 or 9 contiguous amino acids of a targeting peptide sequence, which includes, but is not limited to the amino acids of SEQ ID NO:1-10.

In still a further aspect, an isolated peptide may be attached to a second molecule. In preferred embodiments, the attachment is a covalent attachment. The molecule may be therapeutic agent including, but not limited to a drug, a chemotherapeutic agent, a radioisotope, a pro-apoptosis agent, an anti-angiogenic agent, a hormone, a cytokine, a growth factor, a cytotoxic agent, a peptide, a protein, an antibiotic, an antibody, a Fab fragment of an antibody, a survival factor, an anti-apoptotic factor, a hormone antagonist, an imaging agent, a nucleic acid or an antigen. Those molecules are representative only and virtually any molecule may be attached to a targeting peptide and/or administered to a subject. In preferred embodiments, the pro-aptoptosis agent is gramicidin, magainin, mellitin, defensin, cecropin, (KLAKLAK)₂ (SEQ ID NO:11). In other preferred embodiments, the anti-angiogenic agent is angiostatin5, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, docetaxel, polyamines, a proteasome inhibitor, a kinase inhibitor, a signaling inhibitor (SU5416, SU6668, Sugen, South San Francisco, Calif.), accutin, cidofovir, vincristine, bleomycin, AGM-1470, platelet factor 4 or minocycline. In still further embodiments, the cytokine is interleukin 1 (IL-1), IL-2, IL-5, IL-10, IL-11, IL-12, IL-18, interferon-γ (IF-γ), IF-α, IF-β, tumor necrosis factor-α (TNF-α), or GM-CSF (granulocyte macrophage colony stimulating factor). Such examples are representative only and are not intended to exclude other pro-apoptosis agents, anti-angiogenic agents or cytokines known in the art.

In certain aspects, targeting peptides attached to one or more therapeutic agents may be administered to a subject, such as a human subject. Such administration may be of use for the treatment of various disease states. In certain embodiments, cancer-targeting peptides attached to a cytocidal, pro-apoptotic, anti-angiogenic or other therapeutic agent may be of use in methods to treat human cancer. In certain embodiments, adipose-targeting peptides attached to a cytocidal, pro-apoptotic, anti-angiogenic or other therapeutic agent may be of use in methods to treat obesity, induce weight loss and/or to treat highly active antiretroviral therapy (HAART) associated lipodystrophy syndrome.

In other aspects of the invention, an isolated peptide may be attached to a macromolecular complex. In preferred embodiments, the macromolecular complex is a virus, a bacteriophage, a bacterium, a liposome, a microparticle, a magnetic bead, a yeast cell, a mammalian cell, a cell, or a microdevice. These are representative examples only and macromolecular complexes within the scope of the present invention may include virtually any complex that may be attached to a targeting peptide and administered to a subject. In other preferred embodiments, the isolated peptide may be attached to a eukaryotic expression vector, more preferably a gene therapy vector.

Various aspects of the invention concern targeted gene therapy vectors, comprising targeting peptides, which may be encoded by the nucleic acid encoding a surface protein of a vector, expressed on the surface of a gene therapy vector. In particular embodiments, a targeted gene therapy vector is a chimeric phage-based vector containing elements from adeno-associated virus (AAV), the modified vector being referred to as an adeno-associated phage (AAP) vector.

In another embodiment, the targeting peptides may be attached to a solid support, preferably magnetic beads, Sepharose beads, agarose beads, a nitrocellulose membrane, a nylon membrane, a column chromatography matrix, a high performance liquid chromatography (HPLC) matrix or a fast performance liquid chromatography (FPLC) matrix. Such immobilized peptides may be used, for example, for affinity purification of various components, such as receptors or antibodies.

Additional aspects of the present invention concern fusion proteins comprising at least 3, 4, 5, 6, 7 or more contiguous amino acids of a targeting peptide, including sequences selected from any of SEQ ID NO:1-10. In some embodiments, larger contiguous sequences, up to a full-length sequence selected from any of SEQ ID NO:1-12 and combinations thereof.

Certain other embodiments concern compositions comprising isolated targeting peptides or fusion proteins comprising a targeting peptide in a pharmaceutically acceptable carrier.

Certain methods concern the targeted delivery to a desired organ, tissue or cell type, such as prostate cancer, by attaching the targeting peptide to a molecule, macromolecular complex or gene therapy vector, and providing the peptide attached to the molecule, complex or vector to a subject. Preferably, the targeting peptide is selected to include at least 3 or more contiguous amino acids from any of SEQ ID NO:1-12. In other preferred embodiments, the molecule attached to the targeting peptide is a chemotherapeutic agent, an antigen or an imaging agent. In various embodiments, methods of targeted delivery may utilize antibodies against particular peptide sequences, such as SEQ ID NO:1-12. Such antibodies may be attached to a molecule, macromolecular complex or gene therapy vector and administered to a subject. The skilled artisan will realize that the targeting moiety is not limited to antibodies, but may comprise any molecule or complex that binds to a receptor located in a target tissue, including but not limited to antibodies, genetically engineered antibodies, antibody fragments, single-chain antibodies, humanized antibodies, chimeric antibodies, binding proteins and native ligands or homologs thereof. In preferred embodiments of the invention, the targeted receptor is GRP78 or IL-11Rα. In other preferred embodiments, the targeted tissue is adipose tissue and more particular the targeted tissue is the vasculature components of adipose tissue.

In certain embodiments, targeting peptides and/or antibodies disclosed herein may be of use for the detection, diagnosis and/or prognosis of human cancer, such as prostate cancer. In preferred embodiments, the targeting peptides may be used to differentially diagnose metastatic and non-metastatic prostate cancer. In other embodiments, a targeting peptide may be used to target adipose tissue of a patient suffering from obesity or other condition.

Embodiments of the present invention concern isolated nucleic acids of 300 nucleotides or less in size, encoding a targeting peptide. In preferred embodiments, the isolated nucleic acid is 250, 225, 200, 175, 150, 125, 100, 75, 50, 40, 30, 20 or even 10 nucleotides or less in size. In other preferred embodiments, the isolated nucleic acid is incorporated into a eukaryotic or a prokaryotic expression vector. In even more preferred embodiments, the vector is a plasmid, a cosmid, a yeast artificial chromosome (YAC), a bacterial artificial chromosome (BAC), a virus or a bacteriophage. In other preferred embodiments, the isolated nucleic acid is operatively linked to a leader sequence that localizes the expressed peptide to the extracellular surface of a host cell.

Additional embodiments of the present invention concern methods of treating a condition, such as cancer, or obesity comprising selecting a targeting peptide and/or antibody against a selected peptide that targets cells associated with the disease state, attaching one or more molecules effective to treat the condition to the peptide, and administering the peptide to a subject with the disease state. Preferably, the peptide includes at least three contiguous amino acids selected from any of selected from any of SEQ ID NO:1-12.

In certain embodiments, the methods concern Biopanning and Rapid Analysis of Selective Interactive Ligands (BRASIL), a method for phage display that results in decreased background of non-specific phage binding, while retaining selective binding of phage to cell receptors.

In other embodiments, phage that bind to a target organ, tissue, or cell type, for example to prostate cancer cells or tissue, may be pre-screened or post-screened against a subject lacking that organ, tissue, or cell type, such as a female subject with regard to prostate selectivity. Phage that bind to a control subject are removed from the library prior to screening in subjects possessing the organ, tissue, or cell type.

In preferred embodiments, targeting phage may be recovered from specific cell types or sub-types present in an organ or tissue after selection of the cell type by PALM (Positioning and Ablation with Laser Microbeams). PALM allows specific cell types to be selected from, for example, a thin section of an organ or tissue. Phage may be recovered from the selected sample.

In another embodiment, a phage display library displaying the antigen binding portions of antibodies from a subject is prepared, the library is screened against one or more antigens. Phage that bind to the antibodies are collected. In more preferred embodiments, the antigen is a targeting peptide.

In certain embodiments, the methods and compositions may be used to identify one or more receptors and/or components for a targeting peptide. In alternative embodiments, the compositions and methods may be used to identify naturally occurring ligands for known or newly identified receptors. In preferred embodiments, the receptor may be selectively or specifically expressed in prostate cancer. In some embodiments, expression of the receptor may be up regulated in prostate cancer compared to normal prostate, and/or in metastatic compared to non-metastatic prostate cancer. Methods of diagnosis and/or prognosis of cancer, such as prostate cancer, may comprise detection and/or quantification of such disease-state selective or specific receptors in tissue samples. In some embodiments, detection and/or quantification may take place in situ within an intact subject, for example by attaching an imaging agent to an antibody or equivalent molecule that binds to the receptor.

In some embodiments, the methods may comprise contacting a targeting peptide to an organ, tissue, or cell containing a receptor of interest, allowing the peptide to bind to the component, and identifying the component by its binding to the peptide. In preferred embodiments, the targeting peptide contains at least three contiguous amino acids selected from any of selected from any of SEQ ID NO:1-12. In other preferred embodiments, the targeting peptide may comprise a portion of an antibody against the receptor. In more preferred embodiments, the antibody or antibody portion may bind to SEQ ID NO:1-12.

In alternative embodiments, the targeting peptide may contain a random amino acid sequence. The skilled artisan will realize that the contacting step can utilize intact organs, tissues, or cells, or may alternatively utilize homogenates or detergent extracts of the organs, tissues or cells. In certain embodiments, the cells to be contacted may be genetically engineered to express a suspected receptor for the targeting peptide. In a preferred embodiment, the targeting peptide is modified with a reactive moiety that allows its covalent attachment to the site of interest. In a more preferred embodiment, the reactive moiety is a photoreactive group that becomes covalently attached to the receptor when activated by light. In another preferred embodiment, the peptide is attached to a solid support and the component is purified by affinity chromatography. In other preferred embodiments, the solid support comprises magnetic beads, sepharose beads, agarose beads, a nitrocellulose membrane, a nylon membrane, a column chromatography matrix, a high performance liquid chromatography (HPLC) matrix or a fast performance liquid chromatography (FPLC) matrix.

In certain embodiments, the targeting peptide may inhibit the activity of a component such as a receptor upon binding to the component. The skilled artisan will realize that component activity can be assayed by a variety of methods known in the art, including but not limited to catalytic activity and binding activity. In other embodiments, binding of a targeting peptide to for example a receptor may inhibit a transport activity of the receptor.

In alternative embodiments, one or more ligands for a receptor of interest may be identified by the disclosed methods and compositions. One or more targeting peptides that mimic part or all of a naturally occurring ligand may be identified by phage display and biopanning in vivo or in vitro. A naturally occurring ligand may be identified by homology with a single targeting peptide that binds to the receptor, or a consensus motif of sequences that bind to the receptor. In other alternative embodiments, an antibody may be prepared against one or more targeting peptides that bind to a receptor of interest. Such antibodies may be used for identification or immunoaffinity purification of the native ligand.

In certain embodiments, the targeting peptides of the present invention are of use for the selective delivery of therapeutic agents, including but not limited to gene therapy vectors and fusion proteins, to specific organs, tissues, or cell types. The skilled artisan will realize that the scope of the claimed methods of use include any condition that can be treated by targeted delivery of a therapeutic agent to a desired organ, tissue, or cell type. Although such conditions include those where the affected cells are confined to a specific organ, tissue or cell type, other disease conditions may be treated by an organ, tissue, or cell type-targeting approach. In particular embodiments, the organ, tissue, or cell type may comprise prostate cancer tissue.

Certain embodiments concern methods of obtaining antibodies against an antigen. In preferred embodiments, the antigen comprises one or more targeting peptides. The targeting peptides may be prepared and immobilized on a solid support, serum-containing antibodies is added and antibodies that bind to the targeting peptides may be collected.

I. Targeting of Cancer Cells

In some embodiments, the invention concerns particular targeting peptides selective or specific for prostate cancer or other cancers over expressing certain receptor polypeptides, including but not limited to SEQ ID NO:6 and SEQ ID NO:7. Other embodiments concern such targeting peptides attached to therapeutic agents. In other embodiments, cancer-targeting peptides may be used to selectively or specifically deliver therapeutic agents to target tissues, such as prostate cancer and/or metastatic prostate cancer. In certain embodiments, the subject methods concern the preparation and identification of targeting peptides selective or specific for a given target cell, tissue or organ, such as prostate cancer.

A. IL-11 Receptor-Alpha (IL11Rα)

Circulating phage displaying 47,160 different peptide motifs localize to different organs in a non-random fashion, and allow the identification of several candidate human proteins mimicked by selected motifs. One example is IL-11. IL11 belongs to the gp130 family of cytokines, which includes interleukin-6 (IL6), leukemia inhibitory factor (LIF), and oncostatin M (OSM), among others. The IL11Rα chain is responsible for the IL11-binding specificity, and this complex triggers the activation of the ubiquitously expressed glycoprotein 130 (gp 130), which then initiates several signal transduction cascades. So far IL11Rα has been characterized on human solid tumours such as breast, colon, and ovary. However, the functional significance of its expression is not well understood. IL11Rα expression has been reported as increased in primary prostate carcinoma compared to non-malignant prostate tissue, in a previous report by Campbell et al. (2001a) on a limited number of samples. As an initial step to targeting up-regulated IL11Rα in the context of human prostate cancer, a study to expand previous conclusions was done by performing an extensive immunohistochemical analysis of the IL11Rα expression on both primary and metastatic prostate cancer specimens.

IL-11 initiates signaling via binding to the IL-11Rα chain. The complex of IL-11 and IL-11Rα then binds to and induces clustering of gp130, leading to the activation of associated Janus kinases (JAKs) and translocation to the nucleus of the signal transducers and activators of transcription (STAT) proteins 3 and 1 (Lutticken et al., 1994; Campbell et al., 2001a). STAT3 has been reported as constitutively activated in prostate cancer (Ni et al., 2002). IL-11Rα expression was reported to increase in primary prostatic carcinoma compared to non-malignant prostate tissue (Campbell et al., 2001a). No previous reports have characterized IL-11Rα expression in metastatic cancer.

Other signaling systems that may be activated by IL-11Rα include MAP kinase, and the ribosomal S6 protein kinase pp 90rsk, SRC-family tyrosine kinases including p60src and p62yes, and phosphatidylinositol-3 kinase. IL-11Rα has been characterized on human solid tumors such as breast, colon, ovary, and melanoma (Douglas et al., 1997; Gupta et al., 1997; Paglia et al., 1995; Campbell et al, 2001b), although its functional role and prognostic significance were unknown.

Exemplary IL-11Rα targeting peptides include CGRRAGGSC (SEQ ID NO:1), CRGSGAGRC (SEQ ID NO:2), CSGGGRARC (SEQ ID NO:3), CKGGRAKDC (SEQ ID NO:4), and CGSPGWVRC (SEQ ID NO:5).

No differences were observed in IL-11Rα expression between normal glands in the different prostatic areas (Table 1). Some background, distinct to a frequent stromal staining, was observed in the epithelium of seminal vesicles and ejaculatory ducts. Expression in PIN and AD samples examined was significantly higher than in their benign counterparts from the same areas (p<0.0001 in both cases, Wilcoxon signed rank test), but no differences were observed between PIN and AD (p=0.5, signed rank test). Among primary AD specimens, IL-11Rα immunoreactivity was increased in cancers from the peripheral vs. transition zone (p=0.0003), in Gleason≧7 (4+3) vs. Gleason≦7 (3+4) (p=0.004), and, more marginally, in pT_{3b}-pT_{any}pN₁ tumours vs. pT₂-pT₃ₐ (p=0.046) (Table 1).

Primary AI specimens showed a more homogeneous pattern of staining, with more than 80% cells displaying moderate/strong intensity in 80% of the samples. However, no significant increase in expression was observed in AI vs. AD cases matched by Gleason score (p=0.15, rank-sum test), likely because of the small number of samples. Expression in 6 regional (4 AD and 2 AI) and 6 distant lymph node metastases (6 AI) was also intense in a high percentage of tumour cells. Cancer cells displayed a homogeneous moderate to strong intensity of staining in 5 out of 6 specimens from bone metastases (all AI). Both osteoblasts and osteoclasts stained moderately, and were used as internal positive controls. Interestingly, blood vessels in bone and lymph node metastases and in primary cases with previous treatment, showed an occasionally striking IL11Rα immunoreactivity that was confirmed by CD31 staining on consecutive slides, as opposed to a more random pattern in the other benign and malignant tissues analysed.

TABLE 1
Clinical and histopathological characteristics
and IL11Rα expression

| | | | |
|---|---|---|---|
| | Number | | |
| Specimen | of cases | Median score (range)* | P |

Normal prostate

| | | | | |
|---|---|---|---|---|
| Peripheral zone | 62 | 1+ | (1-2) | NS§ |
| Transition zone | 51 | 1+ | (1-2) | |
| Central zone | 40 | 1+ | (1-2) | |

| | | | |
|---|---|---|---|
| Seminal vesicle/ | 43/3 | 2+ (2-3)/2+ (2) | . . . |
| Ejaculatory Duct | | | |

Benign pathologic conditions

| | | | | |
|---|---|---|---|---|
| Benign prostatic | 15 | 1+ | (1-2) | . . . |
| hyperplasia | | | | |
| Stromal nodule | 2 | 1+ | (1-2) | . . . |
| Atrophy | 10 | 2+ | (1-2) | . . . |
| Transitional metaplasia | 18 | 2+ | (1-2) | . . . |
| Prostatic intraepithelial | 23 | 2+ | (1-3) | . . . |
| neoplasia (PIN) | | | | |

Primary prostate cancer

| | | | |
|---|---|---|---|
| Androgen-dependent | 71 | 2 + (1-3)/180 (50-290) | . . . |

Zonal origin

| | | | | |
|---|---|---|---|---|
| Peripheral zone | 55 | 190 | (50-290) | 0.0003∥ |
| Transition zone | 16 | 135 | (50-250) | |

Gleason score†

| | | | | |
|---|---|---|---|---|
| ≦7 (3 + 4) | 26 | 150 | (50-260) | 0.004¶ |
| ≧7 (4 + 3) | 38 | 200 | (100-290) | |

Pathological stage†

| | | | | |
|---|---|---|---|---|
| pT₂-pT₃ₐ | 42 | 175 | (50-290) | 0.046¶ |
| pT_{3b}-pT_{any}pN₁ | 22 | 210 | (100-280) | |

PSA (ng/mL)†

| | | | | |
|---|---|---|---|---|
| <10 | 48 | 180 | (50-280) | NS¶ |
| ≧10 | 14 | 200 | (100-290) | |
| Androgen-independent | 10 | 250 | (80-300) | . . . |
| Metastatic prostate cancer | | | | |

Lymph nodes

| | | | | |
|---|---|---|---|---|
| Androgen-dependent | 4 | 235 | (200-290) | NS∥ |
| Androgen-independent | 8 | 235 | (190-300) | |
| Bone | 6 | 270 | (140-290) | . . . |

NS = non-significant.
*Categories 1+-3+ were used for evaluation of benign prostatic tissues and comparison to prostatic intraepithelial neoplasia and primary prostate cancer. A combined intensity per percentage of immunostained tumour cells scoring system was used to evaluate differences in expression among cancerous specimens (see text),
†Only the predominant tumour focus in each case was considered (64/71 cases).
§Wilcoxon signed rank test.
∥Mann-Whitney rank sum test.
¶Spearman correlation test.

B. Glucose Regulated Protein 78 (GRP 78)

Fingerprinting the repertoire of circulating antibodies from cancer patients using phage display libraries as a strategy for selection of targets in cancer has previously been described. Using this technique, the Glucose-regulated protein-78 (GRP78), a stress-responsive heat-shock protein involved in antigen presentation was described as a possible molecular marker for prostate cancer. Immune response against this protein was shown to have strong correlation with the development of androgen-independent prostate cancer and shorter overall survival. Thus, this protein has been targeted for diagnosis and/or treatment of prostate cancer.

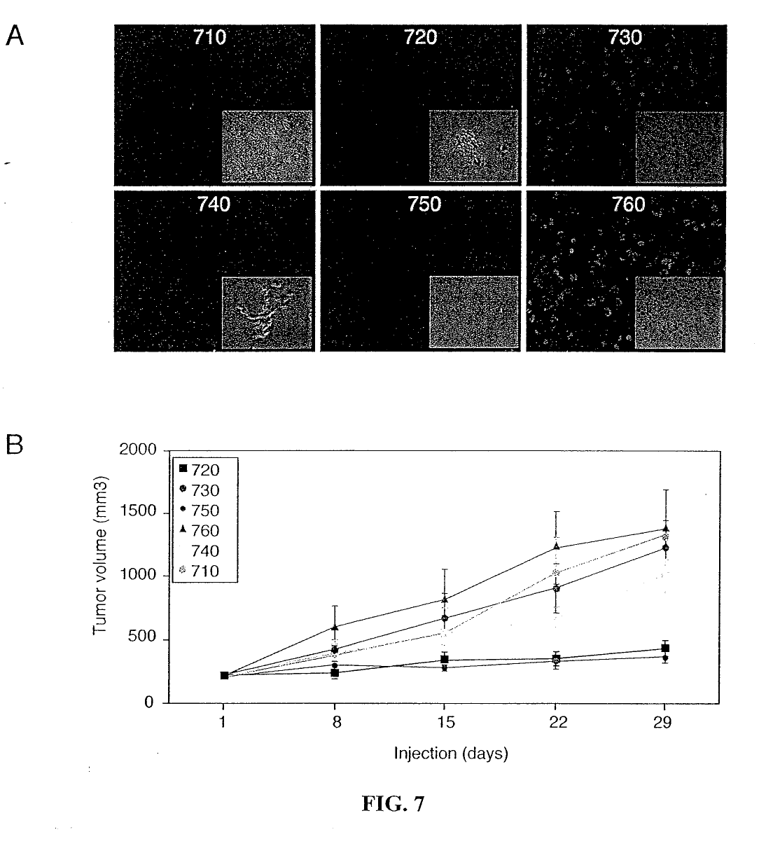

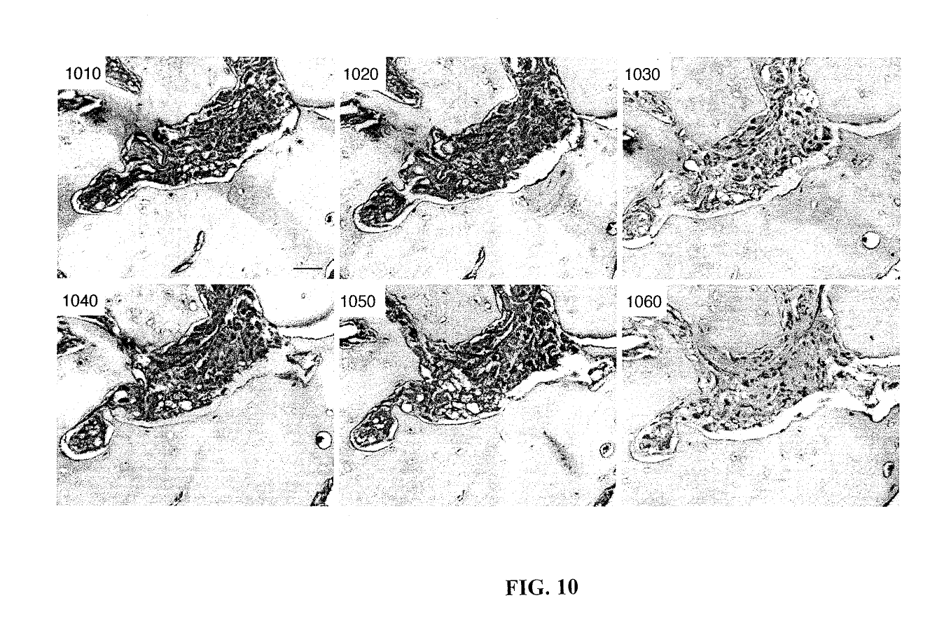

The presence of circulating antibodies against GRP78 was associated with the most aggressive stage of prostate cancer (metastatic androgen-independent disease). The expression of GRP78 was examined by immunohistochemical analysis in normal prostate tissue and bone marrow metastasis from a prostate cancer. The GRP78 antigen was highly expressed in bone marrow metastasis as shown by strong immunostaining (FIG. 10), whereas weak staining was observed in normal prostate tissue (FIG. 10). These results confirm the Western analysis using the same tissue samples noted above (FIG. 7). To show specificity, staining was inhibited using recombinant GRP78 (FIG. 10) or the peptide fusion protein (GST)-CNVSDKSC (SEQ ID NO:8) (FIG. 10). These data demonstrate that GRP78 is highly expressed in prostate cancer metastases to bone marrow and weakly expressed in normal prostate tissue.

One example shows that it is possible to identify molecular markers of disease progression and survival without prior knowledge of the antigens related to the disease. In cases where the tumor antigen is unknown, disease-specific antigens identified by this approach could be employed to define common or unique features in the immune response of individuals to the same disease, i.e., to fingerprint the immune response against a given antigen. The approach presented here is based on selection of immunoglobulin-binding peptides that mimic tumor-related antigens from phage libraries. Serum samples from human prostate cancer were screened and an antibody-binding peptide ligand was validated by using a large panel of patient serum samples. The corresponding tumor antigen eliciting the immune response was identified as GRP78, a molecular marker of use for detection, diagnosis and/or prognosis of metastatic prostate cancer. The GRP78 protein is highly expressed in bone marrow metastasis and the high prevalence of circulating antibodies against GRP78 is associated with metastatic androgen-independent disease and poor prognosis.

GRP78 (also known as Hsp70 protein 5) expression is induced by cellular stress and hypoxia, conditions associated with prostate cancer. Recently, this protein has been shown to be abundant in malignant prostate tumor by two-dimensional electrophoresis and mass spectrometry (Alaiya et al., 2001). In addition to GRP78, other heat shock proteins, such as 90, 72, and 27, are highly expressed in malignant prostate tissue (Thomas et al., 1996). GRP78 associates with the major histocompatibility complex (MHC) class I on the cell surface and its presence on the cell surface is not dependent on MHC class I expression (Triantafilou et al., 2001). Cancer-derived HSP-peptide complexes are being used as HSP vaccine in human cancer (Tamura et al., 1997). A recent study showed that the expression of heat shock proteins could independently determine the clinical outcome of individual prostate cancers (Tamura et al., 1997).

Although phage peptide libraries have been used to identify various pathological and disease-related agents in patients including Lyme disease, hepatitis, HIV-1, and autoimmune diseases, this is the first report in which sera from prostate cancer patients have been used to identify new markers for this cancer.

It is not unusual for tumor cells to shed antigens into the circulation. Leukocytes may also be exposed to tumor antigens in situ. It is therefore expected that cancer patients in general will exhibit circulating antibodies against tumor antigens. Phage display libraries may be screened against cancer patient samples to identify targeting peptides that bind to antibodies against tumor specific or tumor associated antigens. The identified targeting peptides may be used, for example, to purify anti-tumor antibodies using affinity chromatography or other well-known techniques. The purified anti-tumor antibodies can be used in diagnostic kits to identify individuals with cancer. Alternatively, they could be attached to various therapeutic moieties, such as chemotherapeutic agents, radioisotopes, anti-angiogenic agents, or pro-apoptosis agents and used for cancer therapy. The targeting peptides against anti-tumor antibodies may also be used to identify novel tumor specific or tumor-associated antigens, of diagnostic or therapeutic use. Phage display antibody libraries may also be constructed and screened against tumor targeting peptides. By this method, it is possible to isolate and purify large quantities of antibodies specific for tumor antigens.

Many malignant, cardiovascular, and inflammatory diseases have a marked angiogenic component. In cancer, tumor vasculature is a suitable target for intervention because the vascular endothelium is composed of non-malignant cells that are genetically stable but epigenetically diverse (St. Croix, 2000; Kolonin et al., 2001). In vivo phage display has been used to isolate probes that home selectively to different vascular beds and target receptors expressed only on certain blood vessels. Both tissue-specific and angiogenesis-related vascular ligand-receptor pairs have been identified with this technology. Targeted delivery of cytotoxic drugs (Arap et al., 1998a), proapoptotic peptides (Ellerby et al., 1999), fluorophores (Hong and Clayman, 2000) or cytokines (Curnis et al., 2000) to the vasculature generally improved selectivity and/or therapeutic windows in animal models. Vascular receptors are attractive targets for systemic delivery of gene therapy. Such receptors are readily accessible through the circulation and often can mediate internalization of ligands by cells (Kolonin et al., 2001).

While incorporation of vascular homing peptides derived from in vivo phage display screenings into viral vectors has been attempted, this strategy has proven quite challenging because the structure of the capsid and the targeting properties of the peptides can be adversely affected (Wickham, 2000). However, gene expression in mammalian cells is possible if phage vectors are processed in the correct trafficking pathway (Poul and Marks, 1999).

In theory, phage vectors have several advantages over mammalian viruses conventionally used for gene therapy. Receptors for prokaryotic viruses such as untargeted (wild-type) phage are not expressed on mammalian cells. Receptor-mediated internalization by mammalian cells does occur if re-targeted phage vectors display certain peptide ligands (Larocca et al., 1999). There is substantial evidence suggesting that phage can be safely administered to patients, as bacteriophage were given to humans during the pre-antibiotic era with no adverse effects (Barrow and Soothill, 1997). Because homing phage have been pre-selected to home to vascular receptors in an in vivo screening, there is no need for further targeting modifications. The localization of gene expression in vivo recapitulates previous observations using immunohistochemistry for phage localization (Rajotte et al., 1998; Rajotte and Ruoslahti, 1999; Pasqualini et al., 1997). The parental tumor-homing phage used in the Examples below are known to target receptors expressed in the activated blood vessels of multiple types of human and murine tumors, including carcinomas, melanomas, and sarcomas in mouse models (Pasqualini et al., 1997; Arap et al., 1998a; Koivunen et al., 1999a). The lung-homing phage and its corresponding receptor expressed in the lung vasculature have also been well characterized in mice (Rajotte et al., 1998; Rajotte and Ruoslahti, 1999).

Based on the rationale outlined above, targeted systemic gene delivery to the vascular endothelium may be accomplished with phage particles homing to cell surface receptors on blood vessels while meeting receptor requirements for selective tissue expression and vector accessibility. The results presented herein demonstrate the feasibility of this approach.

A new generation of targeted phage-based vectors is provided that enables systemic gene delivery and robust long-term transgene expression. A novel chimeric phage-based vector containing the inverted terminal repeat (ITR) sequences from adeno-associated virus (AAV) has been designed, constructed, and evaluated. These vectors (i) specifically home to receptors that have been well characterized for selective expression on the vascular endothelium, (ii) can deliver genes to angiogenic or tissue-specific blood vessels, and (iii) markedly increase transduction stability and duration of gene expression. These data indicate that targeted phage-based vectors and their derivatives are of use for clinical applications, such as targeted delivery to prostate cancer. In one embodiment, a phage-based vector may be used to deliver a targeting peptide to cancer tissue. In another embodiment, a phage-based vector may be used to deliver a targeting peptide complexed to an apoptotic agent to cancer tissue to induce apoptosis. Peptides selective for GRP78 include, but are not limited to WIFPWIQL (SEQ ID NO:6) and WDLAWMFRLPVG (SEQ ID NO:7)

II. Targeting Adipose Tissue

Obesity is an increasingly prevalent human condition in developed societies. Despite major progress in the understanding of the molecular mechanisms leading to obesity, no safe and effective treatment has yet been found. Diet and lifestyle contribute to the high incidence of obesity in the developed world. In the United States, approximately 65% of the adult population is overweight with a body mass index (BMI) of greater than or equal to 25 kg/m² and over 30% being obese (BMI of greater than or equal to 30 kg/m²). Obesity is associated with increased risk for diabetes mellitus, cancer, heart disease and it often causes shortening of human life. Advances in the treatment of obesity have thus far been rather limited with few drugs available to control abnormal fat accumulation.

Another difficult condition to target and treat is targeting fat tissue for weight loss for example directly targeting the adipose tissue. Peptides that target fat tissue may prove useful in treating the condition of obesity. Currently, methods for control of weight include dieting and surgical procedures. These often exhibit adverse effects and may not result in long-term weight loss. Dieting includes both popular (Fad) diets and the use of weight loss and appetite supplements. Fad diets are only good for short-term weight loss and do not achieve long-term weight control. They are often unhealthy, since many important nutrients are missing from the diet. In addition, rapid weight loss can result in dehydration.

Appetite suppressants such as Phentermen HCl, Meridia, Xernical, Adipex-P, Bontril and Ionomin may have adverse effects, such as addiction, dry mouth, nausea, irritability, and constipation. These supplements can also lead to more serious problems like eating disorders. Weight control through use of such supplements is ineffective, with only limited weight loss achieved. Effective drugs for controlling weight, such as fenfluramine, were withdrawn from the market due to cardiotoxicity.

Surgical methods for weight reduction, such as liposuction and gastric bypass surgery, have many risks. Liposuction removes subcutaneous fat through a suction tube inserted into a small incision in the skin. Risks and complications may include scarring, bleeding, infection, change in skin sensation, pulmonary complications, skin loss, chronic pain, etc. In gastric bypass surgery, the patient has to go through the rest of his or her life with a drastically altered stomach that can hold just two or three ounces of food. Side effects may include nausea, diarrhea, bleeding, infection, bowel blockage caused by scar tissue, hernia and adverse reactions to general anesthesia. The most serious potential risk is leakage of fluid from the stomach or intestines, which may result in abdominal infection and the need for a second surgery. None of the presently available methods for weight control is satisfactory and a need exists for improved methods of weight loss and control.

Another adipose related disease state is lipodystrophy syndrome(s) related to HIV infection (e.g., Jain et al., 2001). Mortality rates from HIV infection have decreased substantially following use of highly active antiretroviral therapy (HAART). However, treatment with protease inhibitors as part of the HAART protocol appears to result in a number of lipid-related symptoms, such as hyperlipidemia, fat redistribution with accumulation of abdominal and cervical fat, diabetes mellitus and insulin resistance (Raulin et al., 2002). Although of minor significance compared to the underlying HIV infection and possible development of AIDS related complex (ARC) and/or AIDS, lipodystrophy syndrome adversely affects quality of life and may be associated with increased risk of coronary artery disease, heart attack, stroke and other adverse side affects of increased blood lipids. While treatment with metformin, an insulin-sensitizing agent, has been reported to provide some alleviation of symptoms (Hadigan et al., 2000), a need exists for more effective methods of treating HIV related lipodystrophy.

Most anti-obesity agents are based on altering energy balance pathways and appetite by acting on receptors in the brain. Moreover, some drugs of this class (such as fenfluramine) have been withdrawn from the market due to unexpected toxicity. Recent attempts to develop compounds that inhibit absorption of fat through gastrointestinal tract (such as Orlistat) may improve anti-obesity treatment. Still, even the most effective drugs can only reduce weight by up to 5% and strict dieting is required for further weight loss.

Proliferation of tumor cells depends on new blood vessel formation (angiogenesis) that accompanies malignant progression. Anti-cancer therapy using angiogenesis inhibitors or cytotoxic agents targeted to the vasculature of tumors are currently being evaluated in as therapeutics in clinical trials. While white fat is a non-malignant tissue, it has the capability to quickly proliferate and expand similar to a tumor cell population. Histological evaluation of adipose tissue reveals that fat is highly vascularized similar to some tumor cell populations: multiple capillaries make contacts with every adipocyte, suggesting the importance of blood vessels for maintenance of the tissue mass. It was recently demonstrated that non-specific angiogenesis inhibitors may prevent the development of obesity in mice, and regulation of hepatic tissue mass by angiogenesis has also been reported. Targeting existing blood vessels in white fat may result in adipose tissue ablation. Peptide ligands were selected that bind to receptors in white fat vasculature. Targeted delivery of a chimeric peptide containing a pro-apoptotic sequence to the fat vasculature of obese mice was used that resulted in obesity reversal and metabolic normalization without change in food intake. In addition, prohibitin as the vascular receptor for one of the peptide ligands in white fat tissue was identified.

The invention provides additional compositions and methods for using targeting peptides selective and/or specific for adipose tissue, white adipose tissue, or placenta. In some embodiments, the invention concerns particular targeting peptides selective or specific for adipose or placental tissue, including but not limited to SEQ ID No 4, 9, and/or 10. Other embodiments concern such targeting peptides attached to therapeutic agents. In other embodiments, placental, adipose or other targeting peptides may be used to selectively or specifically deliver therapeutic agents to target tissues, such as white adipose tissue, placenta or fetal tissue. In certain embodiments, the subject methods concern the preparation and identification of targeting peptides selective or specific for a given target cell, tissue, or organ, such as adipose. Adipose targeting peptides include, but are not limited to CKGGRAKDC (SEQ ID NO:4), CARAC (SEQ ID NO:9), or CGDKAKGRC (SEQ ID NO:10).

III. Prostate Cancer Detection and Diagnosis

Carcinoma of the prostate (PCA) is the most frequently diagnosed cancer among men in the United States. Although relatively few prostate tumors progress to clinical significance during the lifetime of the patient, those that are progressive in nature are likely to have metastasized by the time of detection. Survival rates for individuals with metastatic prostate cancer are quite low. Between these extremes are patients with prostate tumors that will metastasize but have not yet done so, for whom surgical prostate removal is curative. Determination of which group a patient falls within is critical in determining optimal treatment and patient survival.

Serum prostate specific antigen (PSA) is widely used as a biomarker to detect and monitor therapeutic response in prostate cancer patients (Badalament et al., 1996; O'Dowd et al., 1997). Although PSA has been widely used since 1988 as a clinical marker of prostate cancer (Partin and Oesterling, 1994), screening programs utilizing PSA alone or in combination with digital rectal examination (DRE) have not been successful in improving the survival rate for men with prostate cancer (Partin and Oesterling, 1994). PSA is produced by normal and benign as well as malignant prostatic tissue, resulting in a high false-positive rate for prostate cancer detection (Partin and Oesterling, 1994). While an effective indicator of prostate cancer when serum levels are relatively high, PSA serum levels are more ambiguous indicators of prostate cancer when only modestly elevated. The specificity of the PSA assay for prostate cancer detection at low serum PSA levels remains a problem.

Other markers that have been used for prostate cancer detection include prostatic acid phosphatase (PAP) (Brawn et al., 1996), prostate secreted protein (PSP) (Huang et al., 1993), prostate specific membrane antigen (PSMA) (Murphy et al., 1996), human kallekrein 2 (HK2) (Piironen et al., 1996), prostate specific transglutaminase (pTGase) and interleukin 8 (IL-8) (Veltri et al., 1999). None of these has yet been demonstrated to provide a more sensitive and discriminating test for prostate cancer than PSA.

In addition to these protein markers for prostate cancer, genetic changes reported to be associated with prostate cancer, include allelic loss (Bova, et al., 1993); DNA hypermethylation (Isaacs et al., 1994); point mutations or deletions of the retinoblastoma (Rb), p53 and KAI1 genes (Isaacs et al., 1991); aneuploidy and aneusomy of chromosomes detected by fluorescence in situ hybridization (FISH) (Macoska et al., 1994) and differential expression of HER2/neu oncogene receptor (An et al., 1998). None of these has been reported to exhibit sufficient sensitivity and specificity to be useful as general screening tools for asymptomatic prostate cancer.

In current clinical practice, the serum PSA assay and digital rectal exam (DRE) is used to indicate which patients should have a prostate biopsy (Orozco et al., 1998). Histological examination of the biopsied tissue is used to make the diagnosis of prostate cancer. A need exists for a serological test that is sensitive enough to detect small and early stage prostate tumors, that also has sufficient specificity to exclude a greater portion of patients with noncancerous conditions such as BPH.

There remain deficiencies in the prior art with respect to the identification of markers linked with the progression of prostate cancer and the development of diagnostic methods to monitor disease progression. The identification of novel, prostate selective or specific markers that are differentially expressed in metastatic and/or non-metastatic prostate cancer, compared to non-malignant prostate tissue, would represent a major, unexpected advance for the diagnosis, prognosis and treatment of prostate cancer. As discussed below, one approach to identifying novel prostate cancer markers involves the phage display technique. The skilled artisan will realize that although various embodiments of the invention are discussed in terms of prostate cancer, the disclosed methods and/or compositions may be of use to identify markers (targeting peptides) for other types of cancer within the scope of the invention.

IV. Phage Display

Recently, an in vivo selection system was developed using phage display libraries to identify organ, tissue or cell type-targeting peptides in a mouse model system. Such libraries can be generated by inserting random oligonucleotides into cDNAs encoding a phage surface protein, generating collections of phage particles displaying unique peptides in as many as 10⁹ permutations. (Pasqualini and Ruoslahti, 1996; Arap et al, 1998a; 1998b).

Intravenous administration of phage display libraries to mice was followed by the recovery of phage from individual organs (Pasqualini and Ruoslahti, 1996). Phage were recovered that were capable of selective homing to the vascular beds of different mouse organs, tissues or cell types, based on the specific targeting peptide sequences expressed on the outer surface of the phage (Pasqualini and Ruoslahti, 1996). A variety of organ and tumor-homing peptides have been identified by this method (Rajotte et al., 1998; Rajotte et al, 1999; Koivunen et al., 1999a; Burg et al., 1999a; Pasqualini, 1999). Each of those targeting peptides bound to different receptors that were selectively expressed on the vasculature of the mouse target tissue (Pasqualini, 1999; Pasqualini et al., 2000; Folkman, 1997; Folkman, 1995). In addition to identifying individual targeting peptides selective for an organ, tissue or cell type (Pasqualini and Ruoslahti, 1996; Arap et al., 1998a; Koivunen et al., 1999b), this system has been used to identify endothelial cell surface markers that are expressed in mice in vivo (Rajotte and Ruoslahti, 1999).

Attachment of therapeutic agents to targeting peptides resulted in the selective delivery of the agent to a desired organ, tissue or cell type in the mouse model system. Targeted delivery of chemotherapeutic agents and proapoptotic peptides to receptors located in tumor angiogenic vasculature resulted in an increase in therapeutic efficacy and a decrease in systemic toxicity in tumor bearing mouse models (Arap et al., 1998a, 1998b; Ellerby et al., 1999).

The methods described herein for use of targeting peptides involve the in vivo discovery using phage display libraries. Various methods of phage display and methods for producing diverse populations of peptides are well known in the art. For example, U.S. Pat. Nos. 5,223,409; 5,622,699 and 6,068,829 disclose methods for preparing a phage library. The phage display technique involves genetically manipulating bacteriophage so that small peptides can be expressed on their surface (Smith and Scott, 1985, 1993). In addition to peptides, larger protein domains such as single-chain antibodies can also be displayed on the surface of phage particles (Arap et al., 1998a).

Targeting peptides selective for a given organ, tissue or cell type can be isolated by “biopanning” (Pasqualini and Ruoslahti, 1996; Pasqualini, 1999). In brief, a library of phage containing putative targeting peptides is administered to an animal or human and samples of organs, tissues or cell types containing phage are collected. In preferred embodiments utilizing filamentous phage, the phage may be propagated in vitro between rounds of biopanning in pilus-positive bacteria. The bacteria are not lysed by the phage but rather secrete multiple copies of phage that display a particular insert. Phage that bind to a target molecule can be eluted from the target organ, tissue or cell type and then amplified by growing them in host bacteria. If desired, the amplified phage can be administered to a host and samples of organs, tissues, or cell types again collected. Multiple rounds of biopanning can be performed until a population of selective binders is obtained. The amino acid sequence of the peptides is determined by sequencing the DNA corresponding to the targeting peptide insert in the phage genome. The identified targeting peptide can then be produced as a synthetic peptide by standard protein chemistry techniques (Arap et al., 1998a, Smith and Scott, 1985). This approach allows circulating targeting peptides to be detected in an unbiased functional assay, without any preconceived notions about the nature of their target. Once a candidate target is identified as the receptor of a targeting peptide, it can be isolated, purified and cloned by using standard biochemical methods (Pasqualini, 1999; Rajotte and Ruoslahti, 1999).

In certain embodiments, a subtraction protocol may be used with biopanning to further reduce background phage binding. The purpose of subtraction is to remove phage from the library that bind to cells other than the cell of interest, or that bind to inactivated cells. In alternative embodiments, the phage library may be prescreened against a subject who does not possess the targeted cell, tissue or organ. For example, prostate and/or prostate cancer binding peptides may be identified after prescreening a library against female subjects. After subtraction, the library may be screened against the cell, tissue or organ of interest. In another alternative embodiment, an unstimulated, quiescent cell type, tissue or organ may be screened against the library and binding phage removed. The cell line, tissue or organ is then activated, for example by administration of a hormone, growth factor, cytokine or chemokine and the activated cell type, tissue or organ screened against the subtracted phage library. Other methods of subtraction protocols are known and may be used in the practice of the present invention, for example as disclosed in U.S. Pat. Nos. 5,840,841, 5,705,610, 5,670,312 and 5,492,807, each of which is incorporated herein by references.

A. Choice of Phage Display System.

Previous in vivo selection studies performed in mice preferentially employed libraries of random peptides expressed as fusion proteins with the gene III capsule protein in the fuSE5 vector (Pasqualini and Ruoslahti, 1996). The number and diversity of individual clones present in a given library is a significant factor for the success of in vivo selection. It is preferred to use primary libraries, which are less likely to have an over-representation of defective phage clones (Koivunen et al., 1999b). The preparation of a library should be optimized to between 10⁸-10⁹ transducing units (T.U.)/ml. In certain embodiments, a bulk amplification strategy is applied between each round of selection.

Phage libraries displaying linear, cyclic, or double cyclic peptides may be used within the scope of the present invention. However, phage libraries displaying 3 to 10 random residues in a cyclic insert (CX₃₋₁₀C) are preferred, since single cyclic peptides tend to have a higher affinity for the target organ than linear peptides. Libraries displaying double-cyclic peptides (such as CX₃C X₃CX₃C; Rajotte et al., 1998) have been successfully used. However, the production of the cognate synthetic peptides, although possible, can be complex due to the multiple conformers with different disulfide bridge arrangements.

B. Identification of Homing Peptides and Receptors by In Vivo Phage Display in Mice

In vivo selection of peptides from phage-display peptide libraries administered to mice has been used to identify targeting peptides selective for normal mouse brain, kidney, lung, skin, pancreas, retina, intestine, uterus, prostate, and adrenal gland (Pasqualini and Ruoslahti, 1996; Pasqualini, 1999; Rajotte et al., 1998). These results show that the vascular endothelium of normal organs is sufficiently heterogeneous to allow differential targeting with peptide probes (Pasqualini and Ruoslahti, 1996; Rajotte et al., 1998). A panel of peptide motifs that target the blood vessels of tumor xenografts in nude mice has been assembled (Arap et al., 1998a; reviewed in Pasqualini, 1999). These motifs include the sequences RGD-4C, NGR, and GSL. The RGD-4C peptide has previously been identified as selectively binding αv integrins and has been reported to home to the vasculature of tumor xenografts in nude mice (Arap et al., 1998a, 1998b; Pasqualini et al., 1997).

Tumor-homing phage co-localize with their receptors in the angiogenic vasculature of tumors but not in non-angiogenic blood vessels in normal tissues (Arap et al., 1998b). Immunohistochemical evidence shows that vascular targeting phage bind to human tumor blood vessels in tissue sections (Pasqualini et al., 2000) but not to normal blood vessels. A negative control phage with no insert (fd phage) did not bind to normal or tumor tissue sections. The expression of the angiogenic receptors was evaluated in cell lines, in non-proliferating blood vessels and in activated blood vessels of tumors and other angiogenic tissues such as corpus luteum. Flow cytometry and immunohistochemistry showed that these receptors are expressed in a number of tumor cells and in activated HUVECs (data not shown). The angiogenic receptors were not detected in the vasculature of normal organs of mouse or human tissues.

The distribution of these receptors was analyzed by immunohistochemistry in tumor cells, tumor vasculature, and normal vasculature. Alpha v integrins, CD13, aminopeptidase A, NG2, and MMP-2/MMP-9—the known receptors in tumor blood vessels—are specifically expressed in angiogenic endothelial cells and pericytes of both human and murine origin. Angiogenic neovasculature expresses markers that are either expressed at very low levels or not at all in non-proliferating endothelial cells (not shown).

A peptide mimic of interleukin-11 (IL11) has been isolated from the prostate, and its tissue and molecular binding specificity to the interleukin-11 receptor alpha (IL11Rα) validated. Thus, several embodiments herein utilize a peptide to the IL-11Rα for targeting to the receptor to diagnose and/or treat prostate cancer.

C. Targeted Delivery

Peptides that home to tumor vasculature may be coupled to cytotoxic drugs or pro-apoptotic peptides to yield compounds that may be more effective and less toxic than the parental compounds in experimental models of mice bearing tumor xenografts (Arap et al., 1998a; Ellerby et al, 1999). The insertion of an RGD-4C peptide into a surface protein of an adenovirus has produced an adenoviral vector that may be of use for tumor targeted gene therapy (Arap et al., 1998b).

D. Microparticles and Delivery.

One embodiment of a composition suitable for the described method includes the use of a bioerodiable microparticle. The bioerodible microparticle may consist of a bioerodible polymer such as poly (lactide-co-glycolide). The composition of the bioerodible polymer is controlled to release the growth factor over a period of 1-2 weeks. It was previously demonstrated that biodegradable microparticles for example, poly (lactide-co-glycolide) were capable of controlled release of an oligonucleotide. These microparticles were prepared by the multiple emulsion-solvent evaporation technique. In order to increase the uptake of the oligonucleotide into the microparticles it was accompanied by polyethylenimine (PEI). The PEI also tended to make the microparticles more porous thus facilitating the delivery of the oligonucleotide out of the particles (De Rosa et al. 2002) In one preferred embodiment of a composition, the bioerodible microparticle may be a PLGA polymer 50:50 with carboxylic acid end groups. PLGA is a base polymer often used for controlled release of drugs and medical implant materials (i.e., anti-cancer drugs such as anti-prostate cancer agents). Two common delivery forms for controlled release include a microcapsule and a microparticle (e.g., a microsphere). The polymer and the agent are combined and usually heated to form the microparticle prior to delivery to the site of interest (Mitsui Chemicals, Inc). One embodiment, the bioerodible polymer harbors at least one peptide for release. In one embodiment, the PLGA polymer 50:50 with carboxylic acid end groups harbors at least one peptide for slow release. It is preferred that each microparticle may release at least 20 percent of its contents and more preferably around 90 percent of its contents. In one embodiment, the microparticle harboring at least one peptide will degrade slowly over time releasing the factor or release the factor immediately upon contact with the target region in order to rapidly expose the area to an agent and/or peptide. In another embodiment, the microparticles may be a combination of controlled-release microparticles and immediate release microparticles. A preferred rate of deposition of the delivered agent and/or peptide will vary depending on the condition of the subject undergoing treatment.

Another embodiment of a composition suitable for the described method includes the use of non-bioerodible microparticles that may harbor one or more of the aforementioned agents and/or peptide. The agent may be released from the microparticle by controlled-release or rapid release. The microparticles may be placed directly in the region. The non-bioerodible microparticle may consist of a non-bioerodible polymer such as an acrylic based microsphere for example a tris acryl microsphere (provided by Biosphere Medical). In one embodiment, non-bioerodiable microparticles may be used alone or in combination with another agent to treat a subject. In another embodiment, non-bioerodiable microparticles may be used alone or in combination with an agent to recruit an immune response. In addition, non-bioerodiable microparticles may be used alone or in combination with another agent to increase humoral or cellular responses.

In one embodiment, the treatment agent compositions suitable for reinforcement of the infarct zone are rendered resistant to phagocytosis by inhibiting opsonin protein absorption to the composition of the particles. In this regard, treatment agent compositions including sustained release carriers include particles having an average diameter up to about 10 microns are considered. In other situations, the particle size may range from about 1 mm to about 200 mm. The larger size particles may be considered in certain cases to avoid macrophage frustration and to avoid chronic inflammation in the treatment site. When needed, the particle size of up to 200 mm may be considered.

One method of inhibiting opsonization and subsequent rapid phagocytosis of treatment agents is to form a composition comprising a treatment agent disposed with a carrier for example a sustained release carrier and to coat the carrier with an opsonin inhibitor. One suitable opsonin-inhibitor includes polyethylene glycol (PEG) that creates a brush-like steric barrier to opsonization. PEG may alternatively be blended into the polymer constituting the carrier, or incorporated into the molecular architecture of the polymer constituting the carrier, as a copolymer, to render the carrier resistant to phagocytosis. Examples of preparing the opsonin-inhibited microparticles include the following.

For the encapsulation polymers, a blend of a polyalkylene glycol such as polyethylene glycol (PEG), polypropylene 1,2-glycol or polypropylene 1,3-glycol is co-dissolved with an encapsulating polymer in a common organic solvent during the carrier forming process. The percentage of PEG in the PEG/encapsulating polymer blend is between five percent and 60 percent by weight. Other hydrophilic polymers such as polyvinyl pyrolidone, polyvinyl alcohol, or polyoxyethylene-polyoxypropylene copolymers can be used in place of polyalkylene glycols, although polyalkylene glycols and more specifically, polyethylene glycol is generally preferred.

Alternatively, a diblock or triblock copolymer of an encapsulating polymer such as poly (L-lactide), poly (D,L-lactide), or poly (lactide-co-glycolide) with a polyalkylene glycol may be prepared. Diblocks can be prepared by: (i) reacting the encapsulating polymer with a monomethoxy polyakylene glycol such as PEG with one protected hydroxyl group and one group capable of reacting with the encapsulating polymer, (ii) by polymerizing the encapsulating polymer on to the monomethoxy polyalkylene glycol such as PEG with one protected group and one group capable of reacting with the encapsulating polymer; or (iii) by reacting the encapsulating polymer with a polyalkylene glycol such as PEG with amino functional termination. Triblocks can be prepared as described above using branched polyalkylene glycols with protection of groups that are not to react. Opsonization resistant carriers (microparticles/nanoparticles) can also be prepared using the techniques described above to form sustained-release carriers (microparticles/nanoparticles) with these copolymers.

A second way to inhibit opsonization is the biomimetic approach. For example, the external region of cell membrane, known as the “glycocalyx”, is dominated by glycoslylated molecules that prevent non-specific adhesion of other molecules and cells. Surfactant polymers consisting of a flexible poly (vinyl amine) backbone randomly-dextran and alkanoyl (hexanoyl or lauroyl) side chains which constrain the polymer backbone to lie parallel to the substrate. Hydrated dextran side chains protrude into the aqueous phase, creating a glycocalyx-like monolayer coating that suppresses plasma protein deposition on the foreign body surface. To mimic glycocalyx, glycocalyx-like molecules can be coated on the carriers (e.g., nanoparticles or microparticles) or blended into a polymer constituting the carrier to render the treatment agent resistant to phagocytosis. An alternate biomimetic approach is to coat the carrier with, or blend in phosphorylcholine, a synthetic mimetic of phosphatidylcholine, into the polymer constituting the carrier.

E. BRASIL

In preferred embodiments, separation of phage bound to the cells of a target organ, tissue or cell type from unbound phage is achieved using the BRASIL (Biopanning and Rapid Analysis of Soluble Interactive Ligands) technique (PCT Patent Application PCT/US01/28124 entitled, “Biopanning and Rapid Analysis of Selective Interactive Ligands (BRASIL)” by Arap et al., filed Sep. 7, 2001, incorporated herein by reference in its entirety). In BRASIL an organ, tissue or cell type is gently separated into cells or small clumps of cells that are suspended in an aqueous phase. The aqueous phase is layered over an organic phase of appropriate density and centrifuged. Cells attached to bound phage are pelleted at the bottom of the centrifuge tube, while unbound phage remain in the aqueous phase. BRASIL may be performed in an in vivo protocol, in which organs, tissues or cell types are exposed to a phage display library by intravenous administration, or by an ex vivo protocol, where the cells are exposed to the phage library in the aqueous phase before centrifugation. A non-limiting exemplary application of the BRASIL technique is disclosed in the Examples below.

F. Preparation of Large Scale Primary Libraries

In certain embodiments, primary phage libraries are amplified before injection into a subject. A phage library is prepared by ligating targeting peptide-encoding sequences into a phage vector, such as fUSE5. The vector is transformed into pilus negative host E. coli such as strain MC1061. The bacteria are grown overnight and then aliquots are frozen to provide stock for library production. Use of pilus negative bacteria avoids the bias in libraries that arises from differential infection of pilus positive bacteria by different targeting peptide sequences.

To freeze, bacteria are pelleted from two thirds of a primary library culture (5 liters) at 4000×g for 10 min. Bacteria are resuspended and washed twice with 500 ml of 10% glycerol in water, then frozen in an ethanol/dry ice bath and stored at −80° C.

For amplification, 1.5 ml of frozen bacteria are inoculated into 5 liters of LB medium with 20 μg/ml tetracycline and grown overnight. Thirty minutes after inoculation, a serial dilution is plated on LB/tet plates to verify the viability of the culture. If the number of viable bacteria is less than 5-10 times the number of individual clones in the library (1-2×10⁸) the culture is discarded.

After growing the bacterial culture overnight, phage are precipitated. About ¼ to ⅓ of the bacterial culture is kept growing overnight in 5 liters of fresh medium and the cycle is repeated up to 5 times. Phage are pooled from all cycles and used for injection into human subjects.

V. Human Subjects

The methods used for phage display biopanning in the mouse model system require substantial improvements for use with humans. Techniques for biopanning in human subjects are disclosed in PCT Patent Application PCT/US01/28044, filed Sep. 7, 2001, the entire text of which is incorporated herein by reference. In general, humans suitable for use with phage display are either brain dead or terminal wean patients. The amount of phage library (preferably primary library) required for administration must be significantly increased, preferably to 10¹⁴ TU or higher, preferably administered intravenously in approximately 200 ml of Ringer lactate solution over about a 10 minute period.

The amount of phage required for use in humans has required substantial improvement of the mouse protocol, increasing the amount of phage available for injection by five orders of magnitude. To produce such large phage libraries, the transformed bacterial pellets recovered from up to 500 to 1000 transformations are amplified up to 10 times in the bacterial host, recovering the phage from each round of amplification and adding LB Tet medium to the bacterial pellet for collection of additional phage. The phage inserts remain stable under these conditions and phage may be pooled to form the large phage display library required for humans.

Samples of various organs and tissues are collected starting approximately 15 minutes after injection of the phage library. Samples are processed as described below and phage collected from each organ, tissue or cell type of interest for DNA sequencing to determine the amino acid sequences of targeting peptides.

A. Polyorgan Targeting

In the standard protocol for phage display biopanning, phage from a single organ are collected, amplified and injected into a new host, where tissue from the same organ is collected for phage rescue and a new round of biopanning.

It is possible to pool phage collected from multiple organs after a first round of biopanning and inject the pooled sample into a new subject, where each of the multiple organs may be collected again for phage rescue. The polyorgan targeting protocol may be repeated for as many rounds of biopanning as desired. In this manner, it is possible to significantly reduce the number of subjects required for isolation of targeting peptides for multiple organs, while still achieving substantial enrichment of the organ-homing phage.

In certain embodiments, phage are recovered from human organs, tissues or cell types after injection of a phage display library into a human subject. In certain embodiments, phage may be recovered by exposing a sample of the organ, tissue or cell type to a pilus positive bacterium, such as E. Coli K91. In alternative embodiments, phage may be recovered by amplifying the phage inserts, ligating the inserts to phage DNA and producing new phage from the ligated DNA.

VI. Proteins and Peptides

In certain embodiments, the present invention concerns novel compositions comprising at least one protein or peptide. As used herein, a protein or peptide generally refers, but is not limited to, a protein of greater than about 200 amino acids up to a full length sequence translated from a gene; a polypeptide of about 100 to 200 amino acids; and/or a peptide of from about 3 to about 100 amino acids. For convenience, the terms “protein,” “polypeptide” and “peptide are used interchangeably herein.

In certain embodiments the size of at least one protein or peptide may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues.

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid, including but not limited to those shown on Table 2 below.

TABLE 2
Modified and Unusual Amino Acids

| | | | |
|---|---|---|---|
| Abbr. | Amino Acid | Abbr. | Amino Acid |

| | | | |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino-propionic acid | AHyl | allo-Hydroxylysine |
| Abu | 2-Aminobutyric acid | 3Hyp | 3-Hydroxyproline |
| 4Abu | 4-Aminobutyric acid, piperidinic acid | 4Hyp | 4-Hydroxyproline |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

Proteins or peptides may be made by any technique known to those of skill in the art, including the expression of proteins, polypeptides, or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases (www.ncbi.nlm.nih.gov/). The coding regions for known genes may be amplified and/or expressed using the techniques disclosed herein or as would be know to those of ordinary skill in the art. Alternatively, various commercial preparations of proteins, polypeptides and peptides are known to those of skill in the art.

A. Peptide Mimetics

Another embodiment for the preparation of polypeptides according to the invention is the use of peptide mimetics. Mimetics are peptide-containing molecules that mimic elements of protein secondary structure. See, for example, Johnson et al., (1993), incorporated herein by reference. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting peptides disclosed herein, but with altered and even improved characteristics.

B. Fusion Proteins

Other embodiments of the present invention concern fusion proteins. These molecules generally have all or a substantial portion of a targeting peptide, linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In preferred embodiments, the fusion proteins of the instant invention comprise a targeting peptide linked to a therapeutic protein or peptide. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. These examples are not meant to be limiting and it is contemplated that within the scope of the present invention virtually and protein or peptide could be incorporated into a fusion protein comprising a targeting peptide. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

C. Protein Purification

In certain embodiments a protein or peptide may be isolated or purified. In one embodiment, these proteins may be used to generate antibodies for tagging with any of the illustrated barcodes (eg. polymeric Raman label). Protein purification techniques are well known to those of skill in the art. These techniques involve, at one level, the homogenization and crude fractionation of the cells, tissue or organ to polypeptide and non-polypeptide fractions. The protein or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, HPLC (high performance liquid chromatography) FPLC (AP Biotech), polyacrylamide gel electrophoresis, affinity chromatography, immunoaffinity chromatography and isoelectric focusing. An example of receptor protein purification by affinity chromatography is disclosed in U.S. Pat. No. 5,206,347, the entire text of which is incorporated herein by reference. One of the more efficient methods of purifying peptides is fast performance liquid chromatography (AKTA FPLC) or even A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand.

D. Synthetic Peptides

Because of their relatively small size, the targeting peptides of the invention can be synthesized in solution or on a solid support in accordance with conventional techniques. Various automatic synthesizers are commercially available and can be used in accordance with known protocols. See, for example, Stewart and Young, 1984; Tam et al., 1983; Merrifield, 1986; and Barany and Merrifield, 1979, each incorporated herein by reference. Short peptide sequences, usually from about 6 up to about 35 to 50 amino acids, can be readily synthesized by such methods. Alternatively, recombinant DNA technology may be employed wherein a nucleotide sequence which encodes a peptide of the invention is inserted into an expression vector, transformed or transfected into an appropriate host cell, and cultivated under conditions suitable for expression.

E. Antibodies

In certain embodiments, it may be desirable to make antibodies against the identified targeting peptides or their receptors. The appropriate targeting peptide or receptor, or portions thereof, may be coupled, bonded, bound, conjugated, or chemically-linked to one or more agents via linkers, polylinkers, or derivatized amino acids. This may be performed such that a bispecific or multivalent composition or vaccine is produced. It is further envisioned that the methods used in the preparation of these compositions are familiar to those of skill in the art and should be suitable for administration to humans, i.e., pharmaceutically acceptable. Preferred agents are the carriers are keyhole limpet hemocyanin (KLH) or bovine serum albumin (BSA).

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (See, e.g., Harlow and Lane, 1988; incorporated herein by reference).

In various embodiments of the invention, circulating antibodies from one or more individuals with a disease state may be obtained and screened against phage display libraries. Targeting peptides that bind to the circulating antibodies may act as mimeotopes of a native antigen, such as a receptor protein located on an endothelial cell surface of a target tissue. For example, circulating antibodies in an individual with prostate cancer may bind to antigens specifically or selectively localized in prostate tumors. As discussed in more detail below, targeting peptides against such antibodies may be identified by phage display. Such targeting peptides may be used to identify the native antigen recognized by the antibodies, for example by using known techniques such as immunoaffinity purification, Western blotting, electrophoresis followed by band excision and protein/peptide sequencing and/or computerized homology searches. The skilled artisan will realize that antibodies against disease specific or selective antigens may be of use for various applications, such as detection, diagnosis and/or prognosis of a disease state, imaging of diseased tissues and/or targeted delivery of therapeutic agents.

F. Imaging Agents and Radioisotopes

In certain embodiments, the claimed peptides or proteins of the present invention may be attached to imaging agents of use for imaging and diagnosis of various diseased organs, tissues or cell types. For example, a prostate cancer selective targeting peptide may be attached to an imaging agent, provided to a subject and the precise boundaries of the cancer tissue may be determined by standard imaging techniques, such as CT scanning, MRI, PET scanning, etc. Alternatively, the presence or absence and location in the body of metastatic prostate cancer may be determined by imaging using one or more targeting peptides that are selective for metastatic prostate cancer. Targeting peptides that bind to normal as well as cancerous prostate tissues may still be of use, as such peptides would not be expected to be selectively localized anywhere besides the prostate in disease-free individuals. Naturally, the distribution of a prostate or prostate cancer selective targeting peptide may be compared to the distribution of one or more non-selective peptides to provide even greater discrimination for detection and/or localization of diseased tissues.

Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III).

Radioisotopes of potential use as imaging or therapeutic agents include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being preferred for use in certain embodiments, and technicium^{99m} and indium¹¹¹ are also often preferred due to their low energy and suitability for long range detection.

Radioactively labeled proteins or peptides of the present invention may be produced according to well-known methods in the art. For instance, they can be iodinated by contact with sodium or potassium iodide and a chemical oxidizing agent such as sodium hypochlorite, or an enzymatic oxidizing agent, such as lactoperoxidase. Proteins or peptides according to the invention may be labeled with technetium-^{99m} by ligand exchange process, for example, by reducing pertechnate with stannous solution, chelating the reduced technetium onto a Sephadex column and applying the peptide to this column or by direct labeling techniques, e.g., by incubating pertechnate, a reducing agent such as SNCl₂, a buffer solution such as sodium-potassium phthalate solution, and the peptide. Intermediary functional groups that are often used to bind radioisotopes that exist as metallic ions to peptides are diethylenetriaminepenta-acetic acid (DTPA) and ethylene diaminetetra-acetic acid (EDTA). Also contemplated for use are fluorescent labels, including rhodamine, fluorescein isothiocyanate and renographin.

In certain embodiments, the claimed proteins or peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

G. Cross-Linkers

The targeting peptides, ligands, receptor proteins and other molecules of interest may be attached to surfaces or to therapeutic agents and other molecules using a variety of known cross-linking agents. Methods for covalent or non-covalent attachment of proteins or peptides are well known in the art. Such methods may include, but are not limited to, use of chemical cross-linkers, photoactivated cross-linkers and/or bifunctional cross-linking reagents. Exemplary methods for cross-linking molecules are disclosed in U.S. Pat. Nos. 5,603,872 and 5,401,511, incorporated herein by reference. Non-limiting examples of cross-linking reagents of potential use include glutaraldehyde, bifunctional oxirane, ethylene glycol diglycidyl ether, carbodiimides such as 1-ethyl-3-(3-dimethylaminopropyl) carbodiimide or dicyclohexylcarbodiimide, bisimidates, dinitrobenzene, N-hydroxysuccinimide ester of suberic acid, disuccinimidyl tartarate, dimethyl-3,3′-dithio-bispropionimidate, azidoglyoxal, N-succinimidyl-3-(2-pyridyldithio)propionate and 4-(bromoadminoethyl)-2-nitrophenylazide.

Homobifunctional reagents that carry two identical functional groups are highly efficient in inducing cross-linking. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied.

In certain embodiments, it may be appropriate to link one or more targeting peptides to a liposome or other membrane-bounded particle. For example, targeting peptides cross-linked to liposomes, microspheres or other such devices may be used to deliver larger volumes of a therapeutic agent to a target organ, tissue or cell type. Various ligands can be covalently bound to liposomal surfaces through the cross-linking of amine residues. Liposomes containing phosphatidylethanolamine (PE) may be prepared by established procedures. The inclusion of PE provides an active functional amine residue on the liposomal surface.

In another non-limiting example, heterobifunctional cross-linking reagents and methods of use are disclosed in U.S. Pat. No. 5,889,155, incorporated herein by reference. The cross-linking reagents combine a nucleophilic hydrazide residue with an electrophilic maleimide residue, allowing coupling in one example, of aldehydes to free thiols. The cross-linking reagent can be modified to cross-link various functional groups.

Other techniques of general use for proteins or peptides that are known in the art have not been specifically disclosed herein, but may be used in the practice of the claimed subject matter.

VII. Nucleic Acids

In certain embodiments, nucleic acids may encode a targeting peptide, a receptor protein, a fusion protein or other protein or peptide. The nucleic acid may be derived from genomic DNA, complementary DNA (cDNA) or synthetic DNA. Where incorporation into an expression vector is desired, the nucleic acid may also comprise a natural intron or an intron derived from another gene. Such engineered molecules are sometime referred to as “mini-genes.” In various embodiments of the invention, targeting peptides may be incorporated into gene therapy vectors via nucleic acids.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of almost any size, determined in part by the length of the encoded protein or peptide.

It is contemplated that targeting peptides, fusion proteins and receptors may be encoded by any nucleic acid sequence that encodes the appropriate amino acid sequence. The design and production of nucleic acids encoding a desired amino acid sequence is well known to those of skill in the art, using standardized codon tables. In preferred embodiments, the codons selected for encoding each amino acid may be modified to optimize expression of the nucleic acid in the host cell of interest. Codon preferences for various species of host cell are well known in the art.

In addition to nucleic acids encoding the desired peptide or protein, the present invention encompasses complementary nucleic acids that hybridize under high stringency conditions with such coding nucleic acid sequences. High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture.

Nucleic acids for use in the disclosed methods and compositions may be produced by any method known in the art, such as chemical synthesis (e.g. Applied Biosystems Model 3900, Foster City, Calif.), purchase from commercial sources (e.g. Midland Certified Reagents, Midland, Tex.) and/or standard gene cloning methods. A number of nucleic acid vectors, such as expression vectors and/or gene therapy vectors, may be commercially obtained (e.g., American Type Culture Collection, Rockville, Md.; Promega Corp., Madison, Wis.; Stratagene, La Jolla, Calif.).

A. Vectors for Cloning, Gene Transfer and Expression

In certain embodiments expression vectors are employed to express the targeting peptide or fusion protein, which can then be purified and used. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are known.

B. Regulatory Elements

The terms “expression construct” or “expression vector” are meant to include any type of genetic construct containing a nucleic acid coding for a gene product in which part or all of the nucleic acid coding sequence is capable of being transcribed. In preferred embodiments, the nucleic acid encoding a gene product is under transcriptional control of a promoter. A “promoter” refers to a DNA sequence recognized by the synthetic machinery of the cell, or introduced synthetic machinery, required for initiating the specific transcription of a gene. The phrase “under transcriptional control” means that the promoter is in the correct location and orientation in relation to the nucleic acid to control RNA polymerase initiation and expression of the gene.

In various embodiments, the human cytomegalovirus (CMV) immediate early gene promoter, the SV40 early promoter, the Rouse sarcoma virus long terminal repeat, rat insulin promoter, and glyceraldehyde-3-phosphate dehydrogenase promoter can be used to obtain high-level expression of the coding sequence of interest. The use of other viral or mammalian cellular or bacterial phage promoters that are known in the art to achieve expression of a coding sequence of interest is contemplated as well, provided that the levels of expression are sufficient for a given purpose.

Where a cDNA insert is employed, one will typically include a polyadenylation signal to effect proper polyadenylation of the gene transcript. The nature of the polyadenylation signal is not believed to be crucial to the successful practice of the invention, and any such sequence may be employed, such as human growth hormone and SV40 polyadenylation signals. Also contemplated as an element of the expression construct is a terminator. These elements can serve to enhance message levels and to minimize read through from the construct into other sequences.

C. Selectable Markers

In certain embodiments of the invention, the cells containing nucleic acid constructs of the present invention may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin, and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

D. Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome, and express viral genes stably and efficiently have made them attractive candidates for the transfer of foreign genes into mammalian cells (Ridgeway, 1988; Nicolas and Rubinstein, 1988.; Baichwal and Sugden, 1986; Temin, 1986). Preferred gene therapy vectors are generally viral vectors.

In using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

DNA viruses used as gene vectors include the papovaviruses (e.g., simian virus 40, bovine papilloma virus, and polyoma) (Ridgeway, 1988; Baichwal and Sugden, 1986) and adenoviruses (Ridgeway, 1988; Baichwal and Sugden, 1986).

An exemplary method for in vivo delivery involves the use of an adenovirus expression vector. Although adenovirus vectors have a low capacity for integration into genomic DNA, this feature is counterbalanced by the high efficiency of gene transfer afforded by these vectors. “Adenovirus expression vector” is meant to include, but is not limited to, constructs containing adenovirus sequences sufficient to (a) support packaging of the construct and (b) to express an antisense or a sense polynucleotide that has been cloned therein.

Generation and propagation of adenovirus vectors that are replication deficient depend on a helper cell line, such as the 293 cell line, which was transformed from human embryonic kidney cells by Ad5 DNA fragments and constitutively expresses E1 proteins (Graham et al., 1977.). Since the E3 region is dispensable from the adenovirus genome (Jones and Shenk, 1978), adenovirus vectors, with the help of 293 cells, carry foreign DNA in either the E1, the E3, or both regions (Graham and Prevec, 1991.).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. Racher et al., (1995) disclosed methods for culturing 293 cells and propagating adenovirus.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic innoculation into the brain (Le Gal La Salle et al., 1993). In preferred embodiments, gene therapy vectors are based upon adeno-associated virus (AAV).

Other gene transfer vectors may be constructed from retroviruses. (Coffin, 1990.) The retroviral genome contains three genes, gag, pol, and env. that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences, and also are required for integration in the host cell genome (Coffin, 1990).

In order to construct a retroviral vector, a nucleic acid encoding protein of interest is inserted into the viral genome in the place of certain viral sequences to produce a virus that is replication-defective. In order to produce virions, a packaging cell line containing the gag, pol, and env genes, but without the LTR and packaging components, is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are capable of infecting a broad variety of cell types. However, integration and stable expression require the division of host cells (Paskind et al., 1975).

Other viral vectors may be employed as expression constructs. Vectors derived from viruses such as vaccinia virus (Ridgeway, 1988; Baichwal and Sugden, 1986; Coupar et al., 1988), adeno-associated virus (AAV) (Ridgeway, 1988; Baichwal and Sugden, 1986; Hermonat and Muzycska, 1984), and herpes viruses may be employed. They offer several attractive features for various mammalian cells (Friedmann, 1989; Ridgeway, 1988; Baichwal and Sugden, 1986; Coupar et al., 1988; Horwich et al., 1990).

Several non-viral methods for the transfer of expression constructs into cultured mammalian cells also are contemplated. These include calcium phosphate precipitation (Graham and van der Eb, 1973.; Chen and Okayama, 1987.; Rippe et al., 1990; DEAE dextran (Gopal, et al. 1985), electroporation (Tur-Kaspa et al., 1986; Potter et al., 1984), direct microinjection, DNA-loaded liposomes and lipofectamine-DNA complexes, cell sonication, gene bombardment using high velocity microprojectiles, and receptor-mediated transfection (Wu and Wu, 1987; Wu and Wu, 1988). Some of these techniques may be successfully adapted for in vivo or ex vivo use.

In a further embodiment of the invention, the expression construct may be entrapped in a liposome. Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa, and hepatoma cells. Nicolau et al., (1987.) accomplished successful liposome-mediated gene transfer in rats after intravenous injection.

VIII. Pharmaceutical Compositions

Where clinical applications are contemplated, it may be necessary to prepare pharmaceutical compositions—expression vectors, virus stocks, proteins, antibodies and drugs—in a form appropriate for the intended application. Generally, this will entail preparing compositions that are essentially free of impurities that could be harmful to humans or animals.

One generally will desire to employ appropriate salts and buffers to render delivery vectors stable and allow for uptake by target cells. Aqueous compositions of the present invention may comprise an effective amount of a protein, peptide, fusion protein, recombinant phage and/or expression vector, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the proteins or peptides of the present invention, its use in therapeutic compositions is contemplated. Supplementary active ingredients also can be incorporated into the compositions.

The active compositions of the present invention may include classic pharmaceutical preparations. Administration of these compositions according to the present invention are via any common route so long as the target tissue is available via that route. This includes oral, nasal, buccal, rectal, vaginal or topical. Alternatively, administration may be by orthotopic, intradermal, subcutaneous, intramuscular, intraperitoneal, intraarterial or intravenous injection. Such compositions normally would be administered as pharmaceutically acceptable compositions, described supra.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it is preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

IX. Therapeutic Agents

In certain embodiments, therapeutic agents may be attached to a targeting peptide or fusion protein for selective delivery to, for example, non-metastatic and/or metastatic prostate cancer. Agents or factors suitable for use may include any chemical compound that induces apoptosis, cell death, cell stasis and/or anti-angiogenesis or otherwise affects the survival and/or growth rate of a cancer cell.

A. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Tsujimoto et al., 1985). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins that share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

Non-limiting examples of pro-apoptosis agents contemplated within the scope of the present invention include gramicidin, magainin, mellitin, defensin, cecropin, (KLAKLAK)₂ (SEQ ID NO:11).

B. Angiogenic Inhibitors

In certain embodiments the present invention may concern administration of targeting peptides attached to anti-angiogenic agents, such as angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline.

Proliferation of tumors cells relies heavily on extensive tumor vascularization, which accompanies cancer progression. Thus, inhibition of new blood vessel formation with anti-angiogenic agents and targeted destruction of existing blood vessels have been introduced as an effective and relatively non-toxic approach to tumor treatment. (Arap et al., 1998a; 1998b; Ellerby et al., 1999). A variety of anti-angiogenic agents and/or blood vessel inhibitors are known. (e.g., Folkman, 1997; Eliceiri and Cheresh, 2001).

C. Cytotoxic Agents

A wide variety of anticancer agents are well known in the art and any such agent may be coupled to a cancer targeting peptide for use within the scope of the present invention. Exemplary cancer chemotherapeutic (cytotoxic) agents of potential use include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and “Remington: The Science and Practice of Pharmacy,” 20th edition, Gennaro, Lippincott, 2000, each incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Of course, all of these dosages and agents described herein are exemplary rather than limiting, and other doses or agents may be used by a skilled artisan for a specific patient or application. Any dosage in-between these points, or range derivable therein is also expected to be of use in the invention.

D. Alkylating Agents

Alkylating agents are drugs that directly interact with genomic DNA to prevent cells from proliferating. This category of chemotherapeutic drugs represents agents that affect all phases of the cell cycle, that is, they are not phase-specific. An alkylating agent, may include, but is not limited to, nitrogen mustard, ethylenimene, methylmelamine, alkyl sulfonate, nitrosourea or triazines. They include but are not limited to: busulfan, chlorambucil, cisplatin, cyclophosphamide (cytoxan), dacarbazine, ifosfamide, mechlorethamine (mustargen), and melphalan.

E. Antimetabolites

Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

F. Natural Products

Natural products generally refer to compounds originally isolated from a natural source (eg. herbal compositions), and identified as having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

G. Antibiotics

Certain antibiotics have both antimicrobial and cytotoxic activity. These drugs also interfere with DNA by chemically inhibiting enzymes and mitosis or altering cellular membranes. These agents are not phase specific so they work in all phases of the cell cycle. Examples of cytotoxic antibiotics include, but are not limited to, bleomycin, dactinomycin, daunorubicin, doxorubicin (Adriamycin), plicamycin (mithramycin) and idarubicin.

H. Miscellaneous Agents

Miscellaneous cytotoxic agents that do not fall into the previous categories include, but are not limited to, platinum coordination complexes, anthracenediones, substituted ureas, methyl hydrazine derivatives, amsacrine, L-asparaginase, and tretinoin. Platinum coordination complexes include such compounds as carboplatin and cisplatin (cis-DDP). An exemplary anthracenedione is mitoxantrone. An exemplary substituted urea is hydroxyurea. An exemplary methyl hydrazine derivative is procarbazine (N-methylhydrazine, MIH). These examples are not limiting and it is contemplated that any known cytotoxic, cytostatic or cytocidal agent may be attached to targeting peptides and administered to a targeted organ, tissue or cell type within the scope of the invention.

I. Cytokines and Chemokines

In certain embodiments, it may be desirable to couple specific bioactive agents to one or more targeting peptides for targeted delivery to an organ, tissue or cell type. Such agents include, but are not limited to, cytokines and/or chemokines.

The term “cytokine” is a generic term for proteins released by one cell population that act on another cell as intercellular mediators. Examples of cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-alpha. and -beta; mullerian-inhibiting substance; mouse gonadotropin-associated peptide; inhibin; activin; vascular endothelial growth factor; integrin; thrombopoietin (TPO); nerve growth factors such as NGF-.beta.; platelet-growth factor; transforming growth factors (TGFs) such as TGF-.alpha. and TGF-.beta.; insulin-like growth factor-I and -II; erythropoietin (EPO); osteoinductive factors; interferons such as interferon-α, -.β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1.alpha., IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand or FLT-3, angiostatin, thrombospondin, endostatin, tumor necrosis factor and LT. As used herein, the term cytokine includes proteins from natural sources or from recombinant cell culture and biologically active equivalents of the native sequence cytokines.

Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Chemokines include, but are not limited to, RANTES, MCAF, MIP1-alpha, MIP1-Beta, and IP-10. The skilled artisan will recognize that certain cytokines are also known to have chemoattractant effects and could also be classified under the term chemokines.

J. Dosages

The skilled artisan is directed to “Remington: The Science and Practice of Pharmacy,” (2000). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA Office of Biologics standards.

X. Screening Phage Libraries by Palm

In certain embodiments, it is desirable to be able to select specific cell types from a heterogeneous sample of an organ or tissue. One method to accomplish such selective sampling is by PALM (Positioning and Ablation with Laser Microbeams).

The PALM Robot-Microbeam uses a precise, computer-guided laser for microablation. A pulsed ultra-violet (UV) laser is interfaced into a microscope and focused through an objective to a beam spot size of less than 1 micrometer in diameter. The principle of laser cutting is a locally restricted ablative photodecomposition process without heating (Hendrix, 1999). The effective laser energy is concentrated on the minute focal spot only and most biological objects are transparent for the applied laser wavelength. This system appears to be the tool of choice for recovery of homogeneous cell populations or even single cells or subcellular structures for subsequent phage recovery. Tissue samples may be retrieved by circumcising a selected zone or a single cell after phage administration to the subject. A clear-cut gap between selected and non-selected area is typically obtained. The isolated tissue specimen can be ejected from the object plane and catapulted directly into the cap of a common micro centrifuge tube in an entirely non-contact manner. The basics of this so called Laser Pressure Catapulting (LPC) method is believed to be the laser pressure force that develops under the specimen, caused by the extremely high photon density of the precisely focused laser microbeam. This tissue harvesting technique allows the phage to survive the microdissection procedure and be rescued.

PALM was used in the present example to select targeting phage for mouse pancreatic tissue, as described below.

XI. Kits

In still further embodiments, the present invention concerns kits for use with the therapeutic and diagnostic methods described above. As the encoded proteins or peptides may be employed to target delivery of a therapeutic to a cell, and/or to detect antibodies or the corresponding antibodies may be employed to detect encoded proteins or peptides, either or both of such components may be provided in the kit. The immunodetection kits will thus comprise, in suitable container means, a protein or peptide or a nucleic acid encoding such, or a first antibody that binds to an encoded protein or peptide, and an immunodetection reagent.

In certain embodiments, the protein or peptide, or the first antibody that binds to the encoded protein or peptide, may be bound to a solid support, such as a column matrix or well of a microtiter plate.

Immunodetection reagents of the kit may take any one of a variety of forms, including those detectable labels that are associated with or linked to the given antibody or antigen, and detectable labels that are associated with or attached to a secondary binding ligand. Exemplary secondary ligands are those secondary antibodies that have binding affinity for the first antibody or antigen, and secondary antibodies that have binding affinity for a human antibody.

Further suitable immunodetection reagents for use in the present kits include the two-component reagent that comprises a secondary antibody that has binding affinity for the first antibody or antigen, along with a third antibody that has binding affinity for the second antibody, the third antibody being linked to a detectable label.

The kits may further comprise a suitably aliquoted composition of the encoded protein or peptide, whether labeled or unlabeled, as may be used to prepare a standard curve for a detection assay.

The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the peptide, peptide conjugate, antibody or antigen may be placed, and preferably, suitably aliquoted. Where a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Targeting Tumor Cells Using Selective Peptide Binding

A. Materials and Methods

Tissue Specimens and Immunohistochemistry. Ninety-nine formalin-fixed, paraffin-embedded human primary and metastatic prostate cancer samples were studied, derived from 90 patients (1 sample in 81 patients and 2 samples in 9 patients; median age: 61, range 40-81). Samples consisted of 81 primary adenocarcinomas, obtained either from radical prostatectomy (n=71 androgen-dependent, n=3 androgen-independent), cystoprostatectomy (n=6 androgen-independent), or pelvic exenteration (n=1 androgen-independent); and 18 lymph node and bone metastases (Table 3, which represents clinical and histopathological characteristics and IL11Rα expression). Human samples were selected to reflect: (i) stages in prostate cancer progression; (ii) differing Gleason scores; and (iii) zonal origin (peripheral zone and transition zone). Additional blocks from the same specimens, including benign prostatic tissues from peripheral (n=62), transition (n=51), and central zone (n=40), were included.

Tissue samples were stained within two weeks of sectioning. Four μm sections were antigen-retrieved by heat with EDTA (pH 8.0; Zymed, San Francisco, Calif.), and biotin and protein blocked (both from DAKO Corp., Carpinteria, Calif.). Incubation with the anti-human IL11Rα antibody C20 (1:15 for 45 minutes; Santa Cruz Biotechnology, Santa Cruz, Calif.) and the LSAB+ kit (DAKO) followed. Endothelial cells were immunostained by JC/70A monoclonal antibody (anti-CD31, DAKO). Positive cases were defined by the presence of cytoplasmic staining, as seen in the positive controls (paraffin sections from a pellet of HeLa cells; ATCC, Manassas, Va.) (Blanc et al., 2000). Categories 1+ to 3+ (intensity of staining in the luminal cells) were used for evaluation of benign prostatic tissues and comparison to PIN and primary prostate cancer; a scoring system based on combined intensity and percentage of immunostained tumor cells (from 0 to 300) was used to evaluate differences among specimens (Luo et al., 2002). All statistical analyses were done with S-PLUS 2000 (Math Soft Inc., Seattle, Wash.).

Phage Overlay Assays. Representative cases from the previous panel were selected, including: primary androgen-dependent tumors of various Gleason scores and pathological stages (n=10), primary androgen-independent (n=5), and prostate cancer lymph node (n=5) and bone metastases (n=6). Phage was immunolocalized as described (Arap et al., 2002). To confirm specificity for the CGRRAGGSC (SEQ ID NO:1) sequence, phage-staining inhibition was tested by co-incubation with the soluble CGRRAGGSC (SEQ ID NO:1)-GG-_{D}(KLAKLAK)₂ (SEQ ID NO:11) peptide.

Phage Internalization Assays and Immunocytochemistry. 5×10⁴ LNCaP or MDA-PCa-2b cells (ATCC) were incubated with 5×10⁹ transducing units (TU) IL-11-mimic phage in a chamber slide (Lab-Tek II, Nalge Nunc International, Naperville, Ill.). Rabbit anti-fd bacteriophage antibody (Sigma, St. Louis, Mo.) and Cy3-conjugated anti-rabbit antibody (Jackson, West Grove, Pa.) were used for phage immunodetection. Insertless fd phage was used as negative control for internalization. Cell expression of IL-11Rα was evaluated with a rabbit antibody (C20; Santa Cruz Biotechnology) that cross-reacts with both human and mouse receptors.

In vitro Protein Binding Assays. CGRRAGGSC (SEQ ID NO:1)-displaying phage (IL-11-mimic) binding to recombinant mouse IL-11Rα (R&D Systems, Minneapolis, Minn.) was assessed as described (3). Scramble phage clones displaying the peptides CRGSGAGRC (SEQ ID NO:2) or CSGGGRARC (SEQ ID NO:3), phage clones displaying the unrelated peptides CKGGRAKDC (SEQ ID NO:4) or CGSPGWVRC (SEQ ID NO:5), and insertless phage (fd-tet) were used as controls.

Induction and Quantification of Apoptosis with CGRRAGGSC (SEQ ID NO:1)—GG-_{D}(KLAKLAK)₂ (SEQ ID NO:11) Synthetic Peptide. Soluble CGRRAGGSC (SEQ ID NO:1)—GG-_{D}(KLAKLAK)₂ (SEQ ID NO:11), CGRRAGGSC (SEQ ID NO:1)-, and _{D}(KLAKLAK)₂ (SEQ ID NO:11) peptides, and the unrelated control peptide CKGGRAKDC (SEQ ID NO:4)-GG-_{D}(KLAKLAK)₂ (SEQ ID NO:11), were synthesized to our specifications at greater than 90% purity by AnaSpec (San Jose, Calif.). The unrelated control peptide CGSPGWVRC (SEQ ID NO:5)-GG-_{D}(KLAKLAK)₂ (SEQ ID NO:11) was synthesized by Genemed Synthesis, Inc. (South San Francisco, Calif.). LNCaP, MDA-PCa-2b cells (each at 3×10⁴ per well), and EF43.fgf-4 cells (7) at 2×10⁴ per well were seeded in triplicates and incubated in 96-well plates (Becton Dickinson, Franklin Lakes, N.J.) for 24-72 hours at 37° C., with serially increasing concentrations (10-100 μM) of CGRRAGGSC (SEQ ID NO:1))—GG-_{D}(KLAKLAK)₂ (SEQ ID NO:11) peptide, CGRRAGGSC (SEQ ID NO:1)-peptide alone, _{D}(KLAKLAK)₂ (SEQ ID NO:11) peptide alone, or an equimolar mixture of the unconjugated peptides CGRRAGGSC (SEQ ID NO:1)- and _{D}(KLAKLAK)₂ (SEQ ID NO:11). LNCaP cells were also exposed in parallel to increasing concentrations (20-100 μM) of CGRRAGGSC (SEQ ID NO:1)—GG-_{D}(KLAKLAK)₂ (SEQ ID NO:11) and unrelated control peptides CKGGRAKDC (SEQ ID NO:4)-GG-_{D}(KLAKLAK)₂ (SEQ ID NO:11) or CGSPGWVRC (SEQ ID NO:5)-GG-_{D}(KLAKLAK)₂ (SEQ ID NO:11), under the same conditions. Specificity of binding to IL-11Rα was additionally tested by incubating LNCaP cells with either IL-11Rα antibody (50 μg/mL; Santa Cruz Biotechnology) or rabbit IgG (Zymed Labs., San Francisco, Calif.) for 1 hour, and then by adding 40 μM CGRRAGGSC (SEQ ID NO:1)—GG-_{D}(KLAKLAK)₂ (SEQ ID NO:11) peptide for 3 hours. Response was evaluated by a cell viability assay (WST-1; Roche, Mannheim, Germany).

Cloning of Fd-based phage with specific inserts. DNA sequences encoding the GRP78 aptamers were cloned into SfiI-digested fUSE5 vector. Briefly, 500 ng of the corresponding synthetic oligonucleotides (Genemed Synthesis Inc., San Francisco, Calif.) were converted to double-stranded DNA by PCR amplification using the primers 5′-GTGAGCCGGCTGCCC-3′ (SEQ ID NO:13) and 5′-TTCGGCCCCAGCGGC-3′(SEQ ID NO:14) (Sigma Genosys, The Woodlands, Tex.) and 2.5 U of Taq-DNA polymerase (Promega, Madison, Wis.) in 20 μl as follows: 94° C. for 2 minutes, followed by 35 cycles at 94° C. for 30 seconds, 60° C. for 30 seconds, and 72° C. for 30 seconds, followed by 72° C. for 5 minutes. The double-stranded DNA generated contained BglI restriction sites in the insert-flanking region. They were purified by using a QIAquick nucleotide removal kit (QIAGEN, Gmbh, Hilden, Germany) and eluted from each QIAquick column (QIAGEN) by 50 μl washes with dH₂O. The oligonucleotides were digested with BglI for 2 hours at 37° C., re-purified and ligated into SfiI-digested fUSE5 vector. Finally, the plasmids were electroporated into MC1061 Escherichia coli. DNA from each of the phage clones produced was PCR amplified and sequenced to verify the correct insertion.

In vitro phage binding assays. GRP78, HSP70, HSP90 (all from Stressgen, Victoria, Canada) and bovine serum albumin (BSA) were immobilized on microtiter wells of 96-well plates overnight at 4° C. Wells were washed twice with phosphate-buffered saline (PBS), blocked with PBS containing 3% BSA for 1 hour at room temperature (RT), and incubated with 10⁹ transducing units (TU) of WIFPWIQL (SEQ ID NO:6)-phage, WDLAWMFRLPVG (SEQ ID NO:7)-phage, or insertless control phage (Fd-tet) in 50 μl of PBS containing 1.5% BSA. After 2 hours at RT, wells were washed with PBS, and bound phage were recovered by infection with host bacteria (log-phase Escherichia coli K91 kan; OD₆₀₀≈2). Aliquots of the bacterial culture were plated onto Luria-Bertani broth (LB) agar plates supplemented with 40 μg/ml tetracycline and 100 μg/ml kanamycin. Plates were incubated overnight at 37° C., and phage TU were counted in triplicate plates. Increasing concentrations of synthetic peptides WIFPWIQL (SEQ ID NO:6), WDLAWMFRLPVG (SEQ ID NO:7)-, and an unrelated control peptide CARAC (SEQ ID NO:9) (Genemed Synthesis Inc., San Francisco, Calif.) were used to evaluate competitive inhibition of phage binding. All peptides were solubilized in a standard stock solution of 10% dimethylsulfoxide (DMSO) and diluted to working concentrations for the assays.

Cell-binding assays. Biopanning and Rapid Analysis of Selective Interactive Ligands (BRASIL) method was used to evaluate phage binding to intact cells. In brief, cultured human prostate cancer-derived DU 145 cells were detached with ethylenediaminetetraacetate (EDTA) and resuspended in Dulbecco's modified Eagle's medium (DMEM) containing 1% BSA at 4×10⁶ cells per ml. The cell suspension (50 μl) was incubated with 10⁹ of WIFPWIQL (SEQ ID NO:6)—phage, WDLAWMFRLPVG (SEQ ID NO:7)—phage, or insertless Fd-tet phage (negative control) at 4° C. in constant slow rotation. After 2 hours, the phage/cell mixture (aqueous phase) was gently transferred to the top of a non-miscible organic phase (200 μl solution in a 400 μl Eppendorf tube) consisting of dibutyl phtalate:cyclohexane (9:1 [v:v]; d=1.03 g ml⁻¹) and centrifuged at 10,000 g for 10 minutes at 4° C. The tube was then snap frozen in liquid nitrogen, the bottom of the tube was sliced off, and the cell-phage pellet was isolated. Cell membrane bound phage were recovered by infection with the host bacteria. A polyclonal rabbit anti-GRP78 antibody (Stressgen, Victoria, Canada), and an unrelated control antibody at the same dilution; recombinant GRP78 (Stressgen, Victoria, Canada), unrelated control proteins and synthetic cognate or control peptides (each at 100 μg/ml) were used to evaluate competitive inhibition of phage binding.

Establishment of mice bearing human tumor xenografts. Male athymic nu/nu (nude) mice were obtained commercially from Harlan-Sprague-Dawley (Indianapolis, Ind.). Human prostate cancer xenografts were established by injection of DU 145 cells (10⁶ cells in a 200 μl DMEM) in the subcutaneous tissue of 2 months old male nude mice.

Tumor targeting in vivo. In vivo targeting experiments with phage were performed as described. Briefly, Avertin anesthetized athymic nude mice bearing size-matched human DU 145 xenografts were injected intravenously (tail vein) with 10¹⁰ TU of the WIFPWIQL (SEQ ID NO:6)—phage, WDLAWMFRLPVG (SEQ ID NO:7)—phage, RGD-4C phage (positive control), or Fd-tet phage (negative control) in DMEM. Three mice were injected with each phage. After 24 hours, tumor-bearing mice were perfused through the heart with 20 ml of 4% paraformaldehyde. Tumor and control organs (brain and spleen) were dissected from each mouse and fixed in 4% PFA/PBS solution for 24 hours. Organs were paraffin-embedded and sectioned into 5 μm specimens for specific phage staining.

Immunohistochemical phage staining of mice organs. Immunohistochemistry on sections of fixed mouse paraffin-embedded tissues was done with the LSAB+ peroxidase kit (DAKO, Carpinteria, Calif.). Briefly, slides were deparaffinized and rehydrated with xylene and graded alcohols, blocked for endogenous peroxidases, and antigen-retrieved in a microwave oven by treatment with an antigen retrieval solution (DAKO). Slides were blocked for non-specific protein binding, and a rabbit anti-bacteriophage primary antibody (Sigma) was added (150 μl at 1:500 dilution). After 1 hour, slides were washed 3 times with 0.1% Tween 20 in Tris buffered saline (TBST; LSAB+ peroxidase kit), and the peroxidase-conjugated anti-rabbit secondary antibody was added. The slides were washed again 3 times with TBST and developed with the substrate-chromogen 3,3′-Diaminobenzidine (DAB; DAKO). Counterstain was achieved by a 20 seconds immersion in 100% hematoxylin, and the slides were dehydrated (with graded alcohols and xylene) and mounted. All sections and controls from each specimen were included in the same staining run to minimize experimental variability.

Phage binding assays on human prostate cancer. Immunohistochemistry on sections of fixed human paraffin-embedded bone metastases was performed with LSAB+ peroxidase kit (DAKO). Briefly, surgical specimens of prostate cancer patients diagnosed with bone metastases were obtained from the University of São Paulo Medical School, after approval from their Institutional Review Board. Sections (5 μm) were deparaffinized and rehydrated, blocked for endogenous peroxidases and for non-specific protein binding. An anti-GRP78 goat polyclonal antibody (C-20, sc-1051; Santa Cruz Biotechnology, Santa Cruz, Calif.) and an unrelated control goat polyclonal isotype antibody (goat IgG-reagent grade; Sigma, St. Louis Mo.) at the same immunoglobulin concentration were used to evaluate competitive inhibition of phage binding. Tissues sections were incubated with media alone, with the anti-GRP78 antibody or with the control antibody at the same immunoglobulin concentration for 1 hour at RT. Next, 2×10⁹ TU of WIFPWIQL (SEQ ID NO:6)—phage and WDLAWMFRLPVG (SEQ ID NO:7)—phage were incubated for 2 hours at RT. An anti-bacteriophage antibody (Sigma) was added to the slides (150 μl volume of a 1:500 dilution) and incubated for 1 hour at RT. After 3 washes with TBST, the peroxidase-conjugated anti-rabbit secondary antibody was added. Slides were washed 3 times with TBST and developed with the DAB. Slides were counterstained by a 20 seconds immersion in 100% hematoxylin, dehydrated, and mounted.

Next, whether the phage would block anti-GRP78 antibody staining was tested. Briefly, after deparaffinization, rehydration and protein and peroxidase blockages, 2×10⁹ TU of WIFPWIQL (SEQ ID NO:6)—phage, WDLAWMFRLPVG (SEQ ID NO:7)—phage, fd-tet (negative control) or media alone were added to the slides and incubated for 2 hours. Next the anti-GRP78 or the control antibody at an equivalent immunoglobulin concentration were added to the slides and incubated for 1 hour at RT. Slides were washed three times with TBST and the peroxidase-conjugated secondary antibody was added. After 3 washes with TBST, development was achieved with the DAB substrate. Slides were counterstained by a 20 seconds immersion in 100% hematoxylin, dehydrated, and mounted.

Cell apoptosis assays. Peptides were synthesized to our specifications at greater than 95% purity (Genemed Synthesis Inc., San Francisco, Calif.). Apoptosis was induced with a pro-apoptotic motif _{D}(KLAKLAK)₂ that disrupts mitochondrial membranes and is inert to eukaryotic plasma membranes. An equimolar concentration of the targeted [(WIFPWIQL (SEQ ID NO:6)-GG-_{D}(KLAKLAK)₂ (SEQ ID NO:11) and WDLAWMFRLPVG (SEQ ID NO:7)-GG-_{D}(KLAKLAK)₂) (SEQ ID NO:11)] and untargeted [(WIFPWIQL (SEQ ID NO:6)-+(KLAKLAK)₂ (SEQ ID NO:11) and WDLAWMFRLPVG (SEQ ID NO:7)-+(KLAKLAK)₂ (SEQ ID NO:11)] peptides was used. Human prostate cancer-derived DU 145 cells were grown in tissue chamber slides (Lab-Tek II Chamber Slide System; Nalge Nunc International Corp., Naperville, Ill.). Cells were washed with PBS and incubated with 30 μM (in 300 μl DMEM supplemented with 10% FBS, penicillin and streptomycin) of the targeted and untargeted peptides for 6 hours. Pure DMEM was used as an internal negative control. Apoptosis was detected with the Annexin-V-FLUOS Staining kit (Roche, Manheim, Germany) according to the manufacturer's instructions.

Targeted treatment of nude mice bearing human prostate carcinoma xenografts. DU145-derived tumor xenografts were established in male nude mice as described above. GRP78-targeting peptides used for therapy were coupled to the pro-apoptotic motif _{D}(KLAKLAK)₂ (SEQ ID NO:11) and controls were treated with an equimolar concentration of the GRP78-targeting peptides and the pro-apoptotic motif. Mice were divided into groups of seven animals and treatment started when mean tumor volume for each group was around 200 mm³. Two-dimensional measurements of tumors were made by caliper on anesthetized mice, and were used to calculate tumor volume. The mice were anesthetized with Avertin and the peptides were administered at a dose of 300 μg/week per mouse, given slowly through the tail vein in 200 μl of DMEM.

Statistical analysis. Experiments are expressed as mean ±standard errors of the means (SEM) of triplicate plates and analyzed by using the two-tailed Student's t test (t test). Tumor volumes were calculated individually for each mouse throughout the study and results are also expressed as mean ±SEM for all the groups.

B. Results

1. Targeting Tumor Cells Using Differential Expression of IL-11Rα

To begin to evaluate IL-11Rα in the context of human prostate cancer, morphologic (immunohistochemistry) and functional (targeting and internalization) assays were used. First, the expression of IL-11Rα in a large panel of androgen-dependent and androgen-independent prostate cancer specimens (n=99) by using both a specific antibody and an IL-11-mimic ligand phage clone (displaying the peptide CGRRAGGSC (SEQ ID NO:1)-was used. Moreover, the targeting of the IL-11-mimic peptide in human prostate cancer-derived cells was tested. Finally, the internalization capability of the IL-11Rα by measuring uptake of IL-11-mimic phage and programmed cell death induction in vitro mediated by a targeted pro-apoptotic synthetic peptide was assessed.

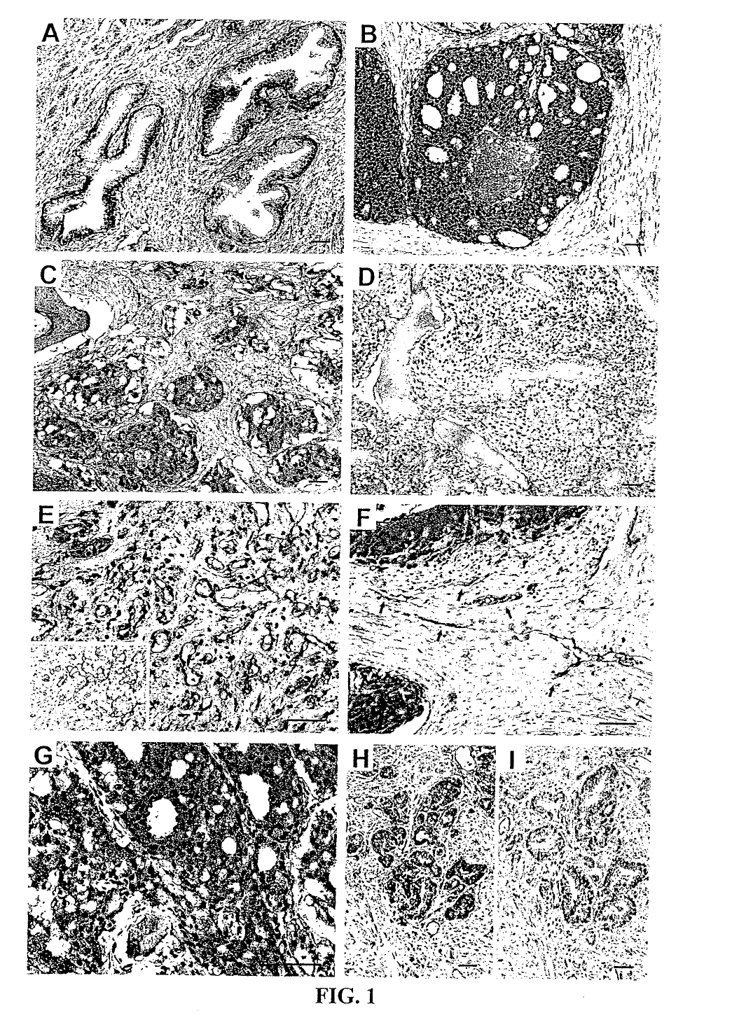

The immunohistochemical expression of IL-11Rα in formalin-fixed paraffin-embedded tissue samples including the entire spectrum of prostate cancer from pre-malignant PIN to androgen-independent metastatic tumors, and normal prostate from the peripheral, transition, and central zones was tested (Table 3). As examined with an anti-IL-11Rα antibody (FIGS. 1A, 1B, 1C and 1D), expression in normal prostatic glands from the different zones was low, typically localized in the basal cell compartment with or without staining of the luminal cells. Expression of the receptor in PIN and primary androgen-dependent prostate cancer samples was significantly higher than in their benign counterparts from the same areas (P<0.0001 for both comparisons, Wilcoxon signed rank test). The extent and intensity of staining were heterogeneous among and within androgen-dependent tumor samples, but clearly increased in association with rising Gleason score and tumor stage (Table 3). In contrast, primary androgen-independent cancer showed a more homogeneous pattern of staining, with more than 80% cells displaying moderate/strong intensity in 8 of 10 (80%) samples. Expression in lymph node metastases (n=12) was also intense in most of the tumor cells regardless of their androgen-sensitivity status or anatomical origin. Similarly, prostate cancer cells metastatic to the bone marrow displayed a homogeneous moderate to strong intensity of staining in 5 of 6 (83%) specimens (all androgen-independent). Moreover, some small-caliber blood vessels in androgen-independent primary and metastatic tumors showed striking IL-11Rα immunoreactivity in 17 of 24 (71%) samples—confirmed by CD31 (PECAM-1) staining on serial sections—as opposed to a less consistent pattern in benign tissues and androgen-dependent tumors analyzed (FIG. 1E).

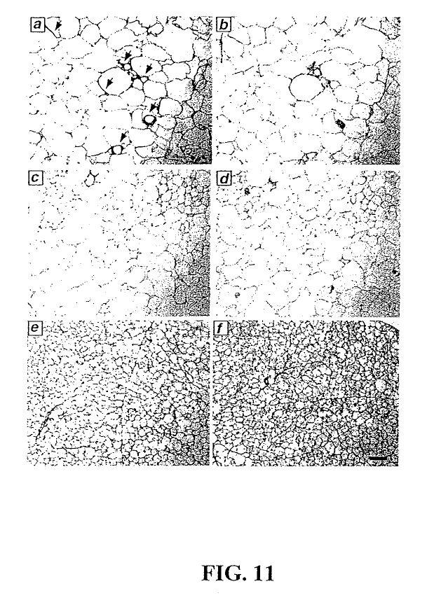

FIG. 1. IL-11Rα expression in normal prostate and primary and metastatic prostate cancer. FIG. 1A, Normal glands from the peripheral zone showing predominant staining in the basal cell compartment and area of transitional metaplasia (arrow), and no staining in the luminal cell layers. FIG. 1B, strong (3+) positive staining in high-grade primary androgen-dependent prostatic adenocarcinoma. FIG. 1C, homogeneous (3+) expression in prostate cancer metastatic to bone. FIG. 1D, negative control (normal Ig). FIG. 1E, positive staining in small blood vessels around malignant tumor tissue in bone matrix, confirmed by CD31 immunostaining on serial tissue sections (see inset for a representative section). FIGS. 1F and 1G, IL-11-mimic phage overlays. FIG. 1F, high-grade, androgen-independent primary tumor showing strong (3+) and homogeneous staining in malignant epithelium and associated vessels (arrows). FIG. 1G, strong homogeneous expression in prostate cancer metastatic to bone. FIGS. 1H and 1I, IL-11-mimic phage-staining inhibition. Phage localization to primary prostate cancer glands (FIG. 1H) was abolished (serial tissue sections) by co-incubation with soluble CGRRAGGSC (SEQ ID NO:1)—GG-_{D}(KLAKLAK)₂ (SEQ ID NO:11) peptide (FIG. 11). Bar, 50 μm in all panels.

TABLE 3
Interleukin-11 receptor α expression in prostate cancer

| | | | |
|---|---|---|---|
| Specimen | n | Median score (range) | p-value |

Normal prostate

| | | | | |
|---|---|---|---|---|
| Peripheral zone | 62 | 1+ | (1-2) | NS |
| Transition zone | 51 | 1+ | (1-2) | |
| Central zone | 40 | 1+ | (1-2) | |

Benign conditions

| | | | | |
|---|---|---|---|---|
| Benign prostatic hyperplasia | 15 | 1+ | (1-2) | — |
| Stromal nodule | 2 | 1+ | (1-2) | — |
| Atrophy | 10 | 2+ | (1-2) | — |
| Transitional metaplasia | 18 | 2+ | (1-2) | — |
| PIN, high grade | 23 | 2+ | (1-3) | — |
| Primary prostate cancer | | | | |

| | | | |
|---|---|---|---|
| Androgen-dependent | 71 | 2+ (1-3)/180 (50-290) | — |

Zonal origin

| | | | | |
|---|---|---|---|---|
| Peripheral zone | 55 | 190 | (50-290) | 0.0003* |
| Transition zone | 16 | 135 | (50-250) | |

Gleason score

| | | | | |
|---|---|---|---|---|
| ≦7 (3 + 4) | 26 | 150 | (50-260) | 0.004* |
| ≧7 (4 + 3) | 38 | 200 | (100-290) | |

Pathological stage

| | | | | |
|---|---|---|---|---|
| pT₂-pT₃ₐ | 42 | 175 | (50-290) | 0.046* |
| pT_{3b}-pT_{any}pN₁ | 22 | 210 | (100-280) | |

Serum PSA (ng/mL)†

| | | | | |
|---|---|---|---|---|
| <10 | 48 | 180 | (50-280) | NS |
| ≧10 | 14 | 200 | (100-290) | |
| Androgen-independent | 10 | 250 | (80-300) | — |
| Metastatic prostate cancer | | | | |

Lymph nodes

| | | | | |
|---|---|---|---|---|
| Androgen-dependent | 4 | 235 | (200-290) | NS |
| Androgen-independent | 8 | 235 | (190-300) | |
| Bone marrow | 6 | 270 | (140-290) | — |

†Serum PSA not available in 2 of 64 samples.
*Mann-Whitney rank sum test.
NS, non-significant.

To establish if similar differences in expression were also apparent and detectable for the epitope recognized by the IL-11-mimic phage, phage overlay assays were performed on representative cases from the previous panel (n=26) including primary androgen-dependent and independent tumors and prostate cancer metastases (FIGS. 1F and 1G). The pattern of phage-bound staining matched that of the antibody, confirming that the IL-11 mimic phage co-localizes with the IL-11Rα receptor in tissue sections. Specificity was further confirmed when the staining was inhibited by co-incubation with the CGRRAGGSC (SEQ ID NO:1)—GG-_{D}(KLAKLAK)₂ (SEQ ID NO:11) peptide (FIGS. 1H and 1I). Differential expression of normal vs. tumor tissues appeared more evident than in cases with previous anti-IL-11Rα antibody low to moderate expression. In general agreement with previous findings, most endothelia in these samples were recognized by the IL-11-mimic phage.

To model the functionality of the targeting system in vitro, the human prostate cancer-derived cell lines MDA-PCa-2b and LNCaP were chosen because of their androgen-sensitive and PSA-expressing features and also because such cells express IL-11Rα; as a negative control, the mouse mammary tumor-derived cells EF43.fgf-4 were selected because expression of IL-11Rα was not detectable (data not shown). By using this panel of cells, the targeting of the IL-11Rα and internalization of a synthetic peptide consisting of an IL-11-mimic domain linked to a well-established pro-apoptotic domain, _{D}(KLAKLAK)₂ (SEQ ID NO:11) was evaluated _{D}(KLAKLAK)₂ (SEQ ID NO:11) is an amphipathic, α-helix-forming anti-microbial peptide that preferentially disrupts eukaryotic mitochondrial membranes rather than plasma membranes when internalized by a ligand-receptor system.

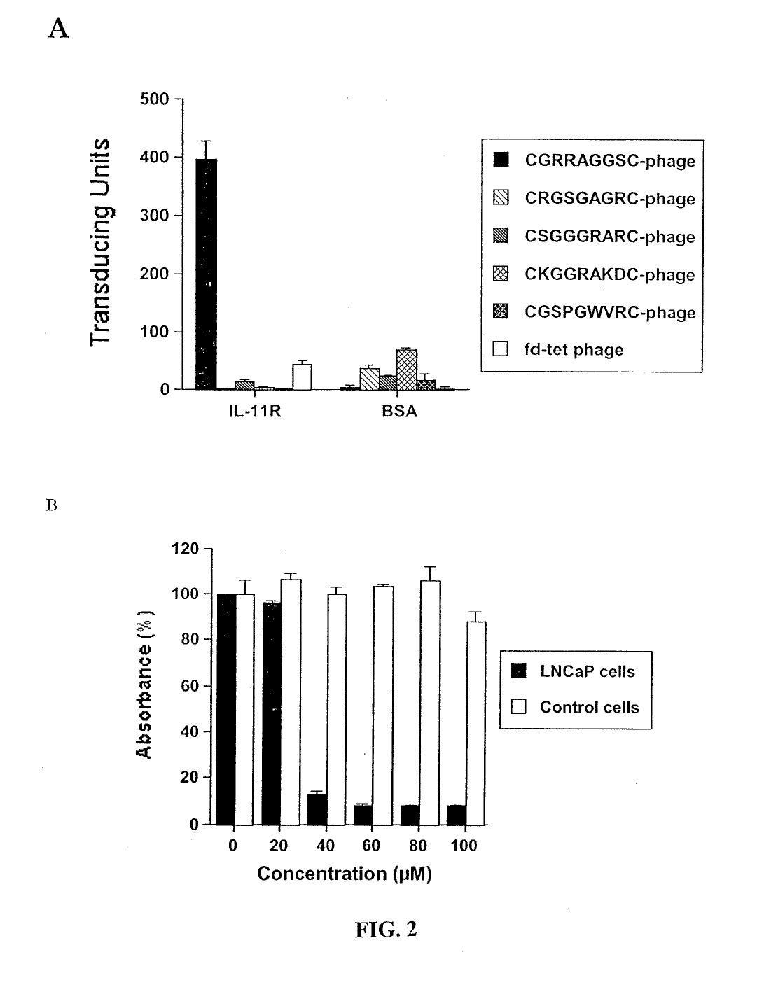

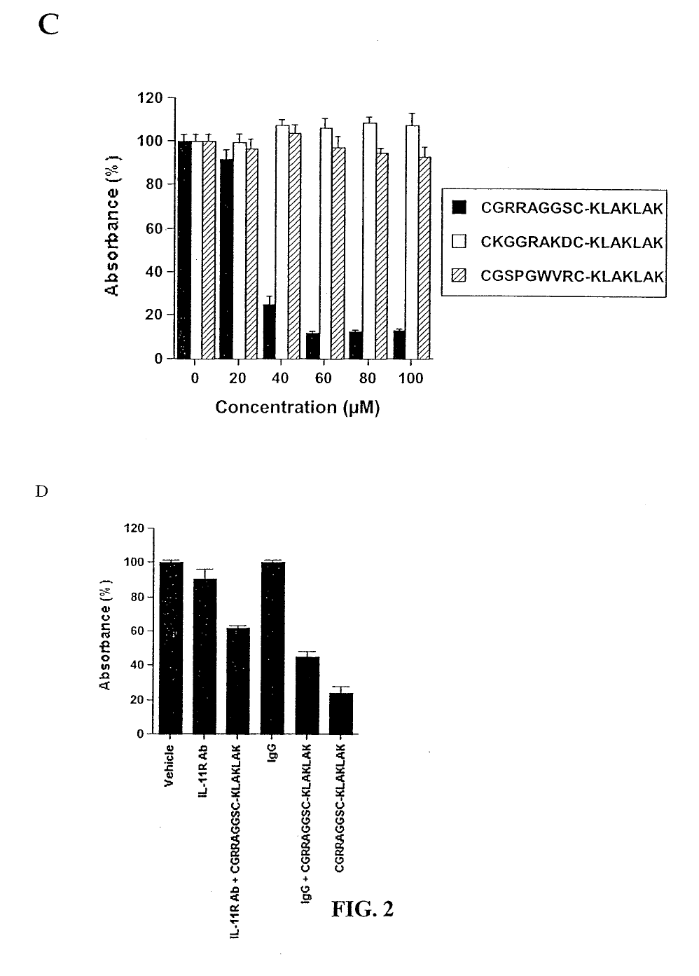

The in vitro binding of CGRRAGGSC (SEQ ID NO:1)—displaying phage was evaluated and several control phage for IL-11Rα (FIG. 2A). Binding of CGRRAGGSC (SEQ ID NO:1)-displaying phage was significantly higher than that of control phage, including: phage displaying scrambled IL-11-mimic peptides (CRGSGAGRC (SEQ ID NO:2) or CSGGGRARC (SEQ ID NO:3), unrelated peptide sequences (CKGGRAKDC (SEQ ID NO:4) or CGSPGWVRC (SEQ ID NO:5), and insertless phage (fd-tet) (P<0.0001 for each case, t-test).

FIGS. 2A, 2B, 2C and 2D. CGRRAGGSC (SEQ ID NO:1)—GG-_{D}(KLAKLAK)₂ (SEQ ID NO:11) binds specifically to IL-11Rα and induces apoptosis in IL-11Rα-positive prostate cancer cell lines. FIG. 2A, in vitro binding to immobilized IL-11Rα of CGRRAGGSC (SEQ ID NO:1)-displaying or control phage, including: scrambled peptides (CRGSGAGRC (SEQ ID NO:2) or CSGGGRARC (SEQ ID NO:3), unrelated peptide sequences (CKGGRAKDC (SEQ ID NO:4) or CGSPGWVRC (SEQ ID NO:5), and insertless phage (fd-tet). FIG. 2B, dose-response effect of CGRRAGGSC (SEQ ID NO:1)-GG-_{D}(KLAKLAK)₂ (SEQ ID NO:11) No on IL-11Rα-expressing LNCaP cells and lack of effect on IL-11Rα-deficient EF43fgf-4 cells. Both cell lines were treated with increasing concentrations of CGRRAGGSC (SEQ ID NO:1)-GG-_{D}(KLAKLAK)₂ (SEQ ID NO:11) for 24 hours. FIG. 2C, cell killing selectivity of CGRRAGGSC (SEQ ID NO:1)-GG-_{D}(KLAKLAK)₂ (SEQ ID NO:11) vs. control peptides. LNCaP cells were independently incubated for 72 hours with increasing concentrations of CGRRAGGSC (SEQ ID NO:1)-GG-_{D}(KLAKLAK)₂ (SEQ ID NO:11) peptide (CGRRAGGSC (SEQ ID NO:1)-KLAKLAK (SEQ ID NO:11)) or the unrelated peptides CKGGRAKDC (SEQ ID NO:4)-GG-_{D}(KLAKLAK)₂ (SEQ ID NO:11) (CKGGRAKDC (SEQ ID NO:4)-KLAKLAK₂ (SEQ ID NO:11)) or CGSPGWVRC (SEQ ID NO:5)-GG-_{D}(KLAKLAK)₂ (SEQ ID NO:11) (CGSPGWVRC (SEQ ID NO:5)-KLAKLAK₂ (SEQ ID NO:11)). FIG. 2D, IL-11Rα antibody-mediated inhibition of pro-apoptotic effect of CGRRAGGSC (SEQ ID NO:1)-GG-_{D}(KLAKLAK)₂ (SEQ ID NO:11). LNCaP cells were incubated for 4 hours with anti-IL-11Rα antibody (IL-11R Ab), anti-IL-11Rα antibody plus CGRRAGGSC (SEQ ID NO:1)-GG-_{D}(KLAKLAK)₂ (SEQ ID NO:11) peptide, non-specific IgG, non-specific IgG plus CGRRAGGSC (SEQ ID NO:1)-GG-_{D}(KLAKLAK)₂ (SEQ ID NO:11) peptide, or CGRRAGGSC (SEQ ID NO:1)-GG-_{D}(KLAKLAK)₂ (SEQ ID NO:11) peptide alone. Drug response was assessed by the WST-1 cell viability assay. Absorbance obtained for cells incubated with vehicle alone was set to 100% in graphs FIGS. 2B, 2C and 2D. Bars, mean ±standard error of the mean in all graphs.

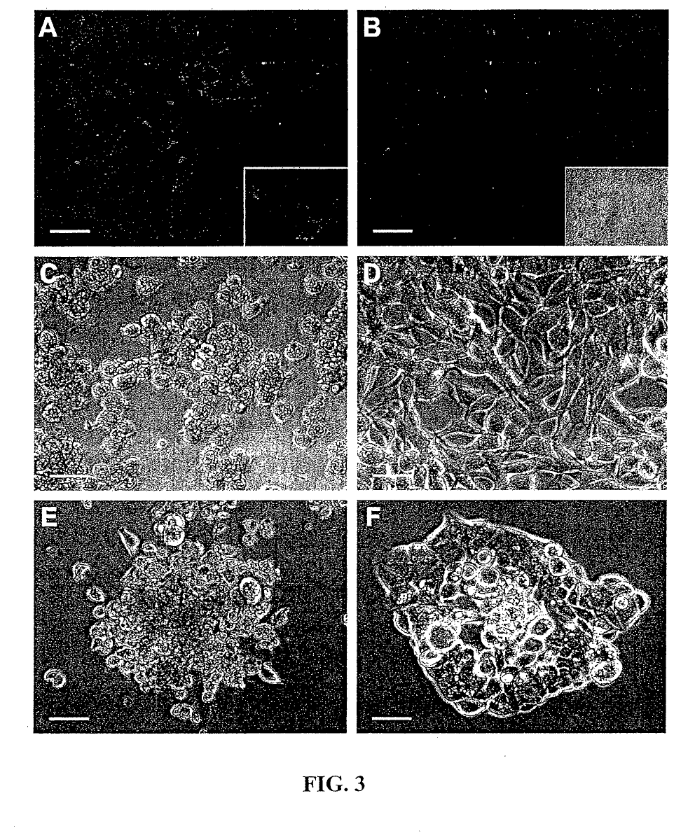

Immunofluorescence peptide-mediated IL-11-mimic phage internalization in LNCaP (FIGS. 3A and 3B) and MDA-PCa-2b cells (not shown) was demonstrated. The chimeric synthetic peptide CGRRAGGSC (SEQ ID NO:1)-GG-_{D}(KLAKLAK)₂ (SEQ ID NO:11) induced dose-dependent programmed cell death in the prostate cancer cells tested. In contrast, no significant effect was observed on the IL-11Rα-deficient EF43.fgf-4 cells within the same dose range (FIG. 2B). In experiments performed under similar conditions, incubation of LNCaP and MDA-PCa-2b cells with control peptides CGRRAGGSC (SEQ ID NO:1), _{D}(KLAKLAK)₂ (SEQ ID NO:11), an equimolar mixture of uncoupled CGRRAGGSC (SEQ ID NO:1) and _{D}(KLAKLAK)₂ (SEQ ID NO:11) (FIGS. 3C, 3D, 3E and 3F), or unrelated peptides CKGGRAKDC-GG-_{D}(KLAKLAK)₂ SEQ ID No. or CGSPGWVRC (SEQ ID NO:5)-GG-_{D}(KLAKLAK)₂ (SEQ ID NO:11). (FIG. 2C), showed no measurable toxic effects. The pro-apoptotic effect of CGRRAGGSC (SEQ ID NO:1)-GG-_{D}(KLAKLAK)₂ (SEQ ID NO:11) on LNCaP cells was also significantly inhibited by co-incubation with an anti-IL-11Rα antibody, both when compared with CGRRAGGSC (SEQ ID NO:1)-GG-_{D}(KLAKLAK)₂ (SEQ ID NO:11). alone (P=0.008, t-test) or non-specific IgG (P=0.02, t-test; FIG. 2D).

FIG. 3A. IL-11-mimic phage internalization and induction of programmed cell death with CGRRAGGSC (SEQ ID NO:1)-GG-_{D}(KLAKLAK)₂ (SEQ ID NO:11) synthetic peptide. FIG. 3A, IL-11-mimic phage internalization on LNCaP cells. Note distribution in cell projections and around the nucleus (inset). FIG. 3B, insertless fd phage was used as negative control for internalization (phase-contrast in inset). FIGS. 3C, 3D, 3E and 3F, induction of programmed cell death with CGRRAGGSC (SEQ ID NO:1)-GG-_{D}(KLAKLAK)₂ (SEQ ID NO:11) synthetic peptide. LNCaP (FIGS. 3C and 3D) or MDA-PCa-2b (FIGS. 3E and 3F) cells were incubated with 50 μM CGRRAGGSC (SEQ ID NO:1)-GG-_{D}(KLAKLAK)₂ (SEQ ID NO:11) (FIGS. 3C and 3E) or an equimolar mixture of unconjugated CGRRAGGSC (SEQ ID NO:1)- and _{D}(KLAKLAK)₂ (SEQ ID NO:11) (FIGS. 3D and 3F). Morphologic evidence of programmed cell death is observed after treatment with the targeted pro-apoptotic peptide. Bar, 50 μm in all panels.

Together, these histological and functional findings establish the presence of a high and homogeneous IL-11Rα expression in primary androgen-independent and metastatic prostate cancer, and blood vessels in the majority of these specimens. On an expanded set of clinically annotated samples, up-regulation of IL-11Rα expression in primary androgen-dependent prostate cancer was demonstrated. These data indicate a gradual increase in epithelial expression of IL-11Rα that directly correlates with the clinical and pathological progression of prostate cancer. A potential function for the ligand-receptor system IL-11:IL-11α was demonstrated. Consistently, by using unrelated technology, a role for the IL-11 molecular pathway in the progression of malignant human tumors metastatic to bone has recently been proposed (Kang, et. al. 2003), possibly related to the activation of STAT3 downstream from the IL-11Rα (Campbell et al. 2001). Prospective studies on the pathogenic or prognostic value for this receptor in prostate cancer are ongoing.

In summary, the high expression of the IL-11Rα in androgen-independent disease and its associated blood vessels offers an opportunity for therapeutic targeting of a tumor with no curative treatment when metastatic. The tools provided here may enable therapeutic targeting of the IL-11Rα in prostate cancer. Finally, this study provides further support for the use of direct combinatorial screenings on patients in the development of anti-cancer targeted therapies in the context of human disease.

2. Targeting Tumor Cells Using Differential Expression of GRP78

Another study used GRP78 as a potential molecular target for cancer (eg. prostate cancer). GRP78 has been identified on the cell surface of tumor cells. Here, two GRP78-targeting phage clones in vitro were validated. Then it was demonstrated that the selected phage clones specifically target prostate cancer cells in vitro and home to a human prostate cancer xenograft in a mouse model. It was also shown that the phage clones bind to human prostate cancer bone metastases. Finally, it was shown that the selected GRP78-targeting peptides, when coupled to a pro-apoptotic motif, are able to induce cell death in vitro and prevent tumor growth in vivo by 70%.

Here, whether GRP78-ligands could serve as potential targeted therapy agents for prostate cancer was evaluated. Experiments were devised to assess GRP78-based protein-protein interactions in solid phase, cell lines, tumor xenografts, and human prostate cancer tissue samples. It was shown that ligand peptides to GRP78 (i) target prostate cancer cells in vitro, (ii) home to prostate cancer-derived xenografts in vivo, (iii) bind to human prostate cancer bone metastases and, when coupled to a pro-apoptotic peptide (iv) induce programmed cell death and (v) prevent tumor growth in a human prostate cancer xenograft. Together, these data indicate that GRP78 is a molecular target in prostate cancer that can be used for targeted therapy development. It is also a likely target for other cancer cells. Therefore, in one embodiment, peptides may be used to target GRP78 for specific cancer diagnosis. In other embodiments, targeting peptides that bind GRP78 may be used to deliver agents such as pro-apoptotic agents to cancer cells to induce apoptosis.

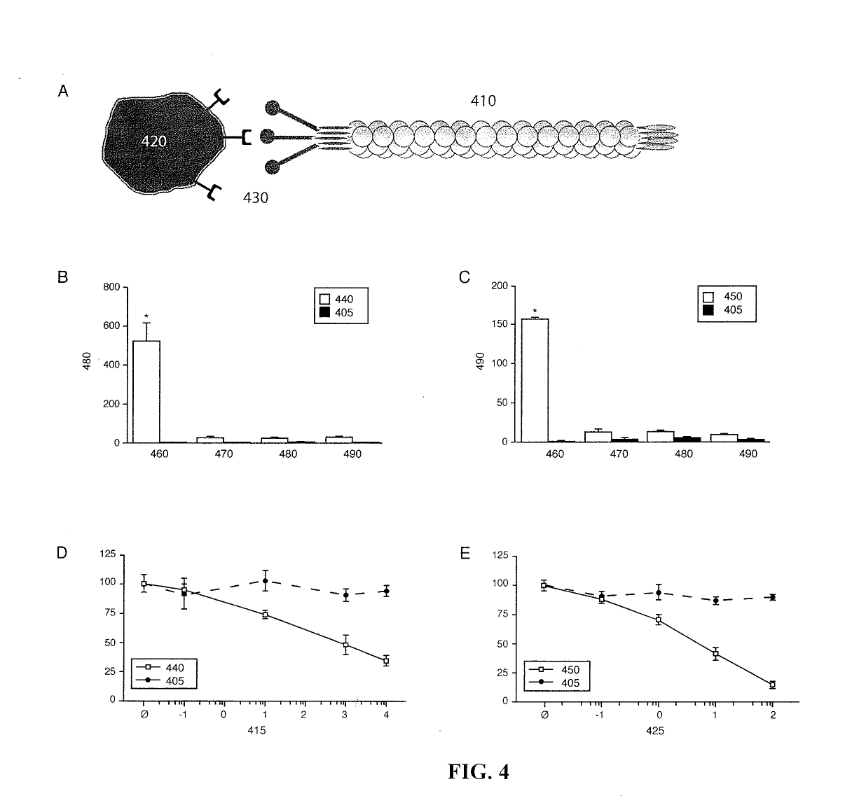

FIG. 4A is a schematic representation of phage displaying peptides binding to a target on the cell surface. This figure is an example of any ligand-receptor pair.

Peptide aptamers bind specifically to immobilized GRP78. Two phage vectors displaying the GRP78-binding peptides WIFPWIQL (SEQ ID NO:6) and WDLAWMFRLPVG (SEQ ID NO:7) as pIII recombinant fusion coat proteins. The binding and specificity of WIFPWIQL (SEQ ID NO:6)-phage 440 (FIG. 4B) and of WDLAWMFRLPVG (SEQ ID NO:7)-phage 450 (FIG. 4C) were tested to recombinant GRP78 in microtiter wells. WIFPWIQL (SEQ ID NO:6) and WDLAWMFRLPVG (SEQ ID NO:7)-phage bound significantly more to GRP78 in vitro than to control proteins including HSP70 470, HSP90 480, and BSA 490. WIFPWIQL (SEQ ID NO:6)-phage (870-fold; t-test, P<0.001) and WDLAWMFRLPVG (SEQ ID NO:7)-phage (260-fold; t-test, P<0.001) bound significantly more to immobilized GRP78 in vitro than the negative control phage (fd-tet). A dose-dependent inhibition of WIFPWIQL (SEQ ID NO:6)-phage 440 (FIG. 4D) and WDLAWMFRLPVG (SEQ ID NO:7)-phage 450 (FIG. 4E) was observed binding to GRP78 by the corresponding synthetic peptides; control peptides with unrelated sequences had no inhibitory effect. Together, these data show that selected peptide aptamers can specifically bind to GRP78.

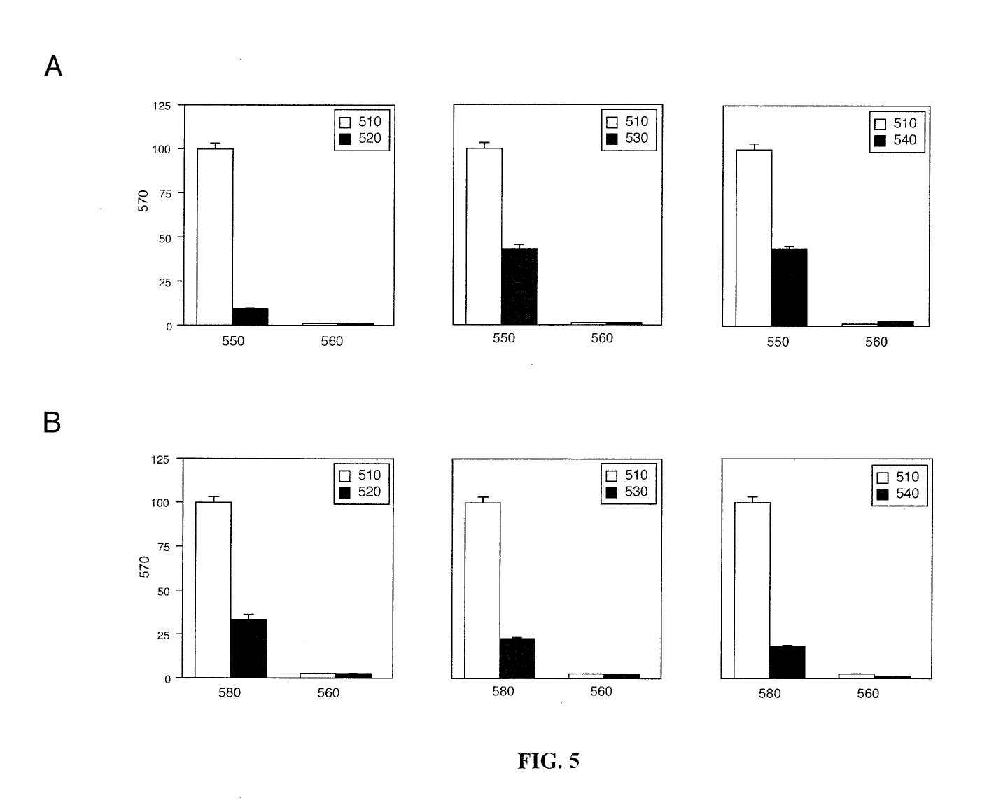

GRP78-binding phage clones bind specifically to prostate cancer cells. Having determined the binding specificity of aptamers to GRP78 in vitro, the binding of filamentous phage clones displaying WIFPWIQL (SEQ ID NO:6) (FIG. 5A) 550 and WDLAWMFRLPVG (SEQ ID NO:7) (FIG. 5B) 580 to intact DU145 human prostate cancer cells by using an aqueous-organic phase separation was evaluated. A 30-fold higher binding (t test, P<0.001) to DU145 cells was found for WIFPWIQL (SEQ ID NO:6)-phage 550 and WDLAWMFRLPVG (SEQ ID NO:7)-580 phage compared to the control phage (fd-tet) 560. The interaction of either WIFPWIQL (SEQ ID NO:6)-phage (FIG. 5A) or WDLAWMFRLPVG (SEQ ID NO:7)-phage (FIG. 5B) to DU145 cell surfaces via GRP78 was specific, as an anti-GRP78 polyclonal antibody (FIGS. 5A and 5B, left panels 520), the recombinant GRP78 (FIGS. 5A and 5B, middle panels 530), and the corresponding synthetic peptides (FIGS. 5A and 5B, right panels 540) inhibited the binding activity. Control isotypic antibodies, unrelated control proteins and peptides did not affect binding of the GRP78-binding phage.

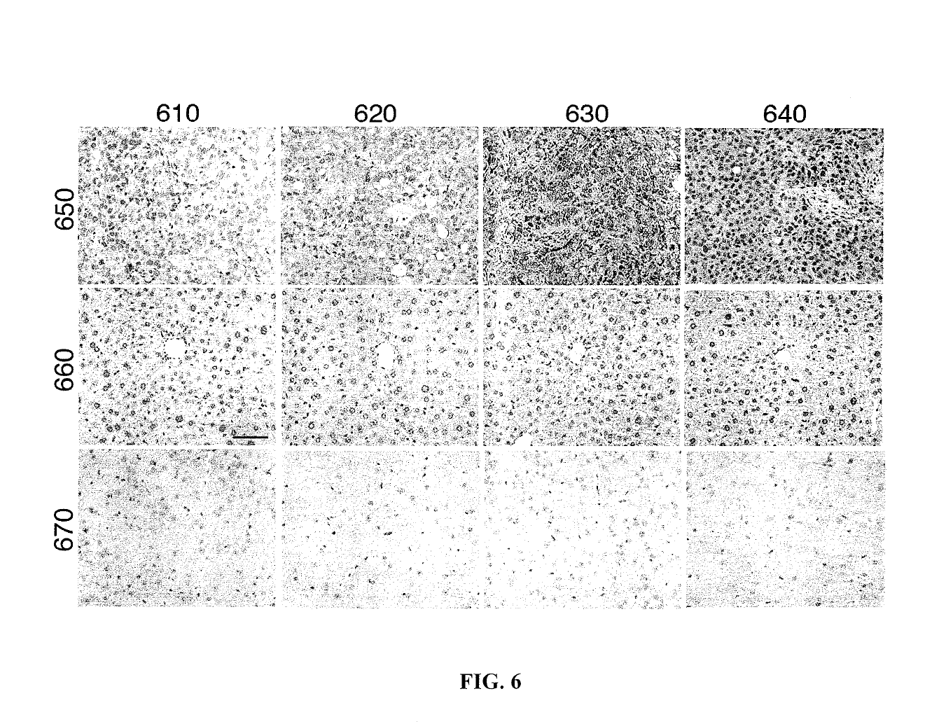

GRP78-binding Phage Homes to Prostate Cancer Xenografts In Vivo after Systemic Administration. To determine the ability of GRP78-binding phage clones to home to tumors in vivo, the selected phage 630 640 or control phage 610 620 were intravenously injected into nude mice bearing DU145-derived xenografts. After 24 hours, the mice were sacrificed and the tumors 650 and control organs 660 670 were collected and analyzed for phage staining. After 24 hours circulation, a strong tumor staining for both GRP78-binding phage clones was observed (FIG. 6) 630/650 640/650, whereas only background staining was detected in the control organs 630/660 630/670 640/660 640/670. In addition, control phage was not detected in tumors 610/650 620/650 or control organs such as the brain 660 and liver 670 (FIG. 6). These data show that human prostate cancer-derived tumor xenografts can be targeted by GRP78-binding phage vectors in vivo.

GRP78-Binding Phage Specifically Target Human Prostate Cancer Bone Metastases.

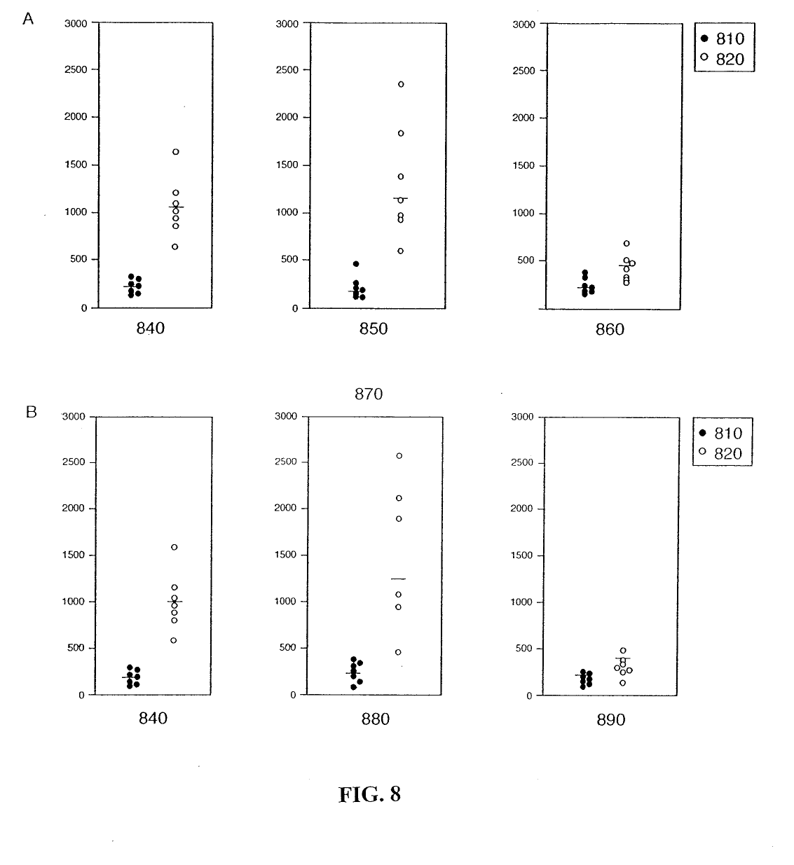

Since GRP78 expression was found to be high in bone metastases derived from prostate cancer patients, binding of the GRP78-binding phage to human prostate cancer bone metastases by phage overlay assays was tested. A strong staining with the GRP78-binding phage clones was observed, and marked inhibition when an anti-GRP78 antibody was added to the slide (FIGS. 8A and 8B), whereas no inhibition was observed with the control antibody 710 (FIG. 7A). To further confirm whether the GRP78-binding phage could inhibit the anti-GRP78 antibody staining, both GRP78-binding phage were incubated prior to the antibody (FIG. 7A), and a reduced antibody staining was observed. On the other hand, no staining inhibition was noted with the control phage 740 (FIG. 7A). GRP78 has been identified as a surface protein of tumor cells. In one embodiment, targeting peptides that bind GRP78 may be used to target GRP78 of prostate cancer cells to identify bone metastases. In another embodiment, targeting peptides that bind GRP78 may deliver pro-apoptotic agents to target GRP78 of cancer cells (e.g., prostate cancer) to prevent or treat bone metastasis.

GRP78-targeted pro-apoptotic peptides induce cell apoptosis. The efficacy of the WIFPWIQL (SEQ ID NO:6)-GG-_{D}(KLAKLAK)₂ (SEQ ID NO:11) 720 and WDLAWMFRLPVG (SEQ ID NO:7)-GG-_{D}(KLAKLAK)₂ (SEQ ID NO:11) 750 peptides in different GRP78-expressing prostate cancer cell lines, as verified by Annexin-V staining. Both peptides were toxic to DU145 cells (FIG. 7B) and to LnCap cells (data not shown) and induced apoptosis, while an equimolar mixture of uncoupled WIFPWIQL (SEQ ID NO:6) and _{D}(KLAKLAK)₂ 730 and WDLAWMFRLPVG (SEQ ID NO:7) and _{D}(KLAKLAK)₂ (SEQ ID NO:11) 760 SEQ ID NO:11 did not show any sign of toxicity. A dose dependent cell killing effect for the targeted peptides was also verified for both prostate cancer cell lines 720 750, while the uncoupled peptides did not affect cell survival (data not shown).

Treatment of cells and nude mice bearing DU145-derived human prostate carcinoma xenografts with WIFPWIQL (SEQ ID NO:6)-GG-_{D}(KLAKLAK)₂ (SEQ ID NO:11) 860 (FIG. 8A) and WDLAWMFRLPVG (SEQ ID NO:7)-GG-_{D}(KLAKLAK)₂ (SEQ ID NO:11) 890 (FIG. 8B). Individual tumor volumes are represented before (●) 810 and after (◯) 820 treatment for peptides WIFPWIQL (SEQ ID NO:6)-GG-_{D}(KLAKLAK)₂ (SEQ ID NO:11) 860 (FIG. 8A) and WDLAWMFRLPVG (SEQ ID NO:7)-GG-_{D}(KLAKLAK)₂ (SEQ ID NO:11) 890 (FIG. 8B). Controls used were vehicle alone and equimolar mixtures of unconjugated WIFPWIQL (SEQ ID NO:6) and _{D}(KLAKLAK)₂ (SEQ ID NO:11) for 850 (FIG. 8A) and WDLAWMFRLPVG (SEQ ID NO:7) and _{D}(KLAKLAK)₂ (SEQ ID NO:111) for 880 (FIG. 8B). Mean tumor volumes were significantly smaller (P<0.001, t-test) in mice treated with the coupled peptides, relative to the controls.

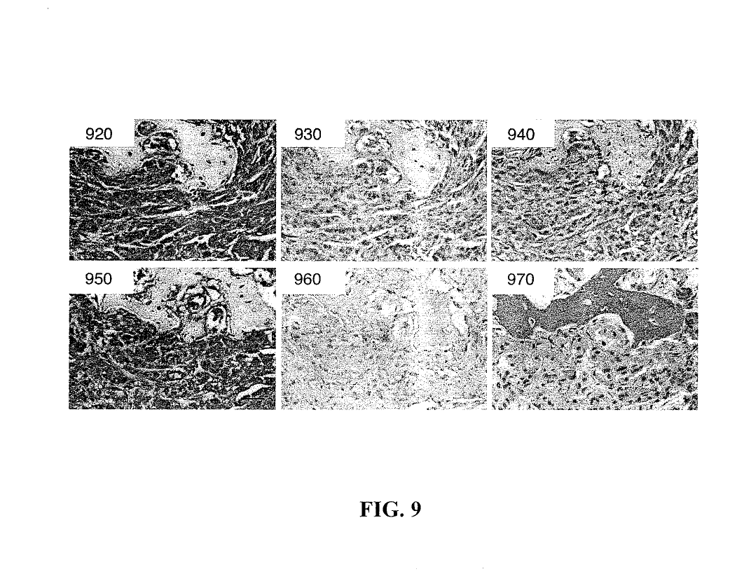

GRP78-targeted pro-apoptotic peptides prevent tumor growth in vivo. Given the results for apoptosis in cell cultures, the peptides were tested to see whether they have anti-cancer activity in vivo, using human prostate cancer xenografts. Mean tumor volume in the groups treated with the targeted peptides was 70% lower (P<0.001, t-test) than in the controls (FIG. 9) 930 940. Individual tumor volumes before 960 and after 930 940 treatment are represented and no peptide 950 and control phage 920.

Inhibition of GRP78-binding phage clones staining by anti-GRP78 antibody. Serial tissue sections of bone metastases from human prostate cancer were incubated with an anti-GRP78 antibody prior to adding the WIFPWIQL (SEQ ID NO:6)-phage (FIG. 9) 930, WDLAWMFRLPVG (SEQ ID NO:7)-phage (FIG. 9) 940 and negative control phage 920 to the sections. Strong staining was observed when the phage was used without antibody (FIG. 10) 1030 1060 and with the control antibody 1020 1050. In contrast, a marked reduction in phage staining was observed when the anti-GRP78 antibody 930 940 was used. Scale bar, 100 μm.

Inhibition of Anti-GRP78 Antibody Staining by GRP78-Binding Phage Clones.

Serial tissue sections of bone metastases from human prostate cancer were incubated with the WIFPWIQL (SEQ ID NO:6)-phage 930, WDLAWMFRLPVG (SEQ ID NO:7)-phage 940 and negative control phage 920 prior to adding an anti-GRP78 antibody to the sections. Strong staining was observed when the anti-GRP78 antibody was used without phage 950, compared to a negative control antibody 960 with the same isotype and at the same concentration. Pre-incubation with WIFPWIQL (SEQ ID NO:6)-phage 1010 or WDLAWMFRLPVG (SEQ ID NO:7)-phage 1040 inhibited the staining by the anti-GRP78 antibody whereas a negative control phage (displaying no peptide) did not affect the staining of the anti-GRP78 antibody. An eosin staining of the tumor is shown in 970. Scale bar, 100 μm.

C. Discussion

Recent evidence suggests that heat shock proteins present on the surface of tumor cells may serve as molecular targets for diagnosis and/or targeted therapy. First, global profiling of the cell surface proteome of tumor cells disclosed an abundance of chaperone heat shock proteins. Second, fingerprinting the repertoire of circulating antibodies from cancer patients with phage display random peptide libraries has identified a conformational mimic motif of one such heat shock protein family member, GRP78. Third, a decrease in α2-microglobulin predicts metastatic prostate cancer, therefore it may also suggest an increase in GRP78, as metastatic prostate cancer and the immune response to GRP78 are associated. Interestingly, the humoral response elicited against the GRP78 mimic motif or against the native GRP78 was shown to have a strong correlation with the development of androgen-independent disease and shorter overall survival in a large population of prostate cancer patients. These observations led to efforts to test and establish GRP78 on the tumor cell membrane as a translational target for therapeutic intervention in the context of human prostate cancer.

Phage clones expressing GRP78-binding peptides by cloning the inserts WIFPWIQL (SEQ ID NO:6) and WDLAWMFRLPVG (SEQ ID NO:7) into a phage construct were generated. The ability of GRP78-binding phage in vitro was evaluated. In addition to the significant higher binding of phage to GRP78 than to related and unrelated control proteins, the synthetic WIFPWIQL (SEQ ID NO:6) and WDLAWMFRLPVG (SEQ ID NO:7) peptides inhibit binding of the corresponding phage in a dose-dependent manner, demonstrating specificity for the interaction.

The next step in the development of the ligand-receptor system was to evaluate binding to GRP78 expressed in the membrane of human prostate cancer-derived cells. It was previously shown that hydrophobic passage through an organic phase is an efficient method for selection and quantitation of phage binding. Using the BRASIL method, WIFPWIQL (SEQ ID NO:6)-phage and WDLAWMFRLPVG (SEQ ID NO:7)-phage clones targeted GRP78 expressed on the membrane of the prostate cancer cells were targeted. The protein-protein interaction in living cells was specific, as phage binding to the cells was inhibited by an anti-GRP78 polyclonal antibody, by GRP78 in solution, and by the synthetic cognate peptides.

To test whether the GRP78-binding phage could target tumor xenografts derived from human prostate cancer in vivo, the phage constructs and controls intravenously into tumor-bearing nude mice were administered. At a delayed point after systemic administration (24 h), it was observed that marked localization of GRP78-binding phage into the tumor xenografts, with barely noticeable phage localization to the control organs. Given the capacity of the DU145 cells in culture to internalize GRP78-targeting phage (data not shown), the prolonged circulation time of the phage, and the staining pattern observed in vivo, it is likely that GRP78 mediated phage internalization occurred in the tumor cells, suggesting that GRP78 aptamers can promote targeting of prostate cancer-derived tumors even under in vivo conditions.

Having confirmed the tumor-targeting ability of the GRP78-binding phage clones in a mouse model, it was evaluated whether the WIFPWIQL (SEQ ID NO:6) and WDLAWMFRLPVG (SEQ ID NO:7) peptides would bind to human prostate cancer bone metastases. By using phage overlay assays, sections from human bone metastases showed stronger staining when exposed to GRP78-binding phage clones than to fd-tet phage. It may be that these results reflect the differential expression pattern of the target in metastatic androgen-independent prostate cancer. It was shown that the GRP78-binding phage clones could specifically compete the staining of an anti-GRP78 antibody, presumably due to the relatively large size of phage particles that can disrupt the protein-antibody interaction. Similarly, an anti-GRP78 antibody specifically blocked the staining of the GRP78-binding phage. Taken together, these results suggest specificity for phage binding to human bone metastases.

The efficacy of the GRP78-targeted peptides to deliver a pro-apoptotic motif to human prostate cancer cells was tested. A low concentration of the coupled peptides was considerably toxic to DU145 cells. Progressive cellular damage was detected 2 hours after the addition of the peptides. After 24 hours, cells showed profound morphologic alterations, and apoptosis was detected in almost 100% of the cells. In contrast, an equimolar mixture of uncoupled GRP78-targeted peptides and _{D}(KLAKLAK)₂ (SEQ ID NO:11) (negative control) did not induce any toxicity to the cells.

Most interestingly, a significant reduction in tumor volume when prostate carcinoma xenografts were treated with the targeted peptides was found, and no sign of toxicity was observed. Tumor volume was on average 30% that of the control groups for both GRP78-targeting peptides. Collectively, these data show that GRP78 peptides may be used for targeted therapy against prostate cancer. Because GRP78 expression is induced in conditions present in solid tumors such as cellular stress and hypoxia, the functional or immunological importance, if any, of interfering with this chaperone heat shock protein remains an additional possibility to be explored.

### Example 2

### Adipose Tissue Targeting

A. Material and Methods

Experimental animals. C57BL/6 mice were purchased from Harlan Teklad (Indianapolis, Ind.); ob/ob mice (stock 000632) were purchased from Jackson Laboratories (Bar Harbor, Me.). All animal experiments involved standard procedures approved by The University of Texas M. D. Anderson Cancer Center and Baylor College of Medicine.

In vivo phage library selection. In vivo phage-display screening of a CX₇C library (C, cysteine; X, any amino acid residue) for fat-homing peptides was performed as described. In each biopanning round, an adult ob/ob female mouse was injected intravenously (i.v) via tail vein with 10¹⁰ transducing units (TU) of the library. Phage (˜300 TU/g in round 1 increased to ˜10⁴/g TU in round 3) were recovered after 5 min of circulation from subcutaneous fat, and bulk-amplified for each subsequent round. Phage amplified after the third round of panning was enriched for fat-specific binders by adapting an in vivo subtraction step: a lean C57BL/6 female was injected i.v. with 10⁹ TU of phage selected in round 3. After 5 min, the unbound phage were recovered from circulation and amplified for the fourth and final round of biopanning in an ob/ob female mouse.

Histopathology. Staining of formalin-fixed, paraffin-embedded mouse tissue sections was performed as described. For phage-peptide immunolocalization, 10¹⁰ TU of CKGGRAKDC (SEQ ID NO:4)-phage or a control insertless phage was administered intravenously. Phage immunohistochemistry was performed by using a rabbit anti-fd phage antibody B-7786 (Sigma, St Luis, Minn.) at 1:1,000 dilution. For in vivo peptide homing validation, stock solutions of 5-Carboxyfluorescein (fitc)-conjugated CKGGRAKDC (SEQ ID NO:4) and CARAC (SEQ ID NO:9) chemically synthesized, cyclized, and HPLC-purified to 99% purity by AnaSpec (San Jose, Calif.) were prepared by dissolving the lyophilized peptides in DMSO to a concentration of 20 mM. Peptide-fitc solutions in phosphate buffer saline (PBS; 10 μl of 1 mM) were administered i.v. 5 min prior to tissue collection. For blood vessel localization, rhodamine-conjugated lectin-I (RL-1102, Vector Laboratories, Burlingame, Calif.) was co-administered (10 μl of 2 mg/ml). Apoptosis was detected by using standard TUNEL immunohistochemistry. Prohibitin immunolocalization was performed with polyclonal rabbit antibody RDI-PROHIBITabr (Research Diagnostics Inc., Flanders, N.J.) at 1:50 dilution. Iimmunohistochemistry was performed with the LSAB+ peroxidase kit (DAKO, Carpinteria, Calif.). Images were captured digitally with an Olympus IX70 microscope.

Fat resorption and metabolic analysis. High-calorie diet for obesity induction (TD97366: 25.4% fat, 21.79% protein, 38.41% carbohydrate) was purchased from Harlan Teklad. Mice have been pre-fed with TD97366 prior to treatment to induce diet-related obesity until a weight greater than 45 g was acquired. Stocks of CKGGRAKDC (SEQ ID NO:4)-GG-_{D}(KLAKLAK)₂ (SEQ ID NO:11), CGDKAKGRC (SEQ ID NO:10)-GG-_{D}(KLAKLAK)₂ (SEQ ID NO:1), _{D}(KLAKLAK)₂, CARAC (SEQ ID NO:9)-GG-_{D}(KLAKLAK)₂ (SEQ ID NO:1), and CKGGRAKDC (SEQ ID NO:4) chemically synthesized, cyclized, and HPLC-purified to 99% purity (AnaSpec) were prepared by dissolving lyophilized peptides in DMSO to a concentration of 65 mM. For each peptide, 100 nM of peptide stock dissolved in PBS was administered in the subcutaneous tissue of the back of C57BL/6 males daily for 4 weeks. Mouse weight, body temperature, and food and water consumption were monitored weekly. Tissue lipids were measured as described. Mice were fasted for 10 h for analyses of serum lipids and GTT. The following kits were used: NEFA-C (WAKO Chemicals, Richmond, Va.) for free fatty acids; GPO-TRINDER (Sigma) for glycerol and triacyl glyceride; rat Insulin ELISA (Crystal Chemical, Houston, Tex.) for insulin; TRINDER 100 (Sigma) for glucose; cholesterol E kit (WAKO chemicals) for cholesterol, and Quantikine M Immunoassay (R&D Systems, Minneapoli, Minn.) for leptin. Oxygen consumption and heat generation of fasting mice were measured for 24 h by indirect calorimetry with OXYMAX (Columbus Instruments, Columbus, Ohio). To quantify spontaneous activity, 8 mice (2 mice per cage) were placed in automated photocell activity cages (AccuScan Instruments, Columbus, Ohio). Mice were habituated to the activity cages prior to testing on the experimental day. Horizontal locomotor activity was computer-monitored as the number of infrared beam breaks, which were detected for the period of 1 h and recorded every hour for the duration of the test (14 h) by using a Versamax Analyser. Recordings were taken during the dark cycle lasting 6 pm-8 am with water and food freely available.

Characterization of the CKGGRAKDC (SEQ ID NO:4)-prohibitin interaction. In vitro biotinylation of the vasculature and extraction of membrane proteins was performed as described. For expression and immobilization of gst fusions on the column, GST-Bind Kit (Novagen, Madison, Wis.) was used. Sepharose 4B (Amersham, Piscataway, N.J.) was loaded with 100 μg of purified gst fusions desalted with Amicon filters (Millipore, Bedford, Mass.). Fat membrane proteins bound to gst fusions were eluted with 1 mM fitc-peptides. For immunoblotting, anti-prohibitin polyclonal antibody RDI-PROHIBITabr (RDI) diluted to 1:1,000 was used. Sepharose 100EAH (Amersham) was loaded with 5 mg of CKGGRAKDC (SEQ ID NO:4)-GG-_{D}(KLAKLAK)₂ (SEQ ID NO:11), or CARAC (SEQ ID NO:9)-GG-_{D}(KLAKLAK)₂ (SEQ ID NO:11). Chromatography was performed in an Econo Pump/Econo-Column Adaptor set (Biorad, Hercules, Calif.). Bound proteins were eluted by 100 mM glycine (pH 2.5). MALDI-TOF mass spectrometry was performed at a Core Facility of Baylor College of Medicine. To evaluate interaction of phage-CKGGRAKDC (SEQ ID NO:4) with prohibitin in vitro, binding of 10⁹ TU CKGGRAKDC (SEQ ID NO:4)-displaying or control (fd-Tet) phage to recombinant gst-fused prohibitin was performed as described. Anti-prohibitin polyclonal antibody RDI-PROHIBITabr diluted 1:10 was incubated with the immobilized protein for 1 hr at RT prior to adding phage to test whether the binding would be blocked. Phage binding was assayed by infection of the host E. Coli and quantification of TU recovered from the wells.

B. Results

Most anti-obesity agents are based on altering energy balance pathways and appetite by acting on receptors in the brain. In addition, some drugs of this class (such as fenfluramine) have been withdrawn from the market due to unexpected toxicity. Recent attempts to develop compounds that inhibit absorption of fat through gastrointestinal tract (such as Orlistat) may improve anti-obesity treatment. Still, even the most effective drugs can only reduce weight by up to 5% and strict dieting is required for further weight loss.

Proliferation of tumor cells depends on new blood vessel formation (angiogenesis) that accompanies malignant progression. Anti-cancer therapy with angiogenesis inhibitors or cytotoxic agents targeted to the vasculature of tumors are currently being evaluated in clinical trials. While white fat is a non-malignant tissue, it has the capability to quickly proliferate and expand. Histological evaluation of adipose tissue reveals that fat is highly vascularized: multiple capillaries make contacts with every adipocyte, suggesting the importance of blood vessels for maintenance of the tissue mass. It was recently shown that non-specific angiogenesis inhibitors may prevent the development of obesity in mice, and regulation of hepatic tissue mass by angiogenesis has also been reported. Targeting existing blood vessels in white fat could result in adipose tissue ablation. Therefore, peptide ligands that bind to receptors in white fat vasculature were targeted. Targeted delivery of a chimeric peptide containing a pro-apoptotic sequence to the fat vasculature of obese mice resulted in obesity reversal and metabolic normalization without change in food intake.

CKGGRAKDC homes to white fat in mice. About 5% of the clones identified in the screen. By intravenously administering this clone into ob/ob mice, it was shown that CKGGRAKDC (SEQ ID NO:4)-displaying phage accumulated in subcutaneous fat approximately 150-fold over the background observed for a negative control insertless phage; this quantification of phage recovery was accomplished by standard counting of phage transducing units per gram of tissue. Next, the tropism of CKGGRAKDC (SEQ ID NO:4)-phage for the target tissue by immunohistochemistry was confirmed: CKGGRAKDC (SEQ ID NO:4)-phage showed localization to the vasculature of subcutaneous and peritoneal white fat (FIGS. 11A and 11B), whereas the control phage was undetectable in blood vessels of white fat (FIGS. 11C and 11D). In contrast, in brown fat (FIGS. 11E and 11F) and in several other control organs (liver, pancreas, skeletal muscle, lung, and kidney; data not shown) of CKGGRAKDC (SEQ ID NO:4)-phage-injected mice, staining was not detectable above the background levels of control insertless phage.

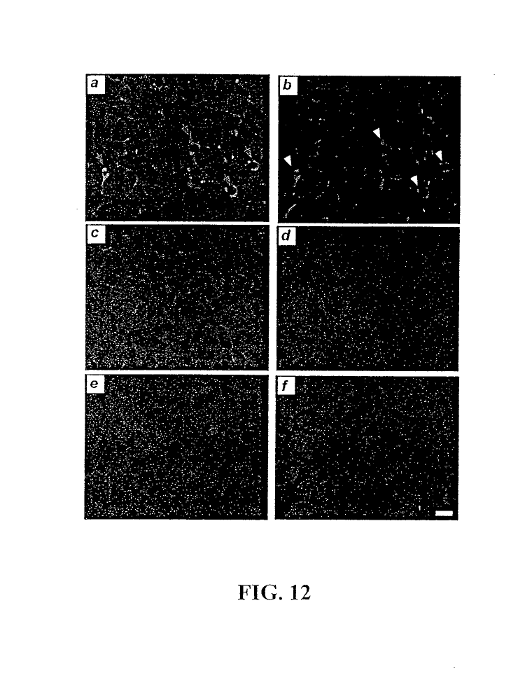

The genetic ob/ob model is not representative of the vast majority of obese patients because the mutation in mouse leptin is only rarely found in the context of human obesity. Thus, to evaluate whether the CKGGRAKDC (SEQ ID NO:4) peptide would target adipose tissue in mice irrespective of the obesity model, whether the CKGGRAKDC (SEQ ID NO:4) motif also homes to fat in wild-type mice was tested. In addition, to confirm that targeting of the CKGGRAKDC (SEQ ID NO:4) motif to the fat vasculature occurs when the corresponding synthetic peptide is outside of the context of the phage, the in vivo distribution of intravenously administered soluble CKGGRAKDC (SEQ ID NO:4) peptide linked to fluorescein (fitc) at its C-terminus was determined. As in ob/ob mice, CKGGRAKDC (SEQ ID NO:4)-fitc specifically localized to and was internalized by blood vessels of subcutaneous and peritoneal white fat in wild-type mice (FIGS. 12A and 12B). In contrast, neither of the two negative control peptides tested (unrelated CARAC (SEQ ID NO:9)-fitc or scrambled CGDKAKGRC (SEQ ID NO:10)-fitc) was detectable in the white fat vasculature (FIG. 12C). Moreover, no CKGGRAKDC (SEQ ID NO:4)-fitc homing was observed in blood vessels of brown fat (FIGS. 12D and 12F) or liver (FIG. 12E) and other control organs tested. Finally, the presence of CKGGRAKDC (SEQ ID NO:4)-fitc, but not of scrambled CGDKAKGRC (SEQ ID NO:110)-fitc peptide was shown in isolated ex-vivo blood vessels from the white fat tissue.

The in vivo localization studies presented here show that CKGGRAKDC (SEQ ID NO:4) targets the white adipose vasculature without a detectable preference for any particular anatomical white fat depot. The uptake of CKGGRAKDC (SEQ ID NO:4)-fitc by the endothelium of white fat tissue suggests that the motif is preferentially internalized by a receptor in the adipose vasculature that could serve for targeted delivery of therapeutic compounds to fat.

Designing a fat vasculature-targeted chimeric proapoptotic peptide. Next, whether white fat tissue mass could be controlled by targeted destruction of fat vasculature was studied. The amphipatic peptide sequence KLAKLAKKLAKLAK (SEQ ID NO:11), designated (KLAKLAK)₂ (SEQ ID NO:11), which disrupts mitochondrial membranes upon receptor-mediated cell internalization and causes programmed cell death, has been used for targeted apoptosis induction in tumor blood vessels. Herein a synthetic peptide composed of two functional domains was produced: one the white fat vasculature homing motif CKGGRAKDC (SEQ ID NO:4) and the other the D-enantiomer _{D}(KLAKLAK)₂ (SEQ ID NO:11), which is resistant to proteolysis; these two functional domains were linked by a glycinylglycine bridge. The resulting prototype fat-targeted pro-apoptotic chimeric peptide, termed CKGGRAKDC (SEQ ID NO:4)-GG-_{D}(KLAKLAK)₂ (SEQ ID NO:11), contained 25 amino acid residues synthesized by conventional peptide chemistry (“Merrifield synthesis”).

White fat ablation with CKGGRAKDC (SEQ ID NO:4)-GG-_{D}(KLAKLAK)₂ (SEQ ID NO:11) To determine whether CKGGRAKDC (SEQ ID NO:4)-GG-_{D}(KLAKLAK)₂ (SEQ ID NO:11) could be used for therapeutic destruction of fat vasculature, a non-genetic mouse obesity model was used. Cohorts of wild-type mice, in which obesity had been induced by a high-calorie diet received daily subcutaneous (s.c.) doses of the synthetic CKGGRAKDC (SEQ ID NO:4)-GG-_{D}(KLAKLAK)₂ (SEQ ID NO:11) peptide. High-calorie diet feeding continued throughout the experiment. CKGGRAKDC (SEQ ID NO:4)-GG-_{D}(KLAKLAK)₂ (SEQ ID NO:11) administration not only prevented obesity development, but also caused a rapid decrease in white fat mass and obesity reversal (FIGS. 13A and 13B). Four weeks into the treatment, mice lost an average of 15 g (over 30%) in weight (FIG. 13A) and displayed a reduction in body fat content. Epididymal fat pad size decreased by more than 3-fold compared with controls: 0.6±0.02 g in treated versus 2.1±0.03 g in control mice (P<0.001; FIG. 13B). In contrast, control mice receiving an equimolar mixture of the CKGGRAKDC (SEQ ID NO:4) peptide and untargeted _{D}(KLAKLAK)₂ (SEQ ID NO:11) peptide continued to develop worsening obesity (FIGS. 13A and 13B).

To explore the molecular and biochemical mechanism(s) of fat resorption, the serum lipids in the two groups of animals were measured. Mice treated with CKGGRAKDC (SEQ ID NO:4)-GG-_{D}(KLAKLAK)₂ (SEQ ID NO:11) displayed a progressive elevation in the serum level of free fatty acids (22% increase at four weeks) and glycerol (24% increase at four weeks) as compared with the levels in control peptide-treated mice (FIG. 13C). Thus, treatment appeared to have activated lipolysis in obese mice. However, the serum triglyceride and cholesterol concentrations were only marginally higher in treated animals than in the control (FIG. 13C). Histological analysis of tissues from mice after four weeks of treatment revealed that mice treated with control peptides had fat deposits in liver, whereas those treated with the therapeutic peptide regained normal histological appearance with reduced fat infiltration in the liver (FIG. 13C). Such reversal of hepatic steatosis (“fatty liver”) was confirmed by quantification of extracted lipids: livers of treated mice contained approximately half the amount of lipid compared with that of the livers from control mice (FIG. 13E; P<0.0075). Lipid contents of skeletal muscles (soleus and gastrocnemius) were also lower in treated than in control animals (FIG. 13E; P<0.02). A reduction in the serum leptin level detected by the four weeks of treatment (FIG. 13E) is consistent with white fat resorption and the resulting decreased number of adipocytes. Food consumption (high-calorie diet) was not different between mice treated with the therapeutic peptide and those treated with the control peptide (FIG. 13G).

The anti-obesity effects of CKGGRAKDC (SEQ ID NO:4)-GG-_{D}(KLAKLAK)₂ (SEQ ID NO:11) in several additional experiments was reproduced, which included different untargeted _{D}(KLAKLAK)₂ (SEQ ID NO:11) peptides as negative controls along with mock saline administration (data not shown). Neither a scrambled CGDKAKGRC (SEQ ID NO:10)-GG-_{D}(KLAKLAK)₂ (SEQ ID NO:11) peptide nor the unrelated CARAC (SEQ ID NO:9)-GG-_{D}(KLAKLAK)₂ (SEQ ID NO:11) peptide induced weight loss. The effectiveness of CKGGRAKDC (SEQ ID NO:4)-GG-_{D}(KLAKLAK)₂ (SEQ ID NO:11) in another non-genetic mouse model of obesity was tested: regular diet-fed wild-type mice that had became obese due to their old age. As observed for the diet-induced obesity model, targeting of _{D}(KLAKLAK)₂ (SEQ ID NO:11) to fat with CKGGRAKDC (SEQ ID NO:4) resulted in a reduction in body mass at a rate of ˜10% per week without detectable toxicity. The peptide effect was dose-dependent in the range of 50-100 nM/day. Upon discontinuation of treatment, mice slowly re-gained their weight at a rate of ˜0.05 g/day.

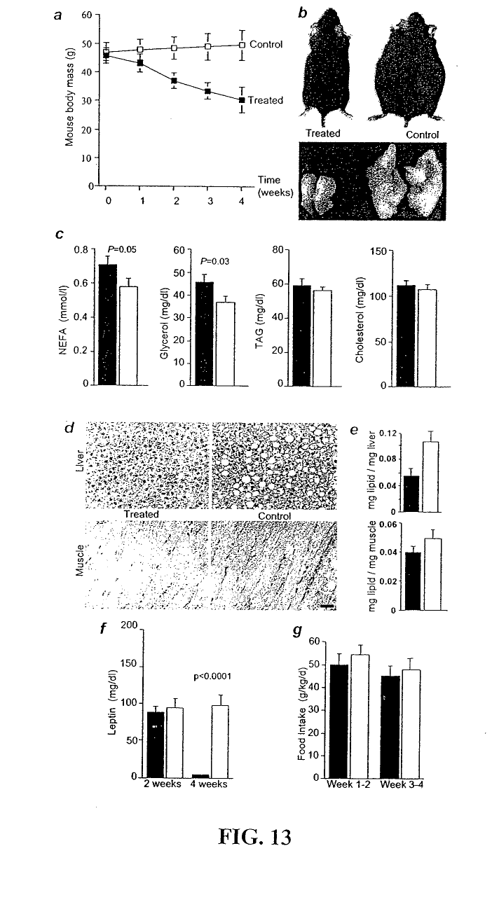

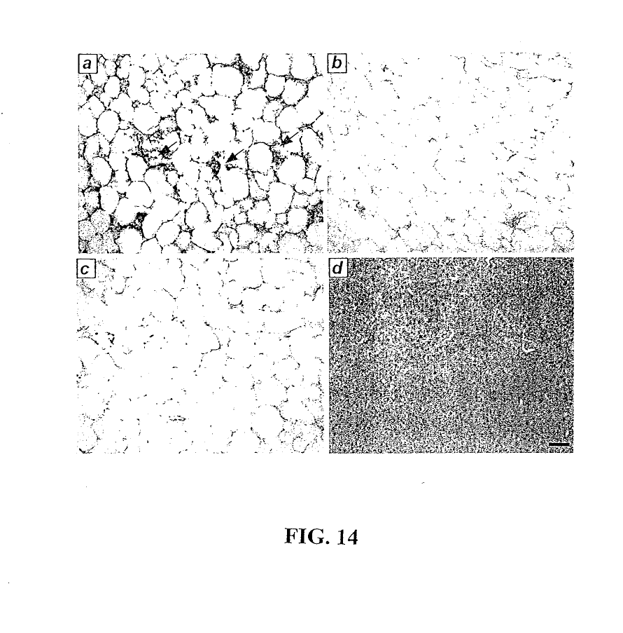

CKGGRAKDC (SEQ ID NO:4)-GG-_{D}(KLAKLAK)₂ (SEQ ID NO:11) causes white fat resorption by targeted apoptosis. In both diet-induced and age-related obesity, fat resorption resulting from treatment with CKGGRAKDC (SEQ ID NO:4)-GG-_{D}(KLAKLAK)₂ (SEQ ID NO:11) was evident upon anatomic examination. Gross morphological evaluation of mouse organs post-mortem revealed that both subcutaneous and visceral fat depots were reduced by CKGGRAKDC (SEQ ID NO:4)-GG-_{D}(KLAKLAK)₂ (SEQ ID NO:11) treatment (FIG. 14B). Histopathological analysis of white adipose tissue from treated mice revealed vascular apoptosis (FIG. 14A) induced by treatment with the peptide CKGGRAKDC (SEQ ID NO:4)-GG-_{D}(KLAKLAK)₂ (SEQ ID NO:11) but not with control peptides (FIG. 14B). In contrast, control organs, such as liver, appeared grossly and microanatomically normal, and apoptosis was not detected in the control tissues (FIG. 14D).

Metabolic effects of peptide-mediated fat ablation. Having shown the physiological consequences of fat ablation with CKGGRAKDC (SEQ ID NO:4)-GG-_{D}(KLAKLAK)₂ (SEQ ID NO:11) peptide in mice and keeping in mind that the food consumption between treated and control groups was indistinguishable (FIG. 13), then indirect calorimetry to measure metabolic parameters in the treated and control mice after one and four weeks of treatment was used. Total oxygen consumption and carbon dioxide production were found to be increased after four weeks of CKGGRAKDC (SEQ ID NO:4)-GG-_{D}(KLAKLAK)₂ (SEQ ID NO:11) treatment under both fed and starving conditions to the levels normally observed in lean mice. The increased metabolism was also reflected by an increase in heat production after four weeks of treatment, which approached heat expenditure observed in lean mice. Also, a decrease in the respiratory exchange ratio (RER) measured in mice under fed conditions (0.77 for treated mice versus 0.83 for control peptide-treated mice; P<0.007) and under starving conditions at 4 weeks was detected. The decreased respiratory quotient in animals treated with the fat-targeting peptide indicates that the increase in metabolic rate upon treatment results, at least in part, from an up-regulation of the metabolism of lipid substrates.

Next, to rule out the possibility that the treatment induced an increased physical activity, spontaneous movements of obese mice treated with therapeutic or control peptides were measured. Locomotor activity of mice (treated with anti-obesity peptide-treated, n=8; treated with control peptide, n=8; and untreated isogenic lean, n=8) was monitored and assessed by computer-assisted counting of infrared light beam interruptions in activity cages. The activity of the mice after one week of treatment and again after four weeks of treatment were compared. No increase in the physical activity of treated mice at both time points was detected.

Finally, glucose tolerance test (GTT) on the two groups of obese mice that were measured, prior to treatment, had developed increased adiposity, insulin resistance, and glucose intolerance as a result of their high-fat diet feeding. Four weeks after the initiation of treatment, control peptide-treated mice displayed a diabetic curve with fasting hyperglycemia as well as elevated serum glucose levels at different time points following an intraperitoneal (i.p.) glucose load. In contrast, CKGGRAKDC (SEQ ID NO:4)-GG-_{D}(KLAKLAK)₂-(SEQ ID NO:11) treated mice had normal fasting serum glucose and improved serum glucose levels at all time points during the test. Furthermore, control mice exhibited severe hyperinsulinemia throughout the 120-minute GTT, whereas the serum insulin values were reduced in mice that received the fat-targeted peptide.

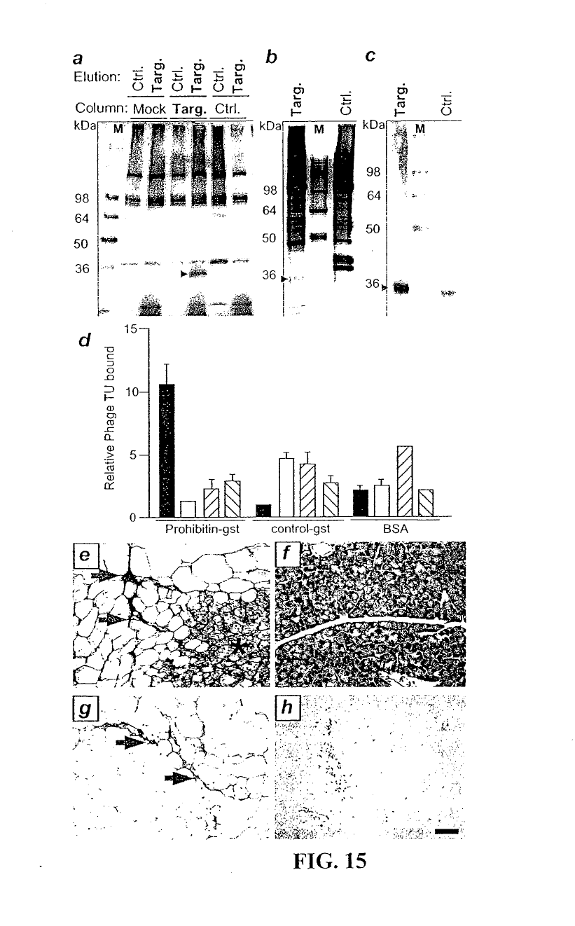

CKGGRAKDC (SEQ ID NO:4) Targets prohibitin in white fat. To identify the vascular receptor of CKGGRAKDC (SEQ ID NO:4), affinity chromatography was used to identify cell membrane proteins that bind to immobilized CKGGRAKDC (SEQ ID NO:4). Ob/ob mice were perfused with biotin, extracted membrane proteins from white adipose tissue, and isolated proteins specifically binding to beads coated with the recombinant fusion protein CKGGRAKDC (SEQ ID NO:4)-glutathione transferase (gst). A specific band of ˜35 kDa size was eluted from CKGGRAKDC (SEQ ID NO:4)-gst-coated beads with the CKGGRAKDC (SEQ ID NO:4)-fitc peptide and detected it by anti-biotin immunoblotting (FIG. 15A). This protein was neither eluted from CKGGRAKDC-gst-loaded beads with CVMGSVTGC (SEQ ID NO:12)-fitc control (another fat-homing peptide isolated in the phage display selection) nor was it eluted with CKGGRAKDC (SEQ ID NO:4)-fitc from unloaded beads, or beads loaded with the recombinant fusion protein CVMGSVTGC (SEQ ID NO:12)-gst (FIG. 15A). To identify the CKGGRAKDC-(SEQ ID NO:4)-binding protein (and to show that this 35 kDa protein was not present exclusively in ob/ob mice), purification of CKGGRAKDC-(SEQ ID NO:4)-binding membrane proteins from white adipose tissue of wild-type mice was used. For this large-scale purification, the synthetic peptide CKGGRAKDC (SEQ ID NO:4)-GG-_{D}(KLAKLAK)₂ (SEQ ID NO:11), was immobilized which was proven functional in vivo, to minimize the co-isolation of proteins nonspecifically binding to the relatively large gst domain of CKGGRAKDC (SEQ ID NO:4)-gst. After pre-clearing the adipose membrane extract on a control CARAC (SEQ ID NO:9)-GG-_{D}(KLAKLAK)₂-loaded column, the cleared extract to the CKGGRAKDC (SEQ ID NO:4)-GG-_{D}(KLAKLAK)₂ column was applied and then performed acidic elution of bound proteins. Consistent with the gst chromatography results, the 35 kDa protein was specifically detected in the eluate from the CKGGRAKDC (SEQ ID NO:4)-GG-_{D}(KLAKLAK)₂ (SEQ ID NO:11) column, but not from the control column (FIG. 15B).

Mass spectrometry analysis of the 35 kDa fraction of the eluate unequivocally (confidence 2.067e+004) identified the protein as prohibitin. To confirm that the isolated protein is in fact prohibitin by immunoblotting the eluates from the fat targeting CKGGRAKDC (SEQ ID NO:4)-GG-_{D}(KLAKLAK)₂ peptide and unrelated control CARAC (SEQ ID NO:9)-GG-_{D}(KLAKLAK)₂ (SEQ ID NO:11) peptide columns (FIG. 15B) with an anti-prohibitin antibody were performed (FIG. 15C). Then the interaction of the CKGGRAKDC (SEQ ID NO:4) peptide and prohibitin at the protein-protein level was shown directly by using an in vitro ligand-receptor binding assay (FIG. 15D). The CKGGRAKDC (SEQ ID NO:4)-displaying phage bound to immobilized prohibitin 8-fold relative to a control insertless phage. Binding of CKGGRAKDC (SEQ ID NO:4)-displaying phage to a control gst fusion and bovine serum albumin (BSA) used as negative controls were at background binding of the insertless phage to the immobilized proteins (FIG. 15D). Moreover, anti-prohibitin polyclonal antibody blocked the binding of CKGGRAKDC (SEQ ID NO:4)-displaying phage to prohibitin but did not affect the control phage binding, indicating specificity of the interaction (FIG. 15D). These results indicate that the CKGGRAKDC (SEQ ID NO:4) motif targets prohibitin in the blood vessels of fat.

The expression of prohibitin in the vasculature was explored by using an affinity-purified polyclonal antibody. In addition to mitochondrial expression, previously shown with a monoclonal antibody for a number of organs, including brown fat (FIG. 15E), a high level of prohibitin expression in the vasculature of white adipose tissue was detected (FIG. 15E). Consistent with the pattern of in vivo distribution of the CKGGRAKDC (SEQ ID NO:4) motif, prohibitin was not expressed in blood vessels of control tissues (FIG. 15F). Mouse and human prohibitins vary by a single amino acid residue and the antibody also recognized the human protein in the blood vessels of human white adipose tissue (FIG. 15G). Finally, prohibitin may be a white fat vascular differentiation marker because it is undetectable in the vasculature of human anaplastic liposarcomas, and poorly-differentiated malignant tumors derived from white adipose tissue (FIG. 15H). In one embodiment, a peptide that specifically binds to adipose vascular tissue may be used to target adipose tissue for diagnosis. In another embodiment, a peptide that specifically binds to adipose vascular tissue may be used to deliver an agent such as a pro-apoptotic agent to induce apoptosis in adipose cells. In yet another embodiment, a peptide that specifically binds to prohibitin in adipose vascular tissue may be used to diagnose or to treat adipose tissue such as targeted pro-apoptotic agent delivery. These methods may be used to reduce fat for weight control in a subject by eliminating adipose tissue.

An approach to treatment of obesity based on targeted apoptosis induction in blood vessels of adipose tissue. Resorption of white fat was shown to lead to weight loss by activation of lipid metabolism and increased energy expenditure, as reflected by oxygen consumption and heat generation.

The data presented here indicate that a protein complex containing prohibitin, a membrane-associated protein with an as yet poorly defined function is the homing target of the CKGGRAKDC SEQ ID NO:4 peptide in white adipose vasculature. Prohibitin is thought to regulate cell survival and growth at several levels: as a mitochondrial membrane chaperone and through interaction with cell cycle proteins in the nucleus. Prohibitin has also been isolated from the cell membrane but its function as a transmembrane signaling receptor is still elusive. Immunohistochemical analysis with an anti-prohibitin polyclonal antibody shows expression of prohibitin in the membrane of endothelial cells in white adipose tissue. This study establishes a role for prohibitin as an endothelial cell surface receptor. Homing of the CKGGRAKDC (SEQ ID NO:4) peptide to blood vessels of white fat is likely based on targeting of the prohibitin receptor complex via increased accessibility of this receptor to the circulating ligand due to cell membrane localization in the white adipose vasculature.

Reversal of the diet-induced obesity was associated with up-regulation of lipid turnover and increased metabolic rate. The metabolic profile observed in mice treated with fat vasculature-targeted pro-apoptotic peptide recapitulates that of non-obese mice. These observations are reminiscent of the recent results reported for obesity prevention (but not reversal) with non-selective angiogenesis inhibitors. In some mouse models, adipose tissue ablation results in adverse physiological consequences whose severity correlates with the extent of fat loss. Accumulation of fat in other tissues (steatosis) and in the circulation (dyslipidemia), as well as diabetes mellitus, are well recognized complications in models of severe white fat deficiency. However, in response to the treatment described here, adverse physiological consequences despite the observed weight loss was not detected. The animals appear to have normalized their energy expenditure mainly through reversal of fat metabolism to a higher (closer to normal) rate. Moreover, upon adipose tissue resorption, no fat accumulation in other organs was detected; on the contrary, the steatotic liver phenotype of obese animals was reversed by treatment. Finally, obesity treatment by resorption of fat vasculature resulted in an improvement in glucose tolerance and insulin resistance. The absence of dyslipidemia in the treated mice in this study may be due to the relatively slow and incomplete fat ablation, as the treatment led to a normal body habitus with typical normal amounts—but not total absence—of body fat. Consistently, recent studies in PTP1B and fat-specific insulin receptor knockout mice demonstrated that low body fat may be maintained, despite normal food intake, without detectable side effects.

Taken together, adipose vascular targeting agents may result in rapid weight loss without affecting food intake and, apparently, avoiding some of the side effects observed in other mouse models. Given that the targeting system described here may also be functional in the context of human obesity, translation into potential clinical applications might be feasible.

All of the compositions, methods and apparatus disclosed and claimed herein can be made and executed without undue experimentation in light of the present disclosure. While the compositions and methods of this invention have been described in terms of preferred embodiments, it are apparent to those of skill in the art that variations maybe applied to the compositions, methods and apparatus and in the steps or in the sequence of steps of the methods described herein without departing from the concept, spirit and scope of the invention. More specifically, it are apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,472,509
U.S. Pat. No. 5,021,236
U.S. Pat. No. 5,206,347
U.S. Pat. No. 5,223,409
U.S. Pat. No. 5,401,511
U.S. Pat. No. 5,492,807
U.S. Pat. No. 5,603,872
U.S. Pat. No. 5,622,699
U.S. Pat. No. 5,670,312
U.S. Pat. No. 5,705,610
U.S. Pat. No. 5,840,841
U.S. Pat. No. 5,889,155
U.S. Pat. No. 6,068,829
PCT Appln. US01/28044
Alaiya et al., Cell Mol. Life Sci., 58:307-311, 2001.
An et al., Molec. Urol., 2:305-309, 1998.
Arap et al., Nature Med., 8:121-127. 2002.
Arap et al., Curr. Opin. Oncol., 10:560-565, 1998b.
Arap et al., Science, 279:377-380, 1998a.
Badalament et al., J. UroL., 156:1375-1380, 1996.
Baichwal and Sugden, In: Gene Transfer, Kucherlapati (Ed.), NY, Plenum Press, 117-148, 1986.
Bakhshi et al., Cell, 41:899-906, 1985.
Barany and Merrifield, In: The Peptides, Gross and Meienhofer (Eds.), Academic Press, NY, 1-284, 1979.
Barrow and Soothill, Trends Microbiol. 5:268-271, 1997.
Blanc et al., J. Immunol. Methods, 241:43-59, 2000.
Bova et al., Cancer Res., 53:3869-3873, 1993.
Brawn et al., The Prostate, 28: 295-299, 1996.

Brooks et al., Cell, 79:1157-1164, 1994.
Burg et al. Cancer Res., 59:2869-2874, 1999b.
Burg et al., Cancer Res., 58:2869-2874, 1999a.
Campbell et al, Gynecol. Oncol., 80:121-127, 2001b.
Campbell et al., Am. J. Pathol., 158:25-32, 2001.
Campbell et al., Am. J. Pathol., 158:25-32, 2001a.
Chen and Okayama, Mol. Cell. Biol., 7:2745-2752, 1987.
Cleary and Sklar, Proc. Natl. Acad. Sci. USA, 82:7439-43, 1985.
Coffin, In: Virology, Fields et al. (Eds.), Raven Press, NY, 1437-1500, 1990.
Coupar et al., Gene, 68:1-10, 1988.
Cumis et al., Nat. Biotechnol., 18:1185-1190, 2000.
De Rosa et al., Int. J Pharm., 242(1-2):225, 2002.
Douglas et al., Oncogene, 14:661-669, 1997.
Du and Williams, Blood, 89:3897-3908, 1997.
Eliceiri and Cheresh, Curr. Opin. Cell. Biol., 13:563-568, 2001.
Ellerby et al. Nature Med., 9:1032-1038, 1999.
Folkman, In: Cancer: Principles and Practice, eds. DeVita et al., 3075-3085, Lippincott-Raven, NY, 1997.
Folkman, Nature Biotechnol., 15: 510, 1997.
Folkman, Nature Med., 1:27-31, 1995.
Friedmann, Science, 244:1275-1281, 1989.
Gomez-Foix et al., J. Biol. Chem., 267:25129-25134, 1992.
Goodman & Gilman's “The Pharmacological Basis of Therapeutics” and
Gopal, Mol. Cell. Biol., 5:1188-1190, 1985.
Graham and Prevec, In: Methods in Molecular Biology: Gene Transfer and Expression Protocol, Murray (Ed.), Humana Press, NJ, 7:109-128, 1991.
Graham and van der Eb, Virology, 52:456-467, 1973.
Graham et al., J. Gen. Virol., 36:59-72, 1977.
Grunhaus and Horwitz, Seminar in Virology, 3:237-252, 1992.
Gupta et al., Proc. Am. Assn. Cancer Res., 38:554, 1997.
Hadigan et al., J. Amer. Med. Assn., 284:472-477, 2000.
Harlow and Lane, In: Antibodies: A Laboratory Manual, Cold Spring Harbor Laboratory Press, NY, 1988.
Hendrix, Current Biol., 9:914-917, 1999.
Hermonat and Muzycska, Proc. Natl. Acad. Sci. USA, 81:6466-6470, 1984.
Herz and Gerard, Proc. Natl. Acad. Sci. USA, 90:2812-2816, 1993.
Hong and Clayman, Cancer Res., 60:6551-6556, 2000.
Horwich, et al., J. Virol., 64:642-650, 1990.
Huang et al., Prostate, 23: 201-212, 1993.
Isaacs et al., Cancer Res., 51:4716-4720, 1991.
Isaacs et al., Sem. Oncol., 21:1-18, 1994.
Jain et al., Antiviral Res., 51:151-177, 2001
Johnson et al., In: Peptide Turn Mimetics, Pezzuto et al. (Eds.), Chapman and Hall, NY, 1993.
Jones and Shenk, Cell, 13:181-188, 1978.
Kang et al., Cancer Cell, 3:537-549, 2003.
Kerr et al., Br. J. Cancer, 26:239-257, 1972.
Koivunen et al. Methods Mol. Biol., 129:3-17, 1999b.
Koivunen et al., Nature Biotechnol., 17:768-774, 1999a
Kolonin et al., Curr. Opin. Chem. Biol. 5:308-13, 2001.
Larocca et al., FASEB J, 13:727-734, 1999.
Le Gal La Salle et al., Science, 259:988-990, 1993.
Levrero et al., Gene, 101:195-202, 1991.
Luo et al., Cancer Res., 62:2220-26, 2002.
Lutticken et al., Science, 263:89, 1994.
MacGregor and Caskey, Nucleic Acids Res., 17:2365, 1989.
Macoska et al., Cancer Res., 54:3824-3830, 1994.
Mann et al., Cell, 33:153-159, 1983.
Merrifield, Science, 232: 341-347, 1986
Murphy et al., Cancer, 78: 809-818, 1996.
Ni et al., J. Urol., 167:1859-62, 2002.
Nicolas and Rubinstein, In: Vectors. A survey of molecular cloning vectors and their uses, Rodriguez and Denhardt (Eds.), Stoneham: Butterworth, 494-513, 1988.
Nicolau et al., Methods Enzymol., 149:157-176, 1987.
O'Dowd et al., J. Urol., 158:687-698, 1997.
Orozco et al., Urology, 51:186-195, 1998.
Paglia et al., J. Interf Cytokine Res., 15:455-460, 1995.
Partin and Oesterling, J. Urol., 152:1358-1368, 1994.
Paskind et al., Virology, 67:242-248, 1975.
Pasqualini and Ruoslahti, Nature, 380:364-366, 1996.
Pasqualini et al. Nature Biotechnol., 15:542-546, 1997.
Pasqualini et al., Cancer Res., 60:722-727, 2000.
Pasqualini, J. Nucl. Med., 43:159-162, 1999.
Physicians Desk Reference
Piironen et al., Clin. Chem. 42:1034-1041, 1996.
Potter et al., Proc. Nat. Acad. Sci. USA, 81:7161-7165, 1984.
Poul and Marks, J. Mol. Biol., 288:203-211, 1999.
Racher et al., Biotechnology Techniques, 9:169-174, 1995.
Ragot et al., Nature, 361:647-650, 1993.
Rajotte and Ruoslahti, J. Biol. Chem., 274:11593-11598, 1999.
Rajotte et al., J Clin Invest 102:430-437, 1998.
Raulin et al., Prog. Lipid Res., 41:27-65, 2002.
Remington: The Science and Practice of Pharmacy,” 20th edition, Gennaro, Lippincott, 2000
Rich et al., Hum. Gene Ther., 4:461-476, 1993.
Ridgeway, In: Vectors: A Survey of Molecular Cloning Vectors and Their Uses, Rodriguez et al. (Eds.), Stoneham: Butterworth, 467-492, 1988.
Rippe et al., Mol. Cell. Biol., 10:689-695, 1990.
Rosenfeld et al., Cell, 68:143-155, 1992.
Rosenfeld et al., Science, 252:431-434, 1991.
Smith and Scott, Meth. Enzymol., 21:228-257, 1993.
Smith and Scott, Science, 228:1315-1317, 1985.
St. Croix, B. et al., Science, 289:1197-1202, 2000.
Stewart and Young, Solid Phase Peptide Synthesis, 2d. ed., Pierce Chemical Co., 1984.
Stratford-Perricaudet and Perricaudet, In: Human Gene Transfer, Cohen-Haguenauer et al. (Eds.) John Libbey Eurotext, France, 51-61, 1991.
Stratford-Perricaudet et al., Hum. Gene. Ther., 1:241-256, 1990.
Tam et al., J. Am. Chem. Soc., 105:6442, 1983.
Tamura et al., Science, 278:117-120, 1997.
Temin, In: Gene Transfer, Kucherlapati (Ed.), NY, Plenum Press, 149-188, 1986.
Thomas et al., Br. J. Urol., 77:367-372, 1996.
Triantafilou et al., Hum. Immunol., 62:764-770, 2001.
Tsujimoto et al., Nature, 315:340-343, 1985.
Tur-Kaspa et al., Mol. Cell. Biol., 6:716-718, 1986.
Veltri et al., Urology, 53:139-147, 1999.
Weitzman et al., Gene Ther. Vector Sys., 2:17-25, 1997.
Wickham, Gene Ther., 7:110-114, 2000.
Wong et al., Gene, 10: 87-94, 1980.
Wu and Wu, Biochemistry, 27:887-892, 1988.
Wu and Wu, J. Biol. Chem., 262:4429-4432, 1987.
Zhang, Cancer Gene Ther., 6:113-138, 1999.

1.-44. (canceled)

45. An isolated peptide that selectively binds glucose regulated protein 78 (GRP78), wherein the peptide comprises WIFPWIQL (SEQ ID NO:6) or WDLAWMFRLPVG (SEQ ID NO:7).

46. The isolated peptide of claim 45, wherein the isolated peptide is covalently coupled to a therapeutic agent.

47. The isolated peptide of claim 46, wherein the therapeutic agent is a drug, a chemotherapeutic agent, a radioisotope, a pro-apoptosis agent, an anti-angiogenic agent, a hormone, a cytokine, a cytotoxic agent, a cytocidal agent, a cytostatic agent, a peptide, a protein, an antibiotic, an antibody, a Fab fragment of an antibody, a hormone antagonist, a nucleic acid or an antigen.

48. The isolated peptide of claim 47, wherein the therapeutic agent is an anti-angiogenic agent selected from the group consisting of thrombospondin, angiostatin5, pigment epithelium-derived factor, angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, Docetaxel, polyamines, a proteasome inhibitor, a kinase inhibitor, a signaling peptide, accutin, cidofovir, vincristine, bleomycin, AGM-1470, platelet factor 4 and minocycline.

49. The isolated peptide of claim 47, wherein the therapeutic agent is a pro-apoptosis agent selected from the group consisting of etoposide, ceramide sphingomyelin, Bax, Bid, Bik, Bad, caspase-3, caspase-8, caspase-9, fas, fas ligand, fadd, fap-1, tradd, faf rip, reaper, apoptin, interleukin-2 converting enzyme, (KLAKLAK)₂ (SEQ ID NO:11) and annexin V.

50. The isolated peptide of claim 49, wherein the therapeutic agent is (KLAKLAK)₂ (SEQ ID NO:11).

51. The isolated peptide of claim 50, wherein the therapeutic agent is _{D}(KLAKLAK)₂.

52. The isolated peptide of claim 47, wherein the therapeutic agent is a cytokine selected from the group consisting of interleukin 1 (IL-1), IL-2, IL-5, IL-10, IL-12, IL-18, interferon-γ (IF-γ), IF α, IF-β, tumor necrosis factor-α (TNF-α), and GM-CSF (granulocyte macrophage colony stimulating factor).

53. The isolated peptide of claim 45, wherein the peptide is attached to a molecular complex.

54. The isolated peptide of claim 53, wherein the complex is a virus, a bacteriophage, a bacterium, a liposome, a microparticle, a magnetic bead, a yeast cell, a mammalian cell or a cell.

55. The isolated peptide of claim 54, wherein the complex is a virus or a bacteriophage.

56. The isolated peptide of claim 55, wherein the virus is selected from the group consisting of adenovirus, retrovirus and adeno-associated virus (AAV).

57. The isolated peptide of claim 55, wherein the virus is further defined as a gene therapy vector.

58. The isolated peptide of claim 45, wherein the peptide is comprised in a pharmaceutically acceptable composition.

59. A method of delivering a therapeutic or diagnostic agent to cells expressing GRP78 comprising obtaining a peptide in accordance with claim 45 linked to a therapeutic or diagnostic agent and contacting a cell population that comprises cells expressing GRP78 to thereby deliver the therapeutic or diagnostic agent to the cells.

60. The method of claim 59, wherein the cells are prostate cancer cells located in a subject and wherein the method is a therapeutic method for the treatment of prostate cancer and the peptide is administered in a therapeutically effective amount in a pharmaceutically acceptable carrier.

61. The method of claim 60, wherein the prostate cancer is metastatic prostate cancer.

62. The method of claim 60, wherein the subject is a mammal.

63. The method of claim 62, wherein the mammal is a human.

64. The method of claim 59, further defined as a diagnostic method for imaging cells expressing GRP78 comprising exposing cells to an isolated peptide that selectively binds GRP78, wherein the peptide is coupled to a diagnostic agent.

65. The method of claim 64, wherein the agent is a radioisotope or an imaging agent.

66. An isolated peptide that selectively binds GRP78, identified by a process comprising:
a) contacting a cell or tissue expressing GRP78 with a plurality of phage, wherein each phage comprises heterologous peptide sequences incorporated into a fiber protein,
b) removing the phage that do not bind to the cell or tissue expressing GRP78, and
c) isolating the phage that bind the cell or tissue expressing GRP78.

67. The peptide of claim 66, wherein the process is repeated at least twice.

68. The peptide of claim 67, wherein the process further comprises isolating and sequencing isolated phage nucleic acid.


SEQUENCE LISTING (14 sequences)

SEQ ID NO: 1 - protein, 9 aa, Artificial Sequence
  Description of Artificial Sequence  Synthetic peptide
CGRRAGGSC

SEQ ID NO: 2 - protein, 9 aa, Artificial Sequence
  Description of Artificial Sequence  Synthetic peptide
CRGSGAGRC

SEQ ID NO: 3 - protein, 9 aa, Artificial Sequence
  Description of Artificial Sequence  Synthetic peptide
CSGGGRARC

SEQ ID NO: 4 - protein, 9 aa, Artificial Sequence
  Description of Artificial Sequence  Synthetic peptide
CKGGRAKDC

SEQ ID NO: 5 - protein, 9 aa, Artificial Sequence
  Description of Artificial Sequence  Synthetic peptide
CGSPGWVRC

SEQ ID NO: 6 - protein, 8 aa, Artificial Sequence
  Description of Artificial Sequence  Synthetic peptide
WIFPWIQL

SEQ ID NO: 7 - protein, 12 aa, Artificial Sequence
  Description of Artificial Sequence  Synthetic peptide
WDLAWMFRLPVG

SEQ ID NO: 8 - protein, 8 aa, Artificial Sequence
  Description of Artificial Sequence  Synthetic peptide
CNVSDKSC

SEQ ID NO: 9 - protein, 5 aa, Artificial Sequence
  Description of Artificial Sequence  Synthetic peptide
CARAC

SEQ ID NO: 10 - protein, 9 aa, Artificial Sequence
  Description of Artificial Sequence  Synthetic peptide
CGDKAKGRC

SEQ ID NO: 11 - protein, 14 aa, Artificial Sequence
  Description of Artificial Sequence  Synthetic peptide
KLAKLAKKLAKLAK

SEQ ID NO: 12 - protein, 9 aa, Artificial Sequence
  Description of Artificial Sequence  Synthetic peptide
CVMGSVTGC

SEQ ID NO: 13 - DNA, 15 bp, Artificial Sequence
  Description of Artificial Sequence  Synthetic Primer
gtgagccggctgccc

SEQ ID NO: 14 - DNA, 15 bp, Artificial Sequence
  Description of Artificial Sequence  Synthetic Primer
ttcggccccagcggc